(19) United States
(12) Patent Application Publication
(10) Pub. No.: US 2006/0178513 A1
(43) Pub. Date: Aug. 10, 2006

(54) Pyridinylanilides

(76) Inventors: Ralf Dunkel, Monheim (DE); Hans-Ludwig Elbe, Wuppertal (DE); Benoit Hartmann, Langenfeld (DE); Joerg Nico Greul, Leichlingen (DE); Ulrike Wachendorff-Neumann, Neuwied (DE); Peter Dahmen, Neuss (DE); Karl-Heinz Kuck, Langenfeld (DE); Darren James Mansfield, Lyon (FR); Pierre-Yves Coqueron, Lyon (FR); Heiko Rieck, Lyon (FR); Philippe Desbordes, Lyon (FR)

Correspondence Address: BAYER CROPSCIENCE LP;Patent Department, 100 BAYER ROAD, PITTSBURGH, PA, 15205-9741, US

(21) Appl. No.: 10/563,725

(22) PCT Filed: Jul. 5, 2004
(86) PCT No.: PCT/EP04/07323

§ 371 Date: Apr. 18, 2006

(30) Foreign Application Priority Data
Jul. 10, 2003 (EP) 03015733.3

(51) Int. Cl. C07D 417/02; C07D 413/02; C07D 409/02; C07D 401/02

(52) U.S. Cl. 544/405; 546/271.4; 546/275.4; 546/280.1; 546/255; 546/283.4; 546/329; 546/269.7

(57) ABSTRACT

This invention relates to pyridinylanilides of formula (I)

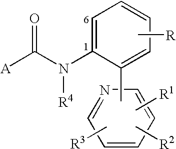

in which R, R¹, R², R³, R⁴ and A are as defined in the disclosure, to a plurality of processes for preparing these substances, to their use for controlling unwanted microorganisms, and to novel intermediates and their preparation.

The present invention relates to novel pyridinylanilides, to several processes for their preparation and to their use for controlling unwanted microorganisms.

It is already known that certain pyridinylanilides have fungicidal properties (cf. WO 01/53259 and JP-A 8-92223). Thus, for example, the pyridinylanilides N-[2-(2-chloro-3-pyridinyl)phenyl]-1,4-dimethyl-1H-pyrrole-3-carboxamide and 1,4-dimethyl-N-{2-[2-trifluoromethyl)-4-pyridinyl]phenyl}-1H-pyrrole-3-carboxamide (WO 01/53259) or 1-methyl-N-(2-pyridin-2-ylphenyl)-3-(trifluoromethyl)-1H-pyrazole-4-carboxamide and 2-chloro-N-2-pyridin-2-ylphenyl)nicotinamide (JP-A 8-92223) can be used for the control of fungi. The activity of such compounds, however, is not always satisfactory, particularly if they are applied at low dosages. Other pyridinylanilides, e.g. N-[2-(6-bromo-2-pyridinyl)-4-methylphenyl]-2,2-dimethylpropanamide, N-{4-methyl-2-[6-trifluoromethyl)-2-pyridinyl]phenyl}cyclopropanecarboxamide and N-{4methyl-2-[6-trifluoromethyl)-2-pyridinyl]phenyl}benzamide, are known as herbicides and plant growth regulators (cf. WO 95/09846).

This invention is directed to novel pyridinylanilides of the formula (I) 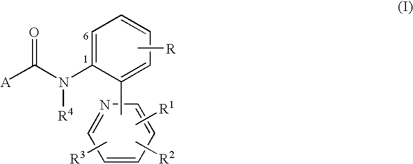
in which
R represents hydrogen, fluorine, chlorine, methyl or trifluoromethyl;
R¹, R² and R³ independently of one another each represents hydrogen, halogen, cyano, nitro, amino, hydroxyl, formyl, carboxyl, carbamoyl, thiocarbamoyl;
   or represents in each case straight-chain or branched alkyl, hydroxyalkyl, oxoalkyl, alkoxy, alkoxyalkyl, alkylthioalkyl, dialkoxyalkyl, alkylthio, alkylsulfinyl or alkylsulfonyl having in each case 1 to 8 carbon atoms in the respective alkyl moiety;
   or represents in each case straight-chain or branched alkenyl or alkenyloxy having in each case 2 to 6 carbon atoms;
   or represents in each case straight-chain or branched halogenoalkyl, halogenoalkoxy, halogenoalkylthio, halogenoalkylsulfinyl or halogenoalkylsulfonyl having in each case 1 to 6 carbon atoms and 1 to 13 identical or different halogen atoms;
   or represents in each case straight-chain or branched halogenoalkenyl or halogenoalkenyloxy having in each case 2 to 6 carbon atoms and 1 to 11 identical or different halogen atoms;
or represents in each case straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, dialkylaminocarbonyloxy having 1 to 6 carbon atoms in the respective hydrocarbon chain, alkenylcarbonyl or alkynylcarbonyl having 2 to 6 carbon atoms in the respective hydrocarbon chain;
   or represents cycloalkyl or cycloalkyloxy having in each case 3 to 6 carbon atoms;
   or represents the grouping —C(Q¹)=N-Q², wherein
      Q¹ represents hydrogen, hydroxyl or C₁-C₄-alkyl, C₁-C₄-halogenoalkyl having 1 to 9 identical or different halogen atoms, or C₃-C₆-cycloalkyl and
      Q² represents hydroxyl, amino, methylamino, phenyl, benzyl; or represents C₁-C₄-alkyl or C₁-C₄-alkoxy, each of which is optionally substituted by halogen, cyano, hydroxyl, C₁-C₄-alkoxy, C₁-C₄-alkylthio, C₁-C₄-alkylamino, di(C₁-C₄-alkyl)amino or phenyl; or represents C₂-C₄-alkenyloxy or C₂-C₄-alkynyloxy,
   represents phenyl, phenoxy, phenylthio, benzoyl, benzoylethenyl, cinnamoyl, heterocyclyl or phenylalkyl, phenylalkyloxy, phenylalkylthio or heterocyclylalkyl having in each case 1 to 3 carbon atoms in the respective alkyl moieties, each of which is optionally mono- to tri-substituted, identically or differently, in the ring moiety by halogen, in each case straight-chain or branched C₁-C₄-alkyl and C₁-C₄-alkoxy;
      or
R² and R³, if attached to the pyridinyl moiety in ortho position to each other, furthermore together represent C₃-C₄-alkylene, C₃-C₄-alkenylene, C₂-C₃-oxyalkylene or C₁-C₂-dioxyalkylene, in each case optionally mono- to tetra-substituted, identically or differently, by fluorine, chlorine, oxo, methyl, ethyl, trifluoromethyl;
R⁴ represents hydrogen, C₁-C₈-alkyl, C₁-C₆-alkylsulfinyl, C₁-C₆-alkylsulfonyl, C₁-C₄-alkoxy-C₁-C₄-alkyl, C₃-C₈-cycloalkyl; C₁-C₆-halogenoalkyl, C₁-C₄-halogenoalkylthio, C₁-C₄-halogenoalkylsulfinyl, C₁-C₄-halogenoalkylsulfonyl, halogeno-C₁-C₄-alkoxy-C₁-C₄-alkyl, C₃-C₈-halogenocycloalkyl having in each case 1 to 9 fluorine-, chlorine- and/or bromine atoms; formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C₃-halogenoalkyl)carbonyl-C₁-C₃-alkyl, (C₁-C₃-halogenoalkoxy)carbonyl-C₁-C₃-alkyl having in each case 1 to 7 fluorine-, chlorine- and/or bromine atoms, (C₁-C₃-alkyl)carbonyl-C₁-C₃-halogenoalkyl, (C₁-C₃-alkoxy)carbonyl-C₁-C₃-halogenoalkyl having in each case 1 to 6 fluorine-, chlorine- and/or bromine atoms, (C₁-C₃-halogenoalkyl)carbonyl-C₁-C₃-halogenoalkyl, (C₁-C₃-halogenoalkoxy)carbonyl-C₁-C₃-halogenoalkyl having in each case 1 to 13 fluorine-, chlorine- and/or bromine atoms; —COR⁵, —CONR⁶R⁷ or —CH₂NR⁸R⁹,
R⁵ represents hydrogen, C₁-C₈-alkyl, C₁-C₈-alkoxy, C₁-C₄-alkoxy-C₁-C₄-alkyl, C₃-C₈-cycloalkyl; C₁-C₆-halogenoalkyl, C₁-C₆-halogenoalkoxy, halogeno-C₁-C₄-alkoxy-C₁-C₄-alkyl, C₃-C₈-halogenocycloalkyl having in each case 1 to 9 fluorine-, chlorine- and/or bromine atoms; or —COR¹⁰,
R⁶ and R⁷ independently of one another each represent hydrogen, C₁-C₈-alkyl, C₁-C₄-alkoxy-C₁-C₄-alkyl, C₃-C₈-cycloalkyl; C₁-C₈-halogenoalkyl, halogeno-C₁-C₄-alkoxy-C₁-C₄-alkyl, C₃-C₈-halogenocycloalkyl having in each case 1 bis 9 fluorine-, chlorine- and/or bromine atoms,
R⁶ and R⁷ furthermore together with the nitrogen atom to which they are attached, represent a saturated 5- to 8-membered heterocycle, which heterocycle may have 1 or 2 additional, non-adjacent heteroatoms selected from the group consisting of oxygen, sulphur and NR¹¹, and which heterocycle may optionally be mono- to poly-substituted, identically or differently, by halogen or C₁-C₄-alkyl,
R⁸ and R⁹ independently of one another each represent hydrogen, C₁-C₈-alkyl, C₃-C₈-cycloalkyl; C₁-C₈-halogenoalkyl, C₃-C₈-halogenocycloalkyl having in each case 1 to 9 fluorine-, chlorine- and/or bromine atoms,
R⁸ and R⁹ furthermore together with the nitrogen atom to which they are attached, represent a saturated 5- to 8-membered heterocycle, which heterocycle may have 1 or 2 additional, non-adjacent heteroatoms selected from the group consisting of oxygen, sulphur and NR¹¹, and which heterocycle may optionally be mono- to poly-substituted, identically or differently, by halogen or C₁-C₄-alkyl,
R¹⁰ represents hydrogen, C₁-C₈-alkyl, C₁-C₈-alkoxy, C₁-C₄-alkoxy-C₁-C₄-alkyl, C₃-C₈-cycloalkyl; C₁-C₆-halogenoalkyl, C₁-C₆-halogenoalkoxy, halogeno-C₁-C₄-alkoxy-C₁-C₄-alkyl, C₃-C₈-halogenocycloalkyl having in each case 1 to 9 fluorine-, chlorine- and/or bromine atoms,
R¹¹ represents hydrogen or C₁-C₆-alkyl,

A represents a radical of the formula (A1) 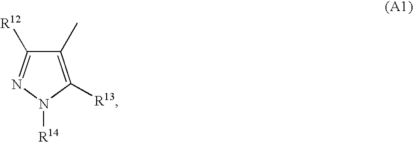
wherein
R¹² represents hydrogen, cyano, halogen, nitro, C₁-C₄-alkyl, C₁-C₄-alkoxy, C₁-C₄-alkyl-thio, C₃-C₆-cycloalkyl, C₁-C₄-halogenoalkyl, C₁-C₄-halogenoalkoxy or C₁-C₄-halogenoalkylthio each having 1 to 5 halogen atoms, aminocarbonyl or aminocarbonyl-C₁-C₄-alkyl and
R¹³ represents hydrogen, halogen, cyano, C₁-C₄-alkyl, C₁-C₄-alkoxy or C₁-C₄-alkylthio and
R¹⁴ represents hydrogen, C₁-C₄-alkyl, hydroxy-C₁-C₄-alkyl, C₂-C₆-alkenyl, C₃-C₆-cyclo-alkyl, C₁-C₄-alkylthio-C₁-C₄-alkyl, C₁-C₄-alkoxy-C₁-C₄-alkyl, C₁-C₄-halogenoalkyl, C₁-C₄-halogenoalkylthio-C₁-C₄-alkyl, C₁-C₄-halogenoalkoxy-C₁-C₄-alkyl each having 1 to 5 halogen atoms, or phenyl,
   or
A represents a radical of the formula (A2) 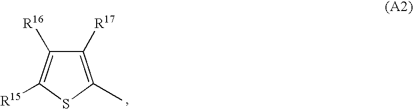
   wherein
R¹⁵ and R¹⁶ independently of one another each represent hydrogen, halogen, C₁-C₄-alkyl or C₁-C₄-halogenoalkyl having 1 to 5 halogen atoms and
R¹⁷ represents halogen, cyano or C₁-C₄-alkyl, or C₁-C₄-halogenoalkyl or C₁-C₄-halogeno-alkoxy each having 1 to 5 halogen atoms,
   or
A represents a radical of the formula (A3) 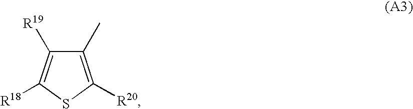
   wherein
R¹⁸ and R¹⁹ independently of one another each represent hydrogen, halogen, C₁-C₄-alkyl or C₁-C₄-halogenoalkyl having 1 to 5 halogen atoms and
R²⁰ represents hydrogen, halogen, C₁-C₄-alkyl or C₁-C₄-halogenoalkyl having 1 to 5 halogen atoms,
   or
A represents a radical of the formula (A4) 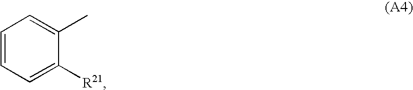
   wherein
R²¹ represents hydrogen, halogen, hydroxyl, cyano, C₁-C₆-alkyl, C₁-C₄-halogenoalkyl, C₁-C₄-halogenoalkoxy or C₁-C₄-halogenoalkylthio each having 1 to 5 halogen atoms,
   or
A represents a radical of the formula (A5) 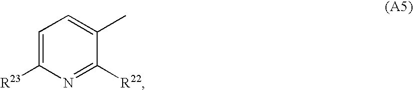
   wherein
R²² represents halogen, hydroxyl, cyano, C₁-C₄-alkyl, C₁-C₄-alkoxy, C₁-C₄-alkylthio, C₁-C₄-halogenoalkyl, C₁-C₄-halogenoalkylthio or C₁-C₄-halogenoalkoxy each having 1 to 5 halogen atoms and
R²³ represents hydrogen, halogen, cyano, C₁-C₄-alkyl, C₁-C₄-alkoxy, C₁-C₄-alkylthio, C₁-C₄-halogenoalkyl, C₁-C₄-halogenoalkoxy each having 1 to 5 halogen atoms, C₁-C₄-alkylsulphinyl or C₁-C₄-alkylsulphonyl,
   or
A represents a radical of the formula (A6) 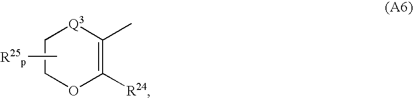
   wherein
R²⁴ represents C₁-C₄-alkyl or C₁-C₄-halogenoalkyl having 1 to 5 halogen atoms and
R²⁵ represents C₁-C₄-alkyl,
Q³ represents a sulphur or oxygen atom, represents SO, SO₂ or CH₂,
p represents 0, 1 or 2, where R²⁵ represents identical or different radicals if p represents 2,
   or
A represents a radical of the formula (A7) 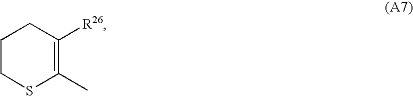
   wherein
R²⁶ represents C₁-C₄-alkyl or C₁-C₄-halogenoalkyl having 1 to 5 halogen atoms,
   or
A represents a radical of the formula (A8) 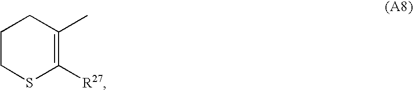
   wherein
R²⁷ represents C₁-C₄-alkyl or C₁-C₄-halogenoalkyl having 1 to 5 halogen atoms,
   or
A represents a radical of the formula (A9) 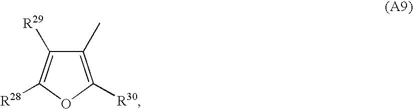
   wherein
R²⁸ and R²⁹ independently of one another each represent hydrogen, halogen, amino, C₁-C₄-alkyl or C₁-C₄-halogenoalkyl having 1 to 5 halogen atoms and
R³⁰ represents hydrogen, halogen, C₁-C₄-alkyl or C₁-C₄-halogenoalkyl having 1 to 5 halogen atoms,
   or
A represents a radical of the formula (A10) 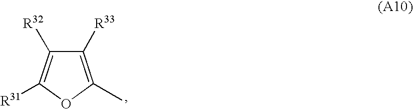
   wherein
R³¹ and R³² independently of one another each represent hydrogen, halogen, amino, nitro, C₁-C₄-alkyl or C₁-C₄-halogenoalkyl having 1 to 5 halogen atoms and
R³³ represents hydrogen, halogen, C₁-C₄-alkyl or C₁-C₄-halogenoalkyl having 1 to 5 halogen atoms,
   or
A represents a radical of the formula (A11) 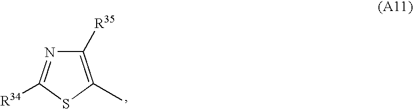
   wherein
R³⁴ represents hydrogen, halogen, amino, C₁-C₄-alkylamino, di-(C₁-C₄-alkyl)amino, cyano, C₁-C₄-alkyl or C₁-C₄-halogenoalkyl having 1 to 5 halogen atoms and
R³⁵ represents halogen, C₁-C₄-alkyl or C₁-C₄-halogenoalkyl having 1 to 5 halogen atoms,
   or
A represents a radical of the formula (A12) 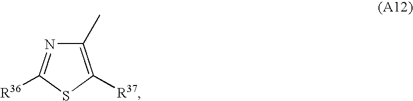
   wherein
R³⁶ represents hydrogen, halogen, amino, C₁-C₄-alkylamino, di-(C₁-C₄-alkyl)amino, cyano, C₁-C₄-alkyl or C₁-C₄-halogenoalkyl having 1 to 5 halogen atoms and
R³⁷ represents halogen, C₁-C₄-alkyl or C₁-C₄-halogenoalkyl having 1 to 5 halogen atoms,
   or
A represents a radical of the formula (A13) 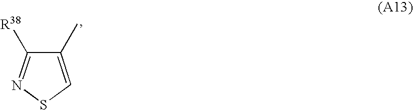
   wherein
R³⁸ represents halogen, C₁-C₄-alkyl or C₁-C₄-halogenoalkyl having 1 to 5 halogen atoms,
   or
A represents a radical of the formula (A14) 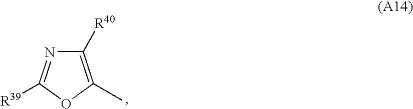
   wherein
R³⁹ represents hydrogen or C₁-C₄-alkyl and
R⁴⁰ represents halogen or C₁-C₄-alkyl,
   or
A represents a radical of the formula (A15) 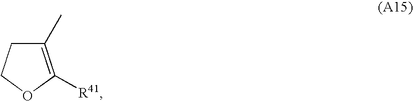
   wherein
R⁴¹ represents C₁-C₄-alkyl or C₁-C₄-halogenoalkyl having 1 to 5 halogen atoms,
   or
A represents a radical of the formula (A16) 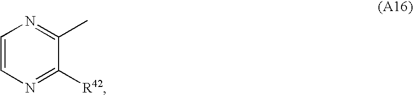
   wherein
R⁴² represents hydrogen, halogen, C₁-C₄-alkyl or C₁-C₄-halogenoalkyl having 1 to 5 halogen atoms,
   or
A represents a radical of the formula (A17) 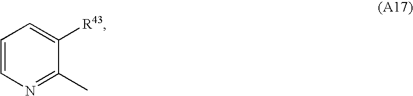
   wherein
R⁴³ represents halogen, hydroxyl, C₁-C₄-alkyl, C₁-C₄-alkoxy, C₁-C₄-alkylthio, C₁-C₄-halogenoalkyl, C₁-C₄-halogenoalkylthio or C₁-C₄-halogenoalkoxy each having 1 to 5 halogen atoms,
   excluded compounds of the formula (I), in which
R represents hydrogen and
R¹, R² and R³ independently of one another each represents hydrogen, halogen; or straight-chain or branched alkyl having 1 to 4 carbon atoms; or straight-chain or branched halogenoalkyl having 1 to 4 carbon atoms; and
R⁴ represents hydrogen
   and
A represents a radical of the formula (A1) 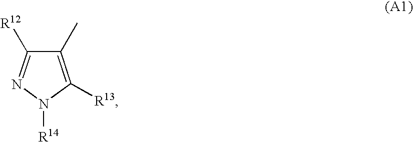
   wherein
R¹² represents halogen, C₁-C₄-alkyl, C₁-C₄-halogenoalkyl and
R¹³ represents hydrogen and
R¹⁴ represents methyl,
   or
A represents a radical of the formula (A2) 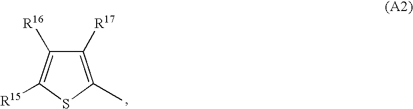
   wherein
R¹⁵ and R¹⁶ independently of one another each represent hydrogen or C₁-C₄-alkyl and
R¹⁷ represents halogen, C₁-C₄-alkyl or C₁-C₄-halogenoalkyl,
   or
A represents a radical of the formula (A4) 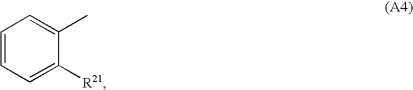
   wherein
R²¹ represents halogen, C₁-C₄-alkyl or C₁-C₄-halogenoalkyl,
   or
A represents a radical of the formula (A5) 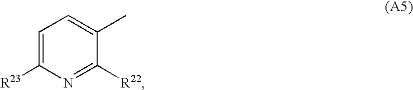
   wherein
R²² represents halogen and
R²³ represents hydrogen,
   or
A represents a radical of the formula (A6) 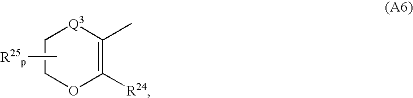
   wherein
R²⁴ represents methyl and
Q³ represents a sulphur or CH₂,
p represents 0,
   or
A represents a radical of the formula (A9) 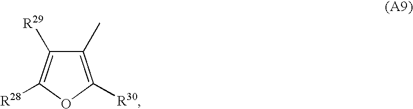
   wherein
R²⁸ and R²⁹ independently of one another each represent hydrogen or C₁-C₄-alkyl and
R³⁰ represents methyl,
   or
A represents a radical of the formula (A11) 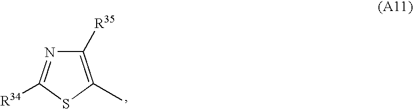
   wherein
R³⁴ represents hydrogen or C₁-C₄-alkyl and
R³⁵ represents halogen, C₁-C₄-alkyl or C₁-C₄-halogenoalkyl,
   or
A represents a radical of the formula (A16) 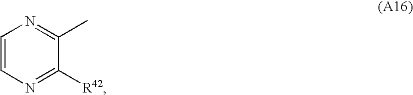
   wherein
R⁴² represents halogen.

The excluded compounds are known from JP-A 8-92223.

In each case the following single compounds known from JP-A 8-92223 are explicitly excluded from the scope of protection:
2-methyl-N-(2-pyridin-2-ylphenyl)-4-(trifluoromethyl)-1,3-thiazole-5-carboxamide;
N-(2-pyridin-2-ylphenyl)-4-(trifluoromethyl)-1,3-thiazole-5-carboxamide;
4-iodo-N-(2-pyridin-2-ylphenyl)-1,3-thiazole-5-carboxamide;
1-methyl-N-(2-pyridin-2-ylphenyl)-3-(trifluoromethyl)-1H-pyrazole-4-carboxamide;
3-iodo-1-methyl-N-(2-pyridin-2-ylphenyl)-1H-pyrazole-4-carboxamide;
2-methyl-N-(2-pyridin-2-ylphenyl)-3-furamide;
3-methyl-N-(2-pyridin-2-ylphenyl)thiophene-2-carboxamide;
2-chloro-N-(2-pyridin-2-ylphenyl)benzamide;
2-chloro-N-2-pyridin-2-ylphenyl)nicotinamide;
3-chloro-N-(2-pyridin-2-ylphenyl)pyrazine-2-carboxamide;
2-methyl-N-(2-pyridin-2-ylphenyl)-5,6-dihydro-1,4-oxathiine-3-carboxamide;
2-chloro-N-(2-pyridin-3-ylphenyl)nicotinamide;
2-chloro-N-(2-pyridin-4-ylphenyl)nicotinamide;
2-methyl-N-[2-(6-methylpyridin-2-yl)phenyl]-4-(trifluoromethyl)-1,3-thiazole-5-carboxamide;
1-methyl-N-[2-(6-methylpyridin-2-yl)phenyl]-3-(trifluormethyl)-1H-pyrazole-4H-carboxamide.

The compounds according to the invention may exist in different isomeric forms, in particular in form of stereoisomers, such as for example E- and Z-, threo- and erythro-, optical isomers and optionally in form of tautomers. The invention relates to all the use of the pure isomers as well as the E- and Z-isomers, the threo- and erythro-isomers, the optical isomers, optional mixtures of these isomers and the possible tautomeric forms.

Furthermore, it has been found that pyridinylanilides of the formula (I) are obtained when

a) carboxylic acid derivatives of the formula (II) 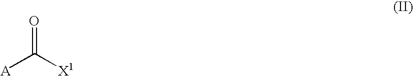
in which
X¹ represents halogen or hydroxyl and
A is as defined above,
are reacted with amines of the formula (III) 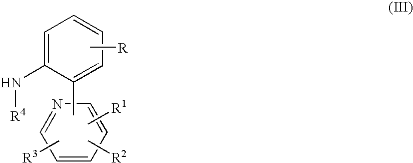
in which R, R¹, R², R³ and R⁴ are as defined above,
if appropriate in the presence of a catalyst, if appropriate in the presence of a condensing agent, if appropriate in the presence of an acid binder and if appropriate in the presence of a diluent,
   or

b) halogeno-carboxamides of the formula (IV) 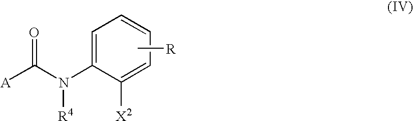
in which
R, R⁴ and A are as defined above, and
X² represents bromine or iodine,
are reacted with boronic acid derivatives of the formula (V) 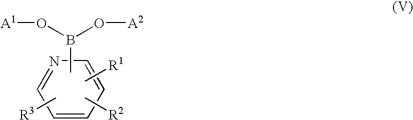
in which
R¹, R² and R³ are as defined above, and
A¹ and A² each represent hydrogen or together represent tetramethylethylene,
in the presence of a catalyst, if appropriate in the presence of an acid binder and if appropriate in the presence of a diluent,
   or

c) carboxamide boronic acid derivatives of the formula (VI) 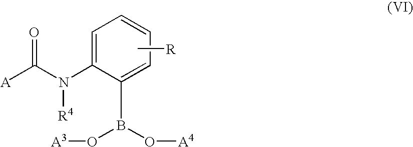
in which
R, R⁴ and A are as defined above, and
A³ and A⁴ each represent hydrogen or together represent tetramethylethylene,
are reacted with pyridinyl derivatives of the formula (VII) 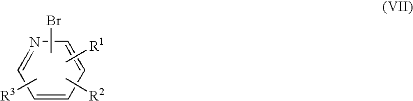
in which R¹, R² and R³ are as defined above,
in the presence of a catalyst, if appropriate in the presence of an acid binder and if appropriate in the presence of a diluent,
   or

d) halogeno-carboxamides of the formula (IV) 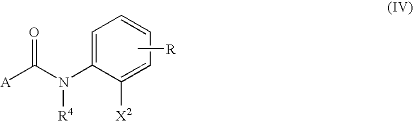
in which
R, R⁴ and A are as defined above, and
X² represents bromine or iodine,
are reacted with pyridinyl derivatives of the formula (VII) 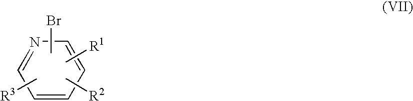
in which R¹, R² and R³ are as defined above,
in the presence of a palladium or platinum catalyst and in the presence of 4,4,4′,4′,5,5,5′,5′-octamethyl-2,2′-bis-1,3,2-dioxaborolane [bis(pinacolato)diboron], if appropriate in the presence of an acid binder and if appropriate in the presence of a diluent,
   or

e) pyridinylanilides of the formula (I-1) 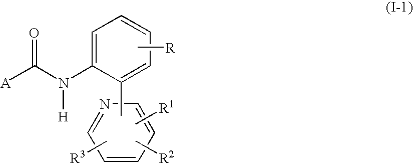
in which R, R¹, R², R³ and A are as defined above,
are reacted with halogenides of the formula (VIII)
   R^{4a—X}³ (VIII)
in which
X³ represents chlorine, bromine or iodine,
R^{4a} represents C₁-C₈-alkyl, C₁-C₆-alkylsulfinyl, C₁C₆-alkylsulfonyl, C₁-C₄-alkoxy-C₁C₄-alkyl, C₃-C₈-cycloalkyl; C₁-C₆-halogenoalkyl, C₁-C₄-halogenoalkylthio, C₁-C₄-halogenoalkylsulfinyl, C₁-C₄-halogenoalkylsulfonyl, halogeno-C₁-C₄-alkoxy-C₁-C₄-alkyl, C₃-C₈-halogenocycloalkyl having in each case 1 to 9 fluorine-, chlorine- and/or bromine atoms; formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C₃-halogenoalkyl)carbonyl-C₁-C₃-alkyl, (C₁-C₃-halogenoalkoxy)carbonyl-C₁-C₃-alkyl having in each case 1 to 7 fluorine-, chlorine- and/or bromine atoms, (C₁-C₃-alkyl)carbonyl-C₁-C₃-halogenoalkyl, (C₁-C₃-alkoxy)-carbonyl-C₁-C₃-halogenoalkyl having in each case 1 to 6 fluorine-, chlorine- and/or bromine atoms, (C₁-C₃-halogenoalkyl)carbonyl-C₁-C₃-halogenoalkyl, (C₁-C₃-halogenoalkoxy)carbonyl-C₁-C₃-halogenoalkyl having in each case 1 to 13 fluorine-, chlorine- and/or bromine atoms; —COR⁵, —CONR⁶R⁷ or —CH₂NR⁸R⁹,
R⁵, R⁶, R⁷, R⁸ and R⁹ are as defined above,
in the presence of a base and in the presence of a diluent.

Finally, it has been found that the novel pyridinylanilides of the formula (I) have very good microbicidal properties and can be used for controlling unwanted microorganisms both in crop protection and in the protection of materials.

Surprisingly, the pyridinylanilides of the formula (I) according to the invention have considerably better fungicidal activity than the constitutionally most similar active compounds of the prior art having the same direction of action.

The formula (I) provides a general definition of the pyridinylanilides according to the invention. Preferred definitions of the radicals of the above and/or below mentioned formulae are given in the following. These definitions apply in the same way to the final products of the formula (I) as well as to all intermediates.
R preferably represents hydrogen.
R furthermore preferably represents fluorine, which fluorine particularly preferably is placed in 4-, 5- or 6-position, very particularly preferably in 4- or 6-position of the anilide moiety [cf. formula (I) above].
R furthermore preferably represents chlorine, which chlorine particularly preferably is placed in 5-position of the anilide moiety [cf. formula (I) above].
R furthermore preferably represents methyl, which methyl particularly preferably is placed in 3-position of the anilide moiety [cf. formula (I) above].
R furthermore preferably represents trifluoromethyl, which trifluoromethyl particularly preferably is placed in 4- or 5-position of the anilide moiety [cf. formula (I) above].
R¹, R² and R³ independently of one another each preferably represents hydrogen, halogen, cyano, nitro, amino, hydroxyl, formyl, carboxyl, carbamoyl, thiocarbamoyl;
   or preferably represents in each case straight-chain or branched alkyl, alkoxy, alkoxyalkyl, alkylthioalkyl, alkylthio, alkylsulfinyl or alkylsulfonyl having in each case 1 to 6 carbon atoms;
   or preferably represents in each case straight-chain or branched halogenoalkyl, halogenoalkoxy, halogenoalkylthio, halogenoalkylsulfinyl or halogenoalkylsulfonyl having in each case 1 to 4 carbon atoms and 1 to 9 identical or different halogen atoms;
   or preferably represents in each case straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, dialkylaminocarbonyloxy having 1 to 4 carbon atoms in the respective hydrocarbon chain;
   or preferably represents cycloalkyl or cycloalkyloxy having in each case 3 to 6 carbon atoms;
   or preferably represents the grouping —C(Q¹)=N-Q², wherein
      Q¹ preferably represents hydrogen, hydroxyl or C₁-C₄-alkyl, C₁-C₄-halogenoalkyl having 1 to 9 identical or different halogen atoms or C₃-C₆-cycloalkyl and
      Q² preferably represents hydroxyl, C₁-C₄-alkyl, C₁-C₄-alkoxy, C₁-C₄-halogenoalkyl or C₁-C₄-halogenoalkoxy each having 1 to 9 identical or different halogen atoms.
R² and R³, if attached to the pyridinyl moiety in ortho position to each other, furthermore together preferably represent —(CH₂)₃—, —(CH₂)₄—, —CH═CH—CH═CH—, —O(CH₂)₂—, —O(CH₂)₃—, —OCH₂O—, —O(CH₂)₂O—, in each case optionally mono- to tetra-substituted, identically or differently, by fluorine, chlorine, oxo, methyl, ethyl, trifluoromethyl.
R¹, R² and R³ independently of one another each particularly preferably represents hydrogen, fluorine, chlorine, bromine, cyano; methyl, ethyl, n- or iso-propyl, n-, iso-, sec- or tert-butyl, methoxy, ethoxy, n- or iso-propoxy, n-, iso-, sec- or tert-butoxy, methylthio, ethylthio, n- or iso-propylthio, n-, iso-, sec- or tert-butylthio, trifluoromethyl, trifluoroethyl, difluoromethoxy, trifluoromethoxy, difluorochloromethoxy, trifluoroethoxy, cyclopropyl, cyclopentyl, cyclohexyl, or particularly preferably represents the grouping —C(Q¹)=N-Q², wherein
   Q¹ particularly preferably represents hydrogen, methyl, ethyl, trifluoromethyl or cyclopropyl, and
   Q² particularly preferably represents hydroxyl, methoxy, ethoxy, n-propoxy or iso-propoxy.
R² and R³, if attached to the pyridinyl moiety in ortho position to each other, furthermore together particularly preferably represent —CH₂)₃—, —(CH₂)₄—, —CH═CH—CH═CH—, —OCH₂O—, —O(CH₂)₂O—, —OCF₂O—, —O(CF₂)₂O—.
R¹, R² and R³ independently of one another each very particularly preferably represents hydrogen, fluorine, chlorine, bromine, iodine, methyl, ethyl, n- or iso-propyl, n-, iso-, sec- or tert-butyl, methoxy, ethoxy, n- or iso-propoxy, trifluoromethyl, trifluoroethyl, difluoromethoxy, trifluoromethoxy, difluorochloromethoxy, trifluoroethoxy,
   or very particularly preferably represents the grouping —C(Q¹)=N-Q², wherein
      Q¹ very particularly preferably represents hydrogen, methyl or ethyl and
      Q² very particularly preferably represents hydroxyl, methoxy, ethoxy, n-propoxy or iso-propoxy.
R² and R³, if attached to the pyridinyl moiety in ortho position to each other, furthermore together very particularly preferably represent —CH═CH—CH═CH—, —OCF₂—, —O(CF₂)₂O—.
R⁴ preferably represents hydrogen; C₁-C₆-alkyl, C₁-C₄-alkylsulfinyl, C₁-C₄-alkylsulfonyl, C₁-C₃-alkoxy-C₁-C₃-alkyl, C₃-C₆-cycloalkyl; C₁-C₄-halogenoalkyl, C₁-C₄-halogenoalkylthio, C₁-C₄-halogenoalkylsulfinyl, C₁-C₄-halogenoalkylsulfonyl, halogeno-C₁-C₃-alkoxyl-C₁-C₃-alkyl, C₃-C₆-halogenocycloalkyl having in each case 1 to 9 fluorine-, chlorine- and/or bromine atoms; formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C₃-halogenoalkyl)carbonyl-C₁-C₃-alkyl, (C₁-C₃-halogenoalkoxy)carbonyl-C₁-C₃-alkyl having in each case 1 to 7 fluorine-, chlorine- and/or bromine atoms, (C₁-C₃-alkyl)carbonyl-C₁-C₃-halogenoalkyl, (C₁-C₃-alkoxy)carbonyl-C₁-C₃-halogenoalkyl having in each case 1 to 6 fluorine-, chlorine- and/or bromine atoms, (C₁-C₃-halogenoalkyl)carbonyl-C₁-C₃-halogenoalkyl, (C₁-C₃-halogenoalkoxy)carbonyl-C₁-C₃-halogenoalkyl having in each case 1 to 13 fluorine-, chlorine- and/or bromine atoms; —COR⁵, —CONR⁶R⁷ or —CH₂NR⁸R⁹.
R⁴ particularly preferably represents hydrogen, methyl, ethyl, n- or iso-propyl, n-, iso-, sec- or tert-butyl, pentyl or hexyl, methylsulfinyl, ethylsulfinyl, n- or iso-propylsulfinyl, n-, iso-, sec- or tert-butylsulfinyl, methylsulfonyl, ethylsulfonyl, n- or iso-propylsulfonyl, n-, iso-, sec- or tert-butylsulfonyl, methoxymethyl, methoxyethyl, ethoxymethyl, ethoxyethyl, cyclopropyl, cyclopentyl, cyclohexyl, trifluoromethyl, trichloromethyl, trifluoroethyl, difluoromethylthio, difluorochloromethylthio, trifluoromethylthio, trifluoromethylsulfinyl, trifluoromethylsulfonyl, trifluoromethoxymethyl; —CH₂—CHO, —CH₂CH₂—CHO, —CH₂—CO—CH₃, —CH₂-CO—CH₂CH₃, —CH₂—CO—CH(CH₃)₂, —CH₂CH₂—CO—CH₃, —CH₂CH₂—CO—CH₂CH₃, —CH₂CH₂—CO—CH(CH₃)₂, —CH₂—C(O)OCH₃, —CH₂—C(O)OCH₂CH₃, —CH₂—C(O)OCH(CH₃)₂, —CH₂CH₂—C(O)OCH₃, —CH₂CH₂—C(O)OCH₂CH₃, —CH₂CH₂—C(O)OCH(CH₃)₂, —CH₂—CO—CF₃, —CH₂—CO—CCl₃, —CH₂—CO—CH₂CF₃, —CH₂—CO—CH₂CCl₃, —CH₂CH₂—CO—CH₂CF₃, —CH₂CH₂—CO—CH₂CCl₃, —CH₂—C(O)OCH₂CF₃, —CH₂—C(O)OCF₂CF₃, —CH₂—C(O)OCH₂CCl₃, —CH₂—C(O)OCCl₂CCl₃, —CH₂CH₂—C(O)OCH₂CF₃, —CH₂CH₂—C(O)OCF₂CF₃, —CH₂CH₂—C(O)OCH₂CCl₃, —CH₂CH₂—C(O)O—CCl₂CCl₃; —COR⁵, —CONR⁶R⁷ or —CH₂NR⁸R⁹.
R⁴ very particularly preferably represents hydrogen; methyl, methoxymethyl, —CH₂—CHO, —CH₂CH₂—CHO, —CH₂—CO—CH₃, —CH₂—CO—CH₂CH₃, —CH₂—CO—CH(CH₃)₂ or —COR⁵.
R⁵ preferably represents hydrogen, C₁-C₆-alkyl, C₁-C₄-alkoxy, C₁-C₃-alkoxy-C₁-C₃-alkyl, C₃-C₆-cycloalkyl; C₁-C₄-halogenoalkyl, C₁-C₄-halogenoalkoxy, halogeno-C₁-C₃-alkoxy-C₁-C₃-alkyl, C₃-C₆-halogenocycloalkyl having in each case 1 to 9 fluorine-, chlorine- and/or bromine atoms; or —COR¹⁰.
R⁵ particularly preferably represents hydrogen, methyl, ethyl, n- or iso-propyl, tert-butyl, methoxy, ethoxy, tert-butoxy, cyclopropyl; trifluoromethyl, trifluoromethoxy; or —COR¹⁰.
R⁵ very particularly preferably represents hydrogen, —COCH₃, —CHO, —COCH₂OCH₃, —COCO₂CH₃, —COCO₂CH₂CH₃; or —COR¹⁰.
R⁶ and R⁷ independently of one another each preferably represent hydrogen, C₁-C₆-alkyl, C₁-C₃-alkoxy-C₁-C₃-alkyl, C₃-C₆-cycloalkyl; C₁-C₄-halogenoalkyl, halogeno-C₁-C₃-alkoxy-C₁-C₃-C₁-C₃-alkyl, C₃-C₆-halogenocycloalkyl having in each case 1 to 9 fluorine-, chlorine- and/or bromine atoms.
R⁶ and R⁷ furthermore together with the nitrogen atom to which they are attached, preferably represent a saturated 5- to 8-membered heterocycle, which heterocycle may have 1 or 2 additional, non-adjacent heteroatoms selected from the group consisting of oxygen, sulphur and NR¹¹, and which heterocycle may optionally be mono- to tetra-substituted, identically or differently, by halogen or C₁-C₄-alkyl.
R⁶ and R⁷ independently of one another each particularly preferably represent hydrogen, methyl, ethyl, n- or iso-propyl, n-, iso-, sec- or tert-butyl, methoxymethyl, methoxyethyl, ethoxymethyl, ethoxyethyl, cyclopropyl, cyclopentyl, cyclohexyl; trifluoromethyl, trichloromethyl, trifluoroethyl, trifluoromethoxymethyl.
R⁶ and R⁷ furthermore together with the nitrogen atom to which they are attached, particularly preferably represent a saturated heterocycle selected from the group consisting of morpholine, thiomorpholine and piperazine, which heterocycle may optionally be mono- to tetra-substituted, identically or differently, by fluorine, chlorine, bromine or methyl and where the piperazine additionally at the second nitrogen atom may be substituted by R¹¹.
R⁸ and R⁹ independently of one another each preferably represent hydrogen, C₁-C₆-alkyl, C₃-C₆-cycloalkyl; C₁-C₄-halogenoalkyl, C₃-C₆-halogenocycloalkyl having in each case 1 to 9 fluorine-, chlorine- and/or bromine atoms.
R⁸ and R⁹ furthermore together with the nitrogen atom to which they are attached, preferably represent a saturated 5- to 8-membered heterocycle, which heterocycle may have 1 or 2 additional, non-adjacent heteroatoms selected from the group consisting of oxygen, sulphur and NR¹¹, and which heterocycle may optionally be mono- to tetra-substituted, identically or differently, by halogen or C₁-C₄-alkyl.
R⁸ and R⁹ independently of one another each particularly preferably represent hydrogen, methyl, ethyl, n- or iso-propyl, n-, iso-, sec- or tert-butyl, methoxymethyl, methoxyethyl, ethoxymethyl, ethoxyethyl, cyclopropyl, cyclopentyl, cyclohexyl; trifluoromethyl, trichloromethyl, trifluoroethyl, trifluoromethoxymethyl.
R⁸ and R⁹ furthermore together with the nitrogen atom to which they are attached, particularly preferably represent a saturated heterocycle selected from the group consisting of morpholine, thiomorpholine and piperazine, which heterocycle may optionally be mono- to tetra-substituted, identically or differently, by fluorine, chlorine, bromine or methyl and where the piperazine additionally at the second nitrogen atom may be substituted by R¹¹.
R¹⁰ preferably represents hydrogen, C₁-C₆-alkyl, C₁-C₄-alkoxy, C₁-C₃-alkoxy-C₁-C₃-alkyl, C₃-C₆-cycloalkyl; C₁-C₄-halogenoalkyl, C₁-C₄-halogenoalkoxy, halogeno-C₁-C₃-alkoxy-C₁-C₃-alkyl, C₃-C₆-halogenocycloalkyl having in each case 1 to 9 fluorine-, chlorine- and/or bromine atoms.
R¹⁰ particularly preferably represents hydrogen, methyl, ethyl, n- or iso-propyl, tert-butyl, methoxy, ethoxy, n- or iso-propoxy, tert-butoxy, cyclopropyl; trifluoromethyl, trifluoromethoxy.
R¹¹ preferably represents hydrogen or C₁-C₄-alkyl.
R¹¹ particularly preferably represents hydrogen, methyl, ethyl, n- or iso-propyl, n-, iso-, sec- or tert-butyl.
A preferably represents one of the radicals
A1, A2, A3, A4, A5, A6, A9, A10, A11, A12 or A17.
A particularly preferably represents one of the radicals
A1, A2, A4, A5, A6, A9, A11, A17.
A very particularly preferably represents the radical A1.
A furthermore very particularly preferably represents the radical A2.
A furthermore very particularly preferably represents the radical A4.
A furthermore very particularly preferably represents the radical A5.
A furthermore very particularly preferably represents the radical A6.
A furthermore very particularly preferably represents the radical A9.
A furthermore very particularly preferably represents the radical A11.
A furthermore very particularly preferably represents the radical A17.
R¹² preferably represents hydrogen, cyano, fluorine, chlorine, bromine, iodine, methyl, ethyl, iso-propyl, methoxy, ethoxy, methylthio, ethylthio, cyclopropyl, C₁-C₂-halogenoalkyl, C₁-C₂-halogenoalkoxy each having 1 to 5 fluorine, chlorine and/or bromine atoms, trifluoromethylthio, difluoromethylthio, aminocarbonyl, aminocarbonylmethyl or aminocarbonylethyl.
R¹² particularly preferably represents hydrogen, fluorine, chlorine, bromine, iodine, methyl, ethyl, iso-propyl, monofluoromethyl, monofluoroethyl, difluoromethyl, trifluoromethyl, difluorochloromethyl, trichloromethyl, dichloromethyl, cyclopropyl, methoxy, ethoxy, trifluoromethoxy, trichloromethoxy, methylthio, ethylthio, trifluoromethylthio or difluoromethylthio and
R¹² very particularly preferably represents hydrogen, fluorine, chlorine, bromine, iodine, methyl, iso-propyl, monofluoromethyl, monofluoroethyl, difluoromethyl, trifluoromethyl, difluorochloromethyl or trichloromethyl.
R¹² especially preferably represents methyl, monofluoromethyl, difluoromethyl or trifluoromethyl.
R¹³ preferably represents hydrogen, fluorine, chlorine, bromine, iodine, methyl, ethyl, methoxy, ethoxy, methylthio or ethylthio.
R¹³ particularly preferably represents hydrogen, fluorine, chlorine, bromine, iodine or methyl.
R¹³ very particularly preferably represents hydrogen, fluorine, chlorine or methyl.
R¹⁴ preferably represents hydrogen, methyl, ethyl, n-propyl, iso-propyl, C₁-C₂-halogenoalkyl having 1 to 5 fluorine, chlorine and/or bromine atoms, hydroxymethyl, hydroxyethyl, cyclopropyl, cyclopentyl, cyclohexyl or phenyl.
R¹⁴ particularly preferably represents hydrogen, methyl, ethyl, iso-propyl, trifluoromethyl, di-fluoromethyl, hydroxymethyl, hydroxyethyl or phenyl.
R¹⁴ very particularly preferably represents hydrogen, methyl, trifluoromethyl or phenyl.
R¹⁴ especially preferably represents methyl.
R¹⁵ and R¹⁶ independently of one another each preferably represent hydrogen, fluorine, chlorine, bromine, methyl, ethyl or C₁-C₂-halogenoalkyl having 1 to 5 fluorine, chlorine and/or bromine atoms.
R¹⁵ and R¹⁶ independently of one another each particularly preferably represent hydrogen, fluorine, chlorine, bromine, methyl, ethyl, difluoromethyl, trifluoromethyl, difluorochloromethyl or trichloromethyl.
R¹⁵ and R¹⁶ independently of one another each very particularly preferably represent hydrogen, fluorine, chlorine, bromine, methyl, ethyl, difluoromethyl, trifluoromethyl or trichloromethyl.
R¹⁵ and R¹⁶ especially preferably each represent hydrogen.
R¹⁷ preferably represents fluorine, chlorine, bromine, cyano, methyl, ethyl, C₁-C₂-halogenoalkyl or C₁-C₂-halogenoalkoxy each having 1 to 5 fluorine, chlorine and/or bromine atoms.
R¹⁷ particularly preferably represents fluorine, chlorine, bromine, cyano, methyl, trifluoromethyl, trifluoromethoxy, difluoromethoxy, difluorochloromethoxy or trichloromethoxy.
R¹⁷ very particularly preferably represents fluorine, chlorine, bromine, iodine, methyl, trifluoromethyl or trifluoromethoxy.
R¹⁷ especially preferably represents methyl.
R¹⁸ and R¹⁹ independently of one another each preferably represent hydrogen, fluorine, chlorine, bromine, methyl, ethyl or C₁-C₂-halogenoalkyl having 1 to 5 fluorine, chlorine and/or bromine atoms.
R¹⁸ and R¹⁹ independently of one another each particularly preferably represent hydrogen, fluorine, chlorine, bromine, methyl, ethyl, difluoromethyl, trifluoromethyl, difluorochloromethyl or trichloromethyl.
R¹⁸ and R¹⁹ independently of one another each very particularly preferably represent hydrogen, fluorine, chlorine, bromine, methyl, ethyl, difluoromethyl, trifluoromethyl or trichloromethyl.
R¹⁸ and R¹⁹ especially preferably each represent hydrogen.
R²⁰ preferably represents hydrogen, fluorine, chlorine, bromine, methyl, ethyl or C₁-C₂-halogenoalkyl having 1 to 5 fluorine, chlorine and/or bromine atoms.
R²⁰ particularly preferably represents hydrogen, fluorine, chlorine, bromine, iodine, methyl or trifluoromethyl.
R²⁰ very particularly preferably represents methyl.
R²¹ preferably represents hydrogen, fluorine, chlorine, bromine, iodine, hydroxyl, cyano, C₁-C₄-alkyl, C₁-C₂-halogenoalkyl, C₁-C₂-halogenoalkoxy or C₁-C₂-halogenoalkylthio each having 1 to 5 fluorine, chlorine and/or bromine atoms.
R²¹ particularly preferably represents hydrogen, fluorine, chlorine, bromine, iodine, hydroxyl, cyano, methyl, ethyl, n-propyl, iso-propyl, n-butyl, iso-butyl, sec-butyl, tert-butyl, difluoromethyl, trifluoromethyl, difluorochloromethyl, trichloromethyl, trifluoromethoxy, difluoromethoxy, difluorochloromethoxy, trichloromethoxy, trifluoromethylthio, difluoromethylthio, difluorochloromethylthio or trichloromethylthio.
R²¹ very particularly preferably represents hydrogen, fluorine, chlorine, bromine, iodine, methyl, difluoromethyl, trifluoromethyl or trichloromethyl.
R²¹ especially preferably represents iodine, methyl, difluoromethyl or trifluoromethyl.
R²² preferably represents fluorine, chlorine, bromine, iodine, hydroxyl, cyano, C₁-C₄-alkyl, methoxy, ethoxy, methylthio, ethylthio, difluoromethylthio, trifluoromethylthio, C₁-C₂-halogenoalkyl or C₁-C₂-halogenoalkoxy each having 1 to 5 fluorine, chlorine and/or bromine atoms.

R²² particularly preferably represents fluorine, chlorine, bromine, iodine, hydroxyl, cyano, methyl, ethyl, n-propyl, iso-propyl, n-butyl, iso-butyl, sec-butyl, tert-butyl, trifluoromethyl, difluoromethyl, difluorochloromethyl, trichloromethyl, methoxy, ethoxy, methylthio, ethylthio, difluoromethylthio, trifluoromethylthio, trifluoromethoxy, difluoromethoxy, difluorochloromethoxy or trichloromethoxy.
R²² very particularly preferably represents fluorine, chlorine, bromine, iodine, methyl, trifluoromethyl, difluoromethyl or trichloromethyl.

R²³ preferably represents hydrogen, fluorine, chlorine, bromine, iodine, cyano, C₁-C₄-alkyl, methoxy, ethoxy, methylthio, ethylthio, C₁-C₂-halogenoalkyl or C₁C₂-halogenoalkoxy each having 1 to 5 fluorine, chlorine and/or bromine atoms, C₁-C₂-alkylsulphinyl or C₁-C₂-alkylsulphonyl.
R²³ particularly preferably represents hydrogen, fluorine, chlorine, bromine, iodine, cyano, n-propyl, iso-propyl, n-butyl, iso-butyl, sec-butyl, tert-butyl, trifluoromethyl, difluoromethyl, difluorochloromethyl, trichloromethyl, methoxy, ethoxy, methylthio, ethylthio, trifluoromethoxy, difluoromethoxy, difluorochloromethoxy, trichloromethoxy, methylsulphinyl or methylsulphonyl.
R²³ very particularly preferably represents hydrogen, fluorine, chlorine, bromine, iodine, n-propyl, iso-propyl, n-butyl, iso-butyl, sec-butyl, tert-butyl, trifluoromethyl, difluoromethyl, trichloromethyl, methylsulphinyl or methylsulphonyl.
R²³ especially preferably represents hydrogen.
R²⁴ preferably represents methyl, ethyl or C₁-C₂-halogenoalkyl having 1 to 5 fluorine, chlorine and/or bromine atoms.
R²⁴ particularly preferably represents methyl, ethyl, trifluoromethyl, difluoromethyl, difluorochloromethyl or trichloromethyl.
R²⁵ preferably represents methyl or ethyl.

R²⁵ particularly preferably represents methyl.
Q³ preferably represents a sulphur atom, SO₂ or CH₂.
Q³ particularly preferably represents a sulphur atom or CH₂.
Q³ very particularly preferably represents a sulphur atom.
p preferably represents 0 or 1.
p particularly preferably represents 0.
R²⁶ preferably represents methyl, ethyl or C₁-C₂-halogenoalkyl having 1 to 5 fluorine, chlorine and/or bromine atoms.
R²⁶ particularly preferably represents methyl, ethyl, trifluoromethyl, difluoromethyl, difluorochloromethyl or trichloromethyl.
R²⁶ very particularly preferably represents methyl, trifluoromethyl, difluoromethyl or trichloromethyl.
R²⁷ preferably represents methyl, ethyl or C₁-C₂-halogenoalkyl having 1 to 5 fluorine, chlorine and/or bromine atoms.
R²⁷ particularly preferably represents methyl, ethyl, trifluoromethyl, difluoromethyl, difluorochloromethyl or trichloromethyl.
R²⁷ very particularly preferably represents methyl, trifluoromethyl, difluoromethyl or trichloromethyl.
R²⁸ and R²⁹ independently of one another each preferably represent hydrogen, fluorine, chlorine, bromine, amino, methyl, ethyl or C₁C₂-halogenoalkyl having 1 to 5 fluorine, chlorine and/or bromine atoms.
R²⁸ and R²⁹ independently of one another each particularly preferably represent hydrogen, fluorine, chlorine, bromine, methyl, ethyl, trifluoromethyl, difluoromethyl, difluorochloromethyl or trichloromethyl.
R²⁸ and R²⁹ independently of one another each very particularly preferably represent hydrogen, fluorine, chlorine, bromine, methyl, trifluoromethyl, difluoromethyl or trichloromethyl.
R²⁸ and R²⁹ especially preferably each represent hydrogen.
R³⁰ preferably represents hydrogen, fluorine, chlorine, bromine, iodine, methyl, ethyl or C₁-C₂-halogenoalkyl having 1 to 5 fluorine, chlorine and/or bromine atoms.
R³⁰ particularly preferably represents hydrogen, fluorine, chlorine, bromine, iodine, methyl, ethyl, trifluoromethyl, difluoromethyl, difluorochloromethyl or trichloromethyl.
R³⁰ very particularly preferably represents hydrogen, fluorine, chlorine, bromine, methyl, trifluoromethyl, difluoromethyl or trichloromethyl.
R³⁰ especially preferably represents methyl.
R³¹ and R³² independently of one another each preferably represent hydrogen, fluorine, chlorine, bromine, amino, nitro, methyl, ethyl or C₁-C₂-halogenoalkyl having 1 to 5 fluorine, chlorine and/or bromine atoms.
R³¹ and R³² independently of one another each particularly preferably represent hydrogen, fluorine, chlorine, bromine, nitro, methyl, ethyl, trifluoromethyl, difluoromethyl, difluorochloromethyl or trichloromethyl.
R³¹ and R³² independently of one another each very particularly preferably represent hydrogen, fluorine, chlorine, bromine, methyl, trifluoromethyl, difluoromethyl or trichloromethyl.
R³¹ and R³² especially preferably each represent hydrogen.
R³³ preferably represents hydrogen, fluorine, chlorine, bromine, methyl, ethyl or C₁-C₂-halogenoalkyl having 1 to 5 fluorine, chlorine and/or bromine atoms,
R³³ particularly preferably represents hydrogen, fluorine, chlorine, bromine, methyl, ethyl, trifluoromethyl, difluoromethyl, difluorochloromethyl or trichloromethyl.
R³³ very particularly preferably represents hydrogen, fluorine, chlorine, bromine, methyl, trifluoromethyl, difluoromethyl or trichloromethyl.
R³³ especially preferably represents methyl.
R³⁴ preferably represents hydrogen, fluorine, chlorine, bromine, amino, C₁-C₄-alkyl)amino, di(C₁-C₄-alkyl)amino, cyano, methyl, ethyl or C₁-C₂-halogenoalkyl having 1 to 5 fluorine, chlorine and/or bromine atoms.
R³⁴ particularly preferably represents hydrogen, fluorine, chlorine, bromine, amino, methylamino, dimethylamino, cyano, methyl, ethyl, trifluoromethyl, difluoromethyl, difluorochloromethyl or trichloromethyl.
R³⁴ very particularly preferably represents hydrogen, fluorine, chlorine, bromine, amino, methylamino, dimethylamino, methyl, trifluoromethyl, difluoromethyl or trichloromethyl.
R³⁴ especially preferably represents amino, methylamino, dimethylamino, methyl or trifluoromethyl.

R³⁵ preferably represents fluorine, chlorine, bromine, methyl, ethyl or C₁-C₂-halogenoalkyl having 1 to 5 fluorine, chlorine and/or bromine atoms.
R³⁵ particularly preferably represents fluorine, chlorine, bromine, methyl, ethyl, trifluoromethyl, difluoromethyl, difluorochloromethyl or trichloromethyl.

R³⁵ very particularly preferably represents fluorine, chlorine, bromine, methyl, trifluoromethyl, difluoromethyl or trichloromethyl.
R³⁵ especially preferably represents methyl, trifluoromethyl or difluoromethyl.

R³⁶ preferably represents hydrogen, fluorine, chlorine, bromine, amino, C₁-C₄-alkylamino, di(C₁-C₄-alkyl)amino, cyano, methyl, ethyl or C₁-C₂-halogenoalkyl having 1 to 5 fluorine, chlorine and/or bromine atoms.
R³⁶ particularly preferably represents hydrogen, fluorine, chlorine, bromine, amino, methylamino, dimethylamino, cyano, methyl, ethyl, trifluoromethyl, difluoromethyl, difluorochloromethyl or trichloromethyl.
R³⁶ very particularly preferably represents hydrogen, fluorine, chlorine, bromine, amino, methylamino, dimethylamino, methyl, trifluoromethyl, difluoromethyl or trichloromethyl.
R³⁶ especially preferably represents amino, methylamino, dimethylamino, methyl or trifluoromethyl.
R³⁷ preferably represents fluorine, chlorine, bromine, methyl, ethyl or C₁-C₂-halogenoalkyl having 1 to 5 fluorine, chlorine and/or bromine atoms.
R³⁷ particularly preferably represents fluorine, chlorine, bromine, methyl, ethyl, trifluoromethyl, difluoromethyl, difluorochloromethyl or trichloromethyl.
R³⁷ very particularly preferably represents fluorine, chlorine, bromine, methyl, trifluoromethyl, difluoromethyl or trichloromethyl.
R³⁷ especially preferably represents methyl, trifluoromethyl or difluoromethyl.
R³⁸ preferably represents fluorine, chlorine, bromine, methyl, ethyl or C₁-C₂-halogenoalkyl having 1 to 5 fluorine, chlorine and/or bromine atoms.
R³⁸ particularly preferably represents fluorine, chlorine, bromine, methyl, ethyl, trifluoromethyl, difluoromethyl, difluorochloromethyl or trichloromethyl.
R³⁸ very particularly preferably represents fluorine, chlorine, bromine, methyl, trifluoromethyl, difluoromethyl or trichloromethyl.
R³⁹ preferably represents hydrogen, methyl or ethyl.
R³⁹ particularly preferably represents methyl.
R⁴⁰ preferably represents fluorine, chlorine, bromine, methyl or ethyl,
R⁴⁰ particularly preferably represents fluorine, chlorine or methyl.
R⁴¹ preferably represents methyl, ethyl or C₁-C₂-halogenoalkyl having 1 to 5 fluorine, chlorine and/or bromine atoms.
R⁴¹ particularly preferably represents methyl, ethyl, trifluoromethyl, difluoromethyl, difluorochloromethyl or trichloromethyl.
R⁴¹ very particularly preferably represents methyl, trifluoromethyl, difluoromethyl or trichloromethyl.
R⁴¹ especially preferably represents methyl or trifluoromethyl.
R⁴² preferably represents hydrogen, fluorine, chlorine, bromine, methyl, ethyl or C₁-C₂-halogenoalkyl having 1 to 5 fluorine, chlorine and/or bromine atoms.
R⁴² particularly preferably represents hydrogen, fluorine, chlorine, bromine, methyl or trifluoromethyl.
R⁴³ preferably represents fluorine, chlorine, bromine, iodine, hydroxyl, C₁-C₄-alkyl, methoxy, ethoxy, methylthio, ethylthio, difluoromethylthio, trifluoromethylthio, C₁-C₂-halogenoalkyl or C₁-C₂-halogenoalkoxy each having 1 to 5 fluorine, chlorine and/or bromine atoms.

R⁴³ particularly preferably represents fluorine, chlorine, bromine, iodine, methyl, ethyl, n-propyl, iso-propyl, n-butyl, iso-butyl, sec-butyl, tert-butyl, trifluoromethyl, difluoromethyl, difluorochloromethyl, trichloromethyl.
R⁴³ very particularly preferably represents fluorine, chlorine, bromine, iodine, methyl, trifluoromethyl, difluoromethyl or trichloromethyl.

Moreover, emphasis is given to compounds of the formula (I-I) 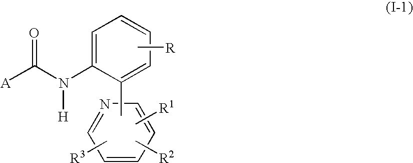
in which R, R¹, R², R³ and A are as defined above,
excluded compounds of the formula (I-1), in which
R represents hydrogen and
R¹, R² and R³ independently of one another each represents hydrogen, halogen;
or represents straight-chain or branched alkyl having 1 to 4 carbon atoms;
or represents straight-chain or branched halogenoalkyl having 1 to 4 carbon atoms;
   and
A represents a radical of the formula (A1) 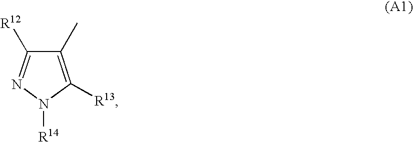
   wherein
R¹² represents halogen, C₁-C₄-alkyl, C₁-C₄-halogenoalkyl and
R¹³ represents hydrogen and
R¹⁴ represents methyl,
   or
A represents a radical of the formula (A2) 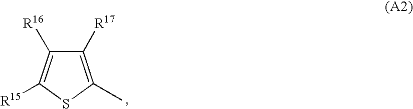
   wherein
R¹⁵ and R¹⁶ independently of one another each represent hydrogen or C₁-C₄-alkyl and
R¹⁷ represents halogen, C₁-C₄-alkyl or C₁-C₄-halogenoalkyl,
   or
A represents a radical of the formula (A4) 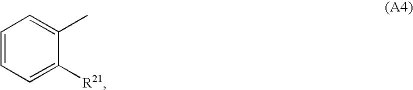
   wherein
R²¹ represents halogen, C₁-C₄-alkyl or C₁-C₄-halogenoalkyl,
   or
A represents a radical of the formula (A5) 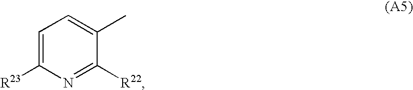
   wherein
R²² represents halogen and
R²³ represents hydrogen,
   or
A represents a radical of the formula (A6) 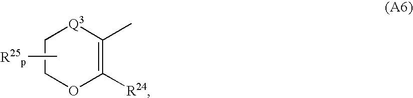
   wherein
R²⁴ represents methyl and
Q³ represents a sulphur or CH₂,
p represents 0,
   or
A represents a radical of the formula (A9) 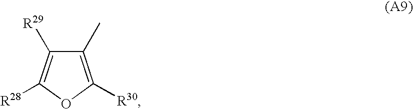
   wherein
R²⁸ and R²⁹ independently of one another each represent hydrogen or C₁-C₄-alkyl and
R³⁰ represents methyl,
   or
A represents a radical of the formula (A11) 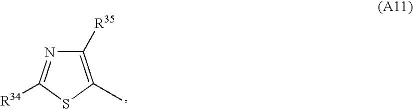
   wherein
R³⁴ represents hydrogen or C₁-C₄-alkyl and
R³⁵ represents halogen, C₁-C₄-alkyl or C₁-C₄-halogenoalkyl,
   or
A represents a radical of the formula (A16) 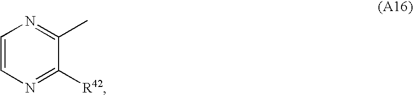
   wherein
R⁴² represents halogen.

Moreover, emphasis is given to compounds of the formula (I-2) 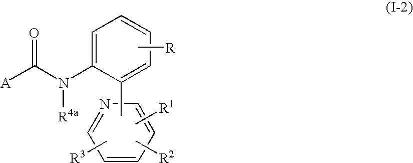
in which R, R¹, R², R³, R⁴a and A are as defined above.
R^{4a} preferably represents C₁-C₆-alkyl, C₁-C₄-alkylsulfinyl, C₁-C₄-alkysulfonyl, C₁-C₃-alkoxy-C₁-C₃-alkyl, C₃-C₆-cycloalkyl; C₁-C₄-halogenoalkyl, C₁-C₄-halogenoalkylthio, C₁-C₄-halogenoalkylsulfinyl, C₁-C₄-halogenoalkysulfonyl, halogeno-C₁-C₃-alkoxy-C₁-C₃-alkyl, C₃-C₆-halogenocycloalkyl each having 1 to 9 fluorine-, chlorine- and/or bromine atoms; formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C₃-halogenoalkyl)carbonyl-C₁-C₃-alkyl, (C₁-C₃-halogenoalkoxy)carbonyl-C₁-C₃-alkyl having in each case 1 to 7 fluorine-, chlorine- and/or bromine atoms, (C₁-C₃-alkyl)carbonyl-C₁-C₃-halogenoalkyl, (C₁-C₃-alkoxy)carbonyl-C₁-C₃-halogenoalkyl having in each case 1 to 6 fluorine-, chlorine- and/or bromine atoms, (C₁-C₃-halogenoalkyl)carbonyl-C₁-C₃-halogenoalkyl, (C₁-C₃-halogenoalkoxy)carbonyl-C₁-C₃-halogenoalkyl having in each case 1 to 13 fluorine-, chlorine- and/or bromine atoms; —COR⁵, —CONR⁶R⁷ or —CH₂NR⁸R⁹, where R⁵, R⁶, R⁷, R⁸ and R⁹ are as defined above.
R^{4a} particularly preferably represents methyl, ethyl, n- or iso-propyl, n-, iso-, sec- or tert-butyl, pentyl or hexyl, methylsulfinyl, ethylsulfinyl, n- or iso-propylsulfinyl, n-, iso-, sec- or tert-butylsulfinyl, methylsulfonyl, ethylsulfonyl, n- or iso-propylsulfonyl, n-, iso-, sec- or tert-butylsulfonyl, methoxymethyl, methoxyethyl, ethoxymethyl, ethoxyethyl, cyclopropyl, cyclopentyl, cyclohexyl, trifluoromethyl, trichloromethyl, trifluoroethyl, difluoromethylthio, difluorochloromethylthio, trifluoromethylthio, trifluoromethylsulfinyl, trifluoromethylsulfonyl, trifluoromethoxymethyl; —CH₂—CHO, —CH₂CH₂—CHO, —CH₂—CO—CH₃, —CH₂—CO—CH₂CH₃, —CH₂—CO—CH(CH₃)₂, —CH₂CH₂—CO—CH₃, —CH₂CH₂—CO—CH₂CH₃, —CH₂CH₂—CO—CH(CH₃)₂, —CH₂—C(O)OCH₃, —CH₂—C(O)OCH₂CH₃, —CH₂—C(O)OCH(CH₃)₂, —CH₂CH₂—C(O)OCH₃, —CH₂CH₂—C(O)OCH₂CH₃, —CH₂CH₂—C(O)OCH(CH₃)₂, —CH₂—CO—CF₃, —CH₂—CO—CCl₃, —CH₂—CO—CH₂CF₃, —CH₂—CO—CH₂CCl₃, —CH₂CH₂—CO—CH₂CF₃, —CH₂CH₂—CO—CH₂CCl₃, —CH₂—C(O)OCH₂CF₃, —CH₂—C(O)OCF₂CF₃, —CH₂—C(O)OCH₂CCl₃, —CH₂—C(O)OCCl₂CCl₃, —CH₂CH₂—C(O)OCH₂CF₃, —CH₂CH₂—C(O)OCF₂CF₃, —CH₂CH₂—C(O)OCH₂CCl₃, —CH₂CH₂—C(O)O—CCl₂CCl₃; —COR⁵, —CONR⁶R⁷ or —CH₂NR⁸R⁹, where R⁵, R⁶, R⁷, R⁸ and R⁹ are as defined above.
R^{4a} very particularly preferably represents methyl, methoxymethyl, —CH₂—CHO, —CH₂CH₂—CHO, —CH₂—CO—CH₃, —CH₂—CO—CH₂CH₃, —CH₂—CO—CH(CH₃)₂ or —COR⁵, where R⁵ is as defined above.

Moreover, emphasis is given to compounds of the formula (I-3) 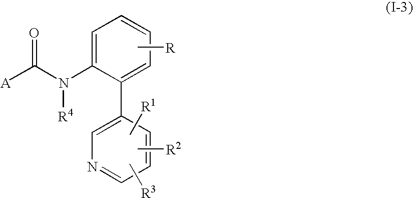
in which R, R¹, R², R³, R⁴ and A are as defined above,
where 2-chloro-N-(2-pyridin-3-ylphenyl)nicotinamide is excluded.

Moreover, emphasis is given to compounds of the formula (I-4) 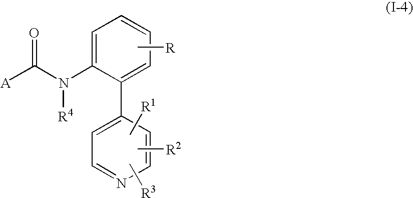
in which R, R¹, R², R³, R⁴ and A are as defined above,
where 2-chloro-N-(2-pyridin-4-ylphenyl)nicotinamide is excluded.

Moreover, emphasis is given to compounds of the formula (I-5) 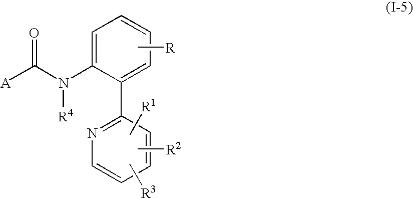
in which R, R¹, R², R³, R⁴ and A are as defined above,
where 2-methyl-N-(2-pyridin-2-ylphenyl)-4-(trifluoromethyl)-1,3-thiazole-5-carboxamide; N-(2-pyridin-2-ylphenyl)-4-(trifluoromethyl)-1,3-thiazole-5-carboxamide; 4-iodo-N-(2-pyridin-2-ylphenyl)-1,3-thiazole-5-carboxamide; 1-methyl-N-(2-pyridin-2-ylphenyl)-3-trifluoromethyl)-1H-pyrazole-4-carboxamide; 3-iodo-1-methyl-N-(2-pyridin-2-ylphenyl)-1H-pyrazole-4-carboxamide; 2-methyl-N-(2-pyridin-2-ylphenyl)-3-furamide; 3-methyl-N-(2-pyridin-2-ylphenyl)thiophene-2-carboxamide; 2-chloro-N-(2-pyridin-2-ylphenyl)benzamide; 2-chloro-N-(2-pyridin-2-ylphenyl)nicotinamide; 3-chloro-N-(2-pyridin-2-ylphenyl)pyrazine-2-carboxamide; 2-methyl-N-(2-pyridin-2-ylphenyl)-5,6-dihydro-1,4-oxathiine-3-carboxamide; 2-methyl-N-[2-(6-methylpyridin-2-yl)phenyl]-4-(trifluoromethyl)-1,3-thiazole-5-carboxamide; 1-methyl-N-[2-(6-methylpyridin-2-yl)phenyl]-3-(trifluoromethyl)-1H-pyrazole-4-carboxamide are excluded.

Moreover, emphasis is given to compounds of the formula (I-6) 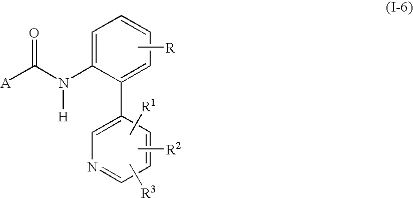
in which R, R¹, R², R³ and A are as defined above,
where 2-chloro-N-(2-pyridin-3-ylphenyl)nicotinamde is excluded.

Moreover, emphasis is given to compounds of the formula (I-7) 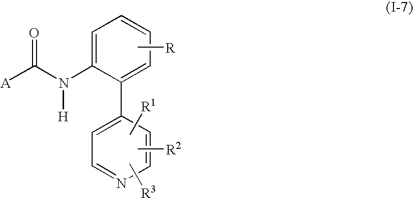
in which R, R¹, R², R³ and A are as defined above,
where 2-chloro-N-(2-pyridin-4-ylphenyl)nicotinamide is excluded.

Moreover, emphasis is given to compounds of the formula (I-8) 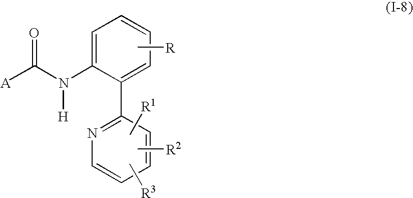
in which R, R¹, R², R³ and A are as defined above,
where 2-methyl-N-(2-pyridin-2-ylphenyl)-4-trifluoromethyl)-1,3-thiazole-5-carboxamide; N-(2-pyridin-2-ylphenyl)-4-(trifluoromethyl)-1,3-thiazole-5-carboxamide; 4iodo-N-(2-pyridin-2-ylphenyl)-1,3-thiazole-5-carboxamide; 1-methyl-N-(2-pyridin-2-ylphenyl)-3-trifluoromethyl)-1H-pyrazole-4-carboxamide; 3-iodo1-methyl-N-(2-pyridin-2-ylphenyl)-1H-pyrazole-4-carboxamide; 2-methyl-N-(2-pyridin-2-ylphenyl)-3-furamide; 3-methyl-N-(2-pyridin-2-ylphenyl)thiophene-2-carboxamide; 2-chloro-N-(2-pyridin-2-ylphenyl)benzamide; 2-chloro-N-(2-pyridin-2-ylphenyl)nicotinamide; 3-chloro-N-(2-pyridin-2-ylphenyl)pyrazine-2-carboxamide; 2-methyl-N-(2-pyridin-2-ylphenyl)-5,6-dihydro-1,4-oxathiine-3-carboxamide; 2-methyl-N-[2-(6-methylpyridin-2-yl)phenyl]-4-(trifluoromethyl)-1,3-thiazole-5-carboxamide; 1-methyl-N-[2-(6-methylpyridin-2-yl)phenyl]-3-(trifluoromethyl)-1H-pyrazole-4-carboxamide are excluded.

Moreover, emphasis is given to compounds of the formula (I-9) 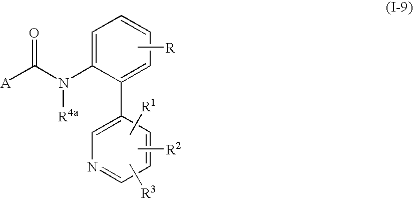
in which R, R¹, R², R³, R^{4a} and A are as defined above.

Moreover, emphasis is given to compounds of the formula (I-10) 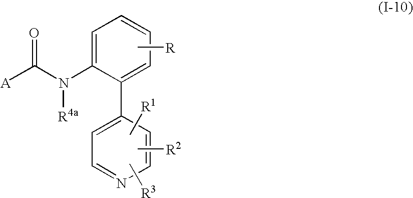
in which R, R¹, R², R³, R^{4a} and A are as defined above.

Moreover, emphasis is given to compounds of the formula (I-11) 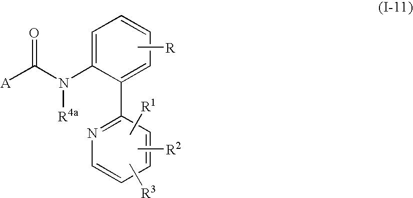
in which R, R¹, R², R³, R^{4a} and A are as defined above.

Moreover, emphasis is given to compounds of the formula (I-12) 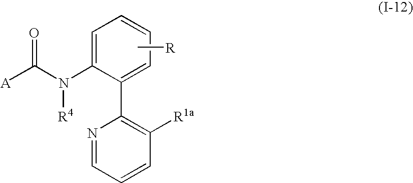
in which R, R⁴ and A are as defined above, and R^{1a} is as defined below.

Moreover, emphasis is given to compounds of the formula (I-13) 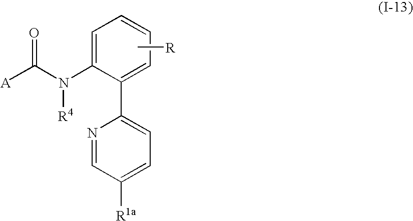
in which R, R⁴ and A are as defined above, and R^{1a} is as defined below.

Moreover, emphasis is given to compounds of the formula (I-14) 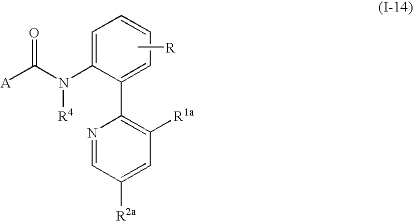
in which R, R⁴ and A are as defined above, and R^{1a} and R^{2a} are as defined below.

Moreover, emphasis is given to compounds of the formula (I-15) 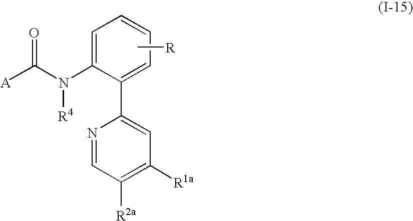
in which R, R⁴ and A are as defined above, and R^{1a} and R^{2a} are as defined below.

Moreover, emphasis is given to compounds of the formula (I-16) 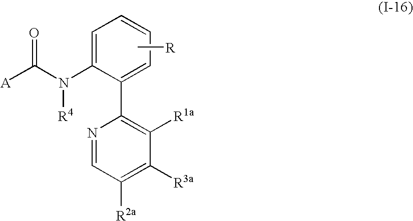
in which R, R⁴ and A are as defined above, and R^{1a}, R^{2a} and R^{3a} are as defined below.
R^{1a}, R^{2a} and R^{3a} independently of one another each represents halogen, cyano, nitro, amino, hydroxyl, formyl, carboxyl, carbamoyl, thiocarbamoyl;
   or represents in each case straight-chain or branched alkyl, hydroxyalkyl, oxoalkyl, alkoxy, alkoxyalkyl, alkylthioalkyl, dialkoxyalkyl, alkylthio, alkylsulfinyl or alkylsulfonyl having in each case 1 to 8 carbon atoms in the respective alkyl moiety;
   or represents in each case straight-chain or branched alkenyl or alkenyloxy having in each case 2 to 6 carbon atoms;
   or represents in each case straight-chain or branched halogenoalkyl, halogenoalkoxy, halogenoalkylthio, halogenoalkylsuyfinyl or halogenoalkylsulfonyl having in each case 1 to 6 carbon atoms and 1 to 13 identical or different halogen atoms;
   or represents in each case straight-chain or branched halogenoalkenyl or halogenoalkenyloxy having in each case 2 to 6 carbon atoms and 1 to 11 identical or different halogen atoms;
   or represents in each case straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, dialkylaminocarbonyloxy having 1 to 6 carbon atoms in the respective hydrocarbon chain, alkenylcarbonyl or alkenylcarbonyl having 2 to 6 carbon atoms in the respective hydrocarbon chain;
   or represents cycloalkyl or cycloalkyloxy having in each case 3 to 6 carbon atoms;
   or represents the grouping —C(Q¹)=N-Q², wherein
      Q¹ represents hydrogen, hydroxyl or C₁-C₄-alkyl, C₁-C₄-halogenoalkyl having 1 to 9 identical or different halogen atoms, or C₃-C₆-cycloalkyl and
      Q² represents hydroxyl, amino, methylamino, phenyl, benzyl; or represents C₁-C₄-alkyl or C₁-C₄-alkoxy, each of which is optionally substituted by halogen, cyano, hydroxyl, C₁-C₄-alkoxy, C₁-C₄-alkylthio, C₁-C₄-alkylamino, di(C₁-C₄-alkyl)amino or phenyl; or represents C₂-C₄-alkenyloxy or C₂-C₄-alkynyloxy,
   represents phenyl, phenoxy, phenylthio, benzoyl, benzoylethenyl, cinnamoyl, heterocyclyl or phenylalkyl, phenylalkyloxy, phenylalkylthio or heterocyclylalkyl having in each case 1 to 3 carbon atoms in the respective alkyl moieties, each of which is optionally mono- to tri-substituted, identically or differently, in the ring moiety by halogen, in each case straight-chain or branched C₁-C₄-alkyl and C₁-C₄-alkoxy.
R^{1a}, R^{2a} and R^{3a} independently of one another each preferably represents halogen, cyano, nitro, amino, hydroxyl, formyl, carboxyl, carbamoyl, thiocarbamoyl;
   or preferably represents in each case straight-chain or branched alkyl, alkoxy, alkoxyalkyl, alkylthioalkyl, alkylthio, alkylsulfinyl or alkylsulfonyl having in each case 1 to 6 carbon atoms;
   or preferably represents in each case straight-chain or branched halogenoalkyl, halogenoalkoxy, halogenoalkylthio, halogenoalkylsulfinyl or halogenoalkylsulfonyl having in each case 1 to 4 carbon atoms and 1 to 9 identical or different halogen atoms;
   or preferably represents in each case straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, dialkylaminocarbonyloxy having 1 to 4 carbon atoms in the respective hydrocarbon chain;
   or preferably represents cycloalkyl or cycloalkyloxy having in each case 3 to 6 carbon atoms;
   or preferably represents the grouping —C(Q¹)=N-Q², wherein
      Q¹ preferably represents hydrogen, hydroxyl or C₁-C₄-alkyl, C₁-C₄-halogenoalkyl having 1 to 9 identical or different halogen atoms or C₃-C₆cycloalkyl and
      Q² preferably represents hydroxyl, C₁-C₄-alkyl, C₁-C₄-alkoxy, C₁-C₄-halogenoalkyl or C₁-C₄-halogenoalkoxy each having 1 to 9 identical or different halogen atoms.
R^{1a}, R^{2a} and R^{3a} independently of one another each particularly preferably represents fluorine, chlorine, bromine, cyano; methyl, ethyl, n- or iso-propyl, n-, iso-, sec- or tert-butyl, methoxy, ethoxy, n- or iso-propoxy, n-, iso-, sec- or tert-butoxy, methylthio, ethylthio, n- or iso-propylthio, n-, iso-, sec- or tert-butylthio, trifluoromethyl, tifluoroethyl, difluoromethoxy, trifluoromethoxy, difluorochloromethoxy, trifluoroethoxy, cyclopropyl, cyclopentyl, cyclohexyl,
or particularly preferably represents the grouping —C(Q¹)=N-Q², wherein
   Q¹ particularly preferably represents hydrogen, methyl, ethyl, trifluoromethyl or cyclopropyl, and
   Q² particularly preferably represents hydroxyl, methoxy, ethoxy, n-propoxy or iso-propoxy.
R^{1a}, R^{2a} and R^{3a} independently of one another each very particularly preferably represents fluorine, chlorine, bromine, iodine, methyl, ethyl, n- or iso-propyl, n-, iso-, sec- or tert-butyl, methoxy, ethoxy, n- or iso-propoxy, trifluoromethyl, trifluoroethyl, difluoromethoxy, trifluoromethoxy, difluorochloromethoxy, trifluoroethoxy,
   or very particularly preferably represents the grouping —C(Q¹)=N-Q², wherein
      Q¹ very particularly preferably represents hydrogen, methyl or ethyl and
      Q² very particularly preferably represents hydroxyl, methoxy, ethoxy, n-propoxy or iso-propoxy.

Saturated or unsaturated hydrocarbon radicals, such as alkyl or alkenyl, can in each case be straight-chain or branched as far as this is possible, even in combination with heteroatoms, such as, for example, in alkoxy.

Optionally substituted radicals can be mono- or poly-substituted, where in the case of poly-substitutions the substituents can be identical or different.

Halogen-substituted radicals, such as, for example, halogenoalkyl, are mono- or poly-halogenated. In the case of poly-halogenation, the halogen atoms can be identical or different. Here, halogen represents fluorine, chlorine, bromine and iodine, in particular fluorine, chlorine and bromine.

However, the general or preferred radical definitions or illustrations listed above can also be combined with one another as desired, i.e. including combinations between the respective ranges and preferred ranges. They apply to the end products and, correspondingly, to precursors and intermediates. Moreover, individual definitions may not apply.

### DETAILED DESCRIPTION OF THE PROCESSES AND INTERMEDIATES

Process (a)

Using 2-(trifluoromethyl)benzoyl chloride and 2-[3-fluoro-5-(trifluoromethyl)-2-pyridinyl]phenylamine as starting materials, the course of the process (a) according to the invention can be illustrated by the formula scheme below.

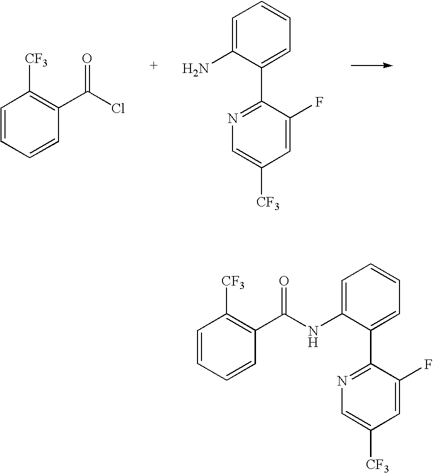

The formula (II) provides a general definition of the carboxylic acid derivatives required as starting materials for carrying out the process (a) according to the invention. In this formula, A preferably has those meanings which have already been mentioned in connection with the description of the compounds of the formula (I) according to the invention as being preferred, particularly preferred or very particularly preferred for these radicals. X¹ preferably represents chlorine, bromine or hydroxyl, particularly preferably chlorine or hydroxyl.

The carboxylic acid derivatives of the formula (II) are known or can be prepared by known processes (cf. WO 93/11117, EP-A 0 545 099, EP-A 0 589 301 and EP-A 0 589 313).

The formula (III) provides a general definition of the amines required as reaction components for carrying out the process (a) according to the invention. In this formula, R, R¹, R² and R³ preferably have those meanings which have already been mentioned in connection with the description of the compounds of the formula (I) according to the invention as being preferred, particularly preferred or very particularly preferred for these radicals.

The amines of the formula (III) excluded compounds of the formula (III), in which R represents hydrogen and R¹, R² and R³ independently of one another each represents hydrogen, halogen, straight-chain or branched alkyl having 1 to 4 carbon atoms or straight-chain or branched halogenoalkyl having 1 to 4 carbon atoms; and R⁴ represents hydrogen are novel (cf. JP-A 8-92223). Amines of the formula (III) employed for the production of compounds of the formulae (I-12), (I-13), (I-14), (I-15) and (I-16) are novel as well. Some of them can be prepared by known methods (Heterocycles 1989, 29, 1013-1016; J. Med. Chem. 1996, 39, 892-903; Synthesis 1995, 713-16; Synth. Commun. 1994 24, 267-272; DE-A 27 27 416; Synthesis 1994 142-144; EP-A 0 824 099; WO 93/11117, EP-A 0 545 099, EP-A 0 589 301, EP-A 0 589 313 and WO 02/38542).

Moreover, aniline derivatives of the formula (M) are obtained by

f) reacting 2-halo-amines of the general formula (IX) 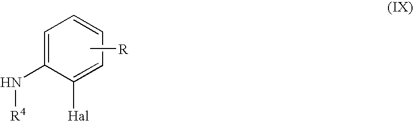
in which
R and R⁴ are as defined above and
Hal represents halogen,
with boronic acid derivatives of the formula (V) 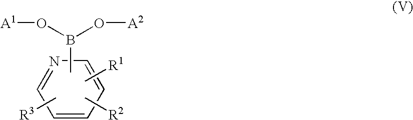
in which R¹, R², R³, A¹ and A² are as defined above,
if appropriate in the presence of an acid binder, and if appropriate in the presence of an inert organic diluent, and if appropriate in the presence of a catalyst,
   or

g) reacting boronic acid derivatives of the formula (X) 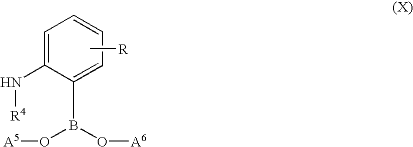
in which
R and R⁴ are as defined above, and
A⁵ and A⁶ each represent hydrogen or together represent tetramethylethylene,
with pyridinyl derivatives of the formula (VII) 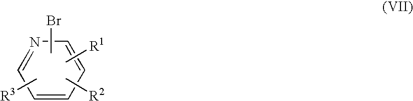
in which R¹, R² and R³ are as defined above,
if appropriate in the presence of an acid binder, and if appropriate in the presence of an inert organic diluent, and if appropriate in the presence of a catalyst,
   or

h) reacting 2-halo-amines of the general formula (IX) 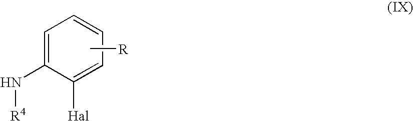
in which
R and R⁴ are as defined above and
Hal represents halogen,
with pyridinyl derivatives of the formula (VII) 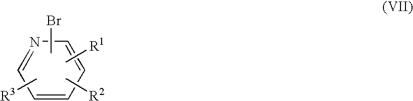
in which R¹, R² and R³ are as defined above,
in the presence of a palladium or platinum catalyst and in the presence of 4,4,4′,4′,5,5,5′,5′-octamethyl-2,2′-bis-1,3,2-dioxaborolane, if appropriate in the presence of an acid binder and if appropriate in the presence of a diluent.

The formula (IX) provides a general definition of the 2-halo-amines required as reaction components for carrying out the processes (f) and (h) according to the invention. In this formula R and R⁴ preferably have those meanings which have already been mentioned in connection with the description of the compounds of the formula (I) according to the invention as being preferred, particularly preferred or very particularly preferred for these radicals. Hal preferably represents chlorine, bromine or iodine, particularly preferably bromine or iodine.

2-Halo-amines of the formula (IX) are known and/or can be prepared by known methods from the corresponding nitro compounds by reduction. In the case, that R⁴ does not represent hydrogen, the compounds of formula (IX) can be obtained by known derivatizations of the resulting aniline derivatives.

The boronic acid derivatives of the formula (V) furthermore required as starting materials for carrying out the process (f) according to the invention are described in more detail below in connection with the process (b) according to the invention.

The formula (X) provides a general definition of the boronic acid derivatives required as reaction components for carrying out the process (g) according to the invention. In this formula R and R⁴ preferably have those meanings which have already been mentioned in connection with the description of the compounds of the formula (I) according to the invention as being preferred, particularly preferred or very particularly preferred for these radicals. A⁵ and A⁶ preferably each represent hydrogen or together represent tetramethylethylene.

The boronic acid derivatives of the formula (X) are known and/or can be obtained by known methods.

The phenyl derivatives of the formula (VII) furthermore required as starting materials for carrying out the processes (g) and (h) according to the invention are illustrated in more detail below, in connection with the process (c) according to the invention.

Process (b)

Using N-(2-bromophenyl)-3-difluoromethyl)-1-methyl-1H-pyrazole-4-carboxamide and 2-chloro-5-(4,4,5,5-tetramethyl-1,3,2-dioxaborolan-2-yl)pyridine as starting materials and a catalyst, the course of the process (b) according to the invention can be illustrated by the formula scheme below.

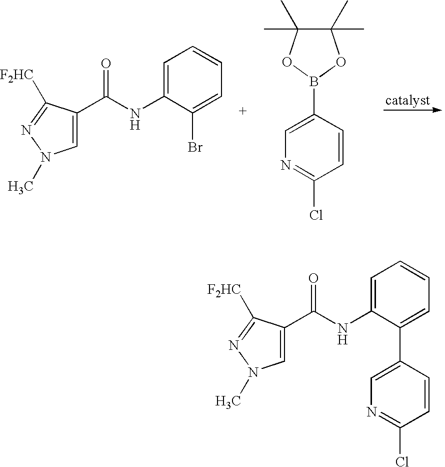

The formula V provides a general definition of the halogeno-carboxamides required as starting materials for carrying out the process (b) according to the invention. In this formula R, R⁴ and A preferably have those meanings which have already been mentioned in connection with the description of the compounds of the formula (I) according to the invention as being preferred, particularly preferred or very particularly preferred for these radicals.

The carboxamide derivatives of the formula (IV) are known or can be prepared by known processes (cf. WO 91/01311, EP-A 0 371 950). They are obtained, for example, by

i) reacting carboxylic acid derivatives of the formula (II) 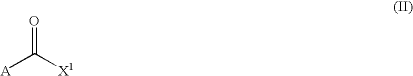
in which
X¹ represents halogen or hydroxyl and
A is as defined above,
with 2-halo-amines of the general formula (IX) 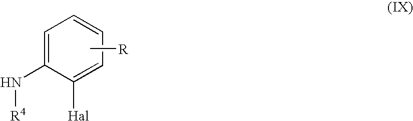
in which
R and R⁴ are as defined above and
Hal represents halogen,
if appropriate in the presence of a catalyst, if appropriate in the presence of a condensing agent, if appropriate in the presence of an acid binder and if appropriate in the presence of a diluent.

The carboxylic acid derivatives of the formula (III) required as starting materials for carrying out the process (i) according to the invention are illustrated in more detail above, in connection with the process (a) according to the invention.

The 2-halo-amines of the formula (IX) furthermore required as starting materials for carrying out the process (i) according to the invention are illustrated in more detail above, in connection with the process (f) according to the invention.

The formula (V) provides a general definition of the boronic acid derivatives furthermore required as starting materials for carrying out the process (b) according to the invention. In this formula R¹. R² and R³ preferably have those meanings which have already been mentioned in connection with the description of the compounds of the formula (I) according to the invention as being preferred, particularly preferred or very particularly preferred for these radicals. A¹ and A² preferably each represent hydrogen or together represent tetramethylethylene.

The boronic acid derivatives of the formula (V) are known and/or can be prepared by known processes (cf. WO 01/90084 and U.S. Pat. No. 5,633,218). They are obtained, for example, by

k) reacting pyridinyl derivatives of the formula (VII) 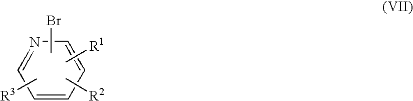
in which R¹, R² and R³ are as defined above,
with boric acid esters of the formula (XI)
   B(O-Alk)₃ (XI)
in which Alk represents C₁-C₄-alkyl,
or with 4,4,4′,4′,5,5,5′,5′-octamethyl-2,2′-bis-1,3,2-dioxaborolane
in the presence of magnesium or alkyllithium, if appropriate in the presence of a diluent (for example tetrahydrofuran).

The formula (XI) provides a general definition of the boric acid esters required as reaction components for carrying out the process (h) according to the invention. In this formula, Alk preferably represents methyl, ethyl, n- or iso-propyl, particularly preferably methyl or ethyl.

The boric acid esters of the formula (XI) are known chemicals for synthesis.

The pyridinyl derivatives of the formula (VII) furthermore required as starting materials for carrying out the process (h) according to the invention are illustrated in more detail below, in connection with the process (c) according to the invention.

Process (c)

Using 2-{[(3-methyl-2-thienyl)carbonyl]amino}phenylboronic acid and 2-bromo-3chloro-5-(trifluoromethyl)pyridine as starting materials and a catalyst, the course of the process (c) according to the invention can be illustrated by the formula scheme below. 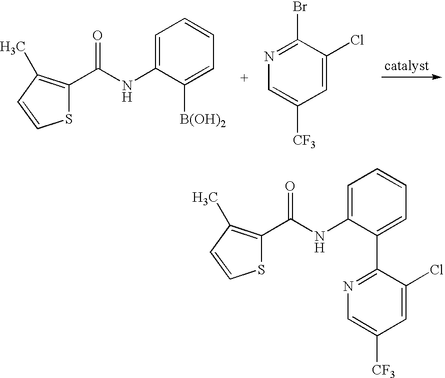
The formula (VI) provides a general definition of the carboxamide boronic acid derivatives required as reaction components for carrying out the process (c) according to the invention. In this formula R, R⁴ and A preferably have those meanings which have already been mentioned in connection with the description of the compounds of the formula (I) according to the invention as being preferred, particularly preferred or very particularly preferred for these radicals. A³ and A⁴ preferably each represent hydrogen or together represent tetramethylethylene.

The carboxamide boronic acid derivatives of the formula (VI) are known and/or can be prepared by known processes.

The formula (VII) provides a general definition of the pyridinyl derivatives required as starting materials for carrying out the process (c) according to the invention. In this formula R¹, R² and R³ preferably have those meanings which have already been mentioned in connection with the description of the compounds of the formula (I) according to the invention as being preferred, particularly preferred or very particularly preferred for these radicals.

The pyridinyl derivatives of the formula (VII) are known or can be prepared by known processes (cf. Synth. Commun. 2000, 30, 665-669, Synth. Commun. 1999, 29, 1697-1701, and cf. also the examples below).

Process (d)

Using N-(2-bromophenyl)-2-chloronicotinamide and 2-bromo-5-chloropyridine as starting materials and a catalyst and 4,4,4′,4′,5,5,5′,5′-octamethyl-2,2′-bis-1,3,2-dioxaborolane, the course of the process (d) according to the invention can be illustrated by the formula scheme below.

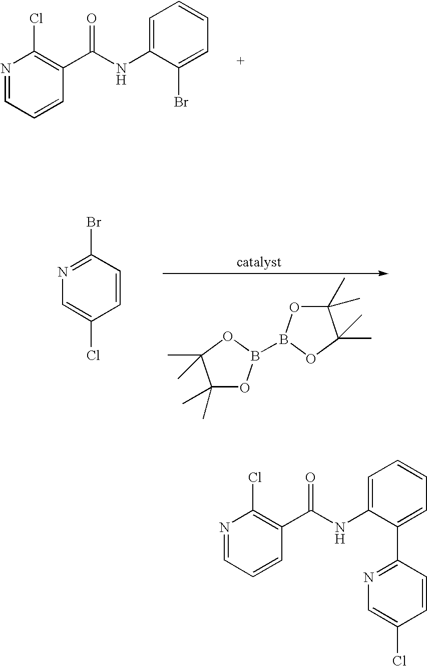

The halogeno-carboxamides of the formula (IV) and the pyridinyl derivatives of the formula (VII) required as starting materials for carrying out the process (d) according to the invention are already described above in connection with the processes (b) and (c) according to the invention.

4,4,4′,4′,5,5,5′,5′-Octamethyl-2,2′-bis-1,3,2-dioxaborolane furthermore required for carrying out process (d) according to the invention is a known chemical substance.

Process (e)

Using N-[2-(5-chloro-2-pyridinyl)phenyl)-5-fluoro-1,3,2-dimethyl-1H-pyrazole-4-carboxamide and acetyl chloride as starting materials, the course of the process (e) according to the invention can be illustrated by the formula scheme below.

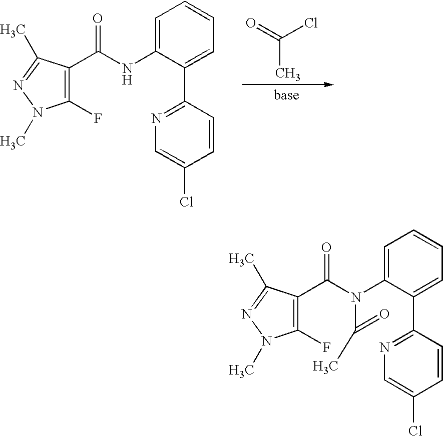

The formula (I-1) provides a general definition of the pyridinylanilides required as starting materials for carrying out the process (e) according to the invention. In this formula R, R¹, R², R³ and A preferably have those meanings which have already been mentioned in connection with the description of the compounds of the formula (I) according to the invention as being preferred, particularly preferred or very particularly preferred for these radicals.

The compounds of the formula (I-1) are compounds according to the invention and can be obtain according to any of the processes (a) to (d).

The formula (VIII) provides a general definition of the halogenides required as starting materials for carrying out the process (e) according to the invention. In this formula R^{4a} preferably has those meanings which have already been mentioned in connection with the description of the compounds of the formula (I-2) according to the invention as being preferred, particularly preferred or very particularly preferred for these radicals. X³ represents chlorine, bromine or iodine.

Halogenides of the formula (VIII) are widely known.

Reaction Conditions

Suitable diluents for carrying out the processes (a) and (i) according to the invention are all customary inert organic solvents. Preference is given to using aliphatic, alicyclic or aromatic hydrocarbons, such as petroleum ether, hexane, heptane, cyclohexane, methylcyclohexane, benzene, toluene, xylene or decalin; halogenated hydrocarbons, such as chlorobenzene, dichlorobenzene, dichloromethane, chloroform, carbon tetrachloride, dichloroethane or trichloroethane; ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisole; nitriles, such as acetonitrile, propionitrile, n- or iso-butyronitrile or benzonitrile; amides, such as N,N-dimethylformamide, N,N-dimethylacetamide, N-methylformanilide, N-methylpyrrolidone or hexamethylphosphoric triamide; mixtures thereof with water or pure water.

Suitable diluents for carrying out the processes (b), (c), (d), (f), (g) and (h) according to the invention are in each case all customary inert organic solvents. Preference is given to using aliphatic, alicyclic or aromatic hydrocarbons, such as petroleum ether, hexane, heptane, cyclohexane, methylcyclohexane, benzene, toluene, xylene or decalin; ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisole; nitriles, such as acetonitrile, propionitrile, n- or iso-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethylsulphoxide; or sulphones, such as sulpholane; alcohols, such as methanol, ethanol, n- or iso-propanol, n-, iso-, sec- or tert-butanol, ethanediol, propane-1,2-diol, ethoxyethanol, methoxyethanol, diethyleneglycolmonomethylether, diethyleneglycolmonoethylether, mixtures thereof with water or pure water.

Suitable diluents for carrying out the process (e) according to the invention are all customary inert organic solvents. Preference is given to using aliphatic, alicyclic or aromatic hydrocarbons, such as petroleum ether, hexane, heptane, cyclohexane, methylcyclohexane, benzene, toluene, xylene or decalin; halogenated hydrocarbons, such as chlorobenzene, dichlorobenzene, dichloromethane, chloroform, carbon tetrachloride, dichloroethane or trichloroethane; ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisole; amides, such as N,N-dimethylformamide, N,N-dimethylacetamide, N-methylformanilide, N-methylpyrrolidone or hexamethylphosphoric triamide.

Suitable acid binders for carrying out the processes (a) and (i) according to the invention are all inorganic and organic bases customary for such reactions. Preference is given to using alkaline earth metal or alkali metal hydrides, hydroxides, amides, alcoholates, acetates, carbonates or hydrogen carbonates, such as sodium hydride, sodium amide, lithiium diisoproylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, or ammonium carbonate; and also tertiary amines, such as trimethylamine, triethylamine, tributylamine, N,N-dimethylaniline, N,N-dimethyl-benzylamine pyridine, N-methylpiperidine, N-methylmorpholine, N,N-dimethylaminopyridine, diazabicyclooctane (DABCO), diazabicyclononene (DBN) or di-azabicycloundecene (DBU).

Suitable acid binders for carrying out the processes (b), (c), (d), (f), (g) and (h) according to the invention are in each case all inorganic and organic bases customary for such reactions. Preference is given to using alkaline earth metal or alkali metal hydrides, hydroxides, amides, alcoholates, acetates, fluorides, phosphates, carbonates or hydrogen carbonates, such as sodium hydride, sodium amide, lithium diisopropylamide, sodium methanolate, sodium ethanolate, potassium tert-butanolate, sodium hydroxide, potassium hydroxide, sodium acetate, sodium phosphate, potassium phosphate, potassium fluoride, caesium fluoride, sodium carbonate, potassium carbonate, potassium hydrogencarbonate, sodium hydrogencarbonate or caesium carbonate; and also tertiary amines, such as trimethylamine, triethylamine, tributylamine, N,N-dimethylaniline, N,N-dimethyl-benzylamine, pyridine, N-methylpiperidine, N-methylmorpholine, N,N-methylaminopyridine, diazabicyclooctane (DABCO), diazabicyclononene (DBN) or diazabicycloundecene (DBU).

Suitable acid binders for carrying out the process (e) according to the invention are all inorganic and organic bases customary for such reactions. Preference is given to using alkaline earth metal or alkali metal hydrides, hydroxides, amides, alcoholates, acetates, carbonates or hydrogen carbonates, such as sodium hydride, sodium amide, lithiium diisoproylamide, sodium methanolate, sodium ethanolate, potassium tert-butanolate, sodium acetate, potassium acetate, calcium acetate, ammonium acetate, sodium hydroxide, potassium hydroxide, ammonium hydroxide, sodium carbonate, potassium carbonate, potassium bicarbonate, sodium bicarbonate, or caesium carbonate; and also tertiary amines, such as trimethylamine, triethylamine, tributylamine, N,N-dimethylaniline, N,N-dimethylbenzylamine pyridine, N-methylpiperidine, N-methylmorpholine, N,N-dimethylaminopyridine, diazabicyclooctane (DABCO), diazabicyclononene (DBN) or diazabicycloundecene (DBU).

Suitable condensing agents for carrying out the processes (a) and (i) according to the invention are all condensing agents customary for such amidation reactions. Preference is given to using acid halide former, such as phosgene, phosphorous tribromide, phosphorous trichloride, phosphorous—pentachloride, phosphorous trichloride oxide or thionyl chloride; anhydride former, such as ethyl chloroformate, methyl chloroformate, isopropyl chloroformate, isobutyl chloroformate or methanesulfonyl chloride; carbodiimides, such as N,N′-dicyclohexylcarbodiimide (DCC) or other customary condensing agents, such as phosphorous pentoxide, polyphosphoric acid, N,N′-carbonyldiimidazole, 2-ethoxy-N-ethoxycarbonyl-1,2-dihydroquinoline (EEDQ), triphenylphosphine/tetrachloromethane or bromo-tripyrrolidinophosphonium-hexafluorophosphate.

The processes (a) and (i) according to the invention is optionally carried out in the presence of a catalyst. Preference is given to 4-dimethylaminopyridine, 1-hydroxy-benzotriazole or dimethylformamide.

The processes (b), (c), (d), (f), (g) and (h) according to the invention are carried out in the presence of a catalyst. Preference is given to palladium salts or complexes, such as palladium chloride, palladium acetate, tetrakis-(triphenylphosphine) palladium, bis-(triphenylphosphine) palladium dichloride or 1,1′-Bis(diphenylphosphino)ferrocenepalladium(II)chloride.

It is also possible to generate a palladium complex directly in the reaction mixture by separately adding to the reaction mixture a palladium salt and a complex ligand, such as triethylphosphane, tri-tert-butylphosphane, tricyclohexylphosphane, 2-(dicyclohexylphosphane)biphenyl, 2-(di-tert-butyl-phosphan)biphenyl, 2-(dicyclohexylphosphane)-2′-(N,N-dimethylamino)-biphenyl, triphenylphosphane, tris-(o-tolyl)phosphane, sodium 3-(diphenylphosphino)benzolsulfonate, tris-2-methoxyphenyl)phosphane, 2,2′-bis-diphenylphosphane)-1,1′-binaphthyl, 1,4-bis-diphenylphosphane)butane, 1,2-bis-diphenylphosphane)ethane, 1,4-bis-(dicyclohexylphosphane)butane, 1,2-bis-(dicyclohexylphosphane)ethane, 2-dicyclohexylphosphane)-2′-(N,N-dimethylamino)-biphenyl, bis(diphenylphosphino)ferrocene or tris-(2,4-tert-butylphenyl)-phosphite.

When carrying out the processes (a) and (i) according to the invention, the reaction temperature can be varied within a relatively wide range. In general, the process is carried out at temperatures between 0° C. and 150° C., preferably between 0° C. and 120° C., particularly preferably between 10° C. and 80° C.

When carrying out the processes (b), (c), (d), (f), (g) and (h) according to the invention, the reaction temperatures can in each case be varied within a relatively wide range. In general, the processes are carried out at temperatures between 0° C. and 180° C., preferably between 10° C. and 150° C., particularly preferably between 20° C. and 120° C.

When carrying out the process (e) according to the invention, the reaction temperature can be varied within a relatively wide range. In general, the process is carried out at temperatures between 0° C. and 150° C., preferably between 20° C. and 110° C.

When carrying out the process (a) according to the invention, in general between 0.8 and 15 mole, preferably of between 0.8 and 8 mole, of amine of the formula (III) and from 1 to 3 mole of acid binder are employed per mole of carboxylic acid derivative of the formula (II). However, it is also possible to employ the reaction components in other ratios. Work-up is carried out by customary methods. In general, water is added to the reaction mixture and the organic phase is separated off and, after drying, concentrated under reduced pressure. The residue that remains may, if appropriate, be freed of any impurities that may still be present using customary methods, such as chromatography or recrystallization.

When carrying out the process (b) according to the invention, in general 1 to 15 mole, preferably from 2 to 8 mole, of boronic acid derivative of the formula (V) and from 1 to 5 mol of acid binder are employed per mole of halogeno-carboxamide of the formula (IV). However, it is also possible to employ the reaction components in other ratios. Work-up is carried out by customary methods. In general, water is added to the reaction mixture and the precipitate is separated off and dried. The residue that remains may, if appropriate, be freed of any impurities that may still be present using customary methods, such as chromatography or recrystallization.

When carrying out the process (c) according to the invention, in general 0.8 to 15 mole, preferably from 0.8 to 8 mole, of pyridinyl derivative of the formula (VII) and from 1 to 10 mol of acid binder and from 0.5 to 5 mole % of a catalyst are employed per mole of carboxamide boronic acid derivative of the formula (VI). However, it is also possible to employ the reaction components in other ratios. Work-up is carried out by customary methods. In general, water is added to the reaction mixture and the precipitate is separated off and dried. The residue that remains may, if appropriate, be freed of any impurities that may still be present using customary methods, such as chromatography or recrystallization.

When carrying out the process (d) according to the invention, in general 0.8 to 15 mole, preferably from 0.8 to 8 mole, of pyridinyl derivative of the formula (VII) and from 0.8 to 15 mole, preferably from 0.8 to 8 mole, of 4,4,4′,4′,5,5,5′,5′-octamethyl-2,2′-bis-1,3,2-dioxaborolane and from 1 to 5 mol of acid binder and from 1 to 5 mol of a catalyst are employed per mole of carboxamide derivative of the formula (IV). However, it is also possible to employ the reaction components in other ratios. Work-up is carried out by customary methods. In general, water is added to the reaction mixture and the precipitate is separated off and dried. The residue that remains may, if appropriate, be freed of any impurities that may still be present using customary methods, such as chromatography or recrystallization.

When carrying out process (e) according to the invention, per mole of the pyridinylanilide of the formula (I-1) in general 0.2 to 5 mole, preferably 0.5 to 2 mole of an halogenide of the formula (VII) are employed. However, it is also possible to employ the reaction components in other ratios. Work-up is carried out by customary methods.

All processes according to the invention are generally each carried out under atmospheric pressure. However, in each case it is also possible to operate under elevated or reduced pressure—in general between 0,1 bar and 10 bar.

The substances according to the invention have potent microbicidal activity and can be employed for controlling unwanted micro-organisms, such as fungi and bacteria, in crop protection and in the protection of materials.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:
Xanthomonas species, such as, for example, Xanthomonas campestris pv. oryzae;
Pseudomonas species, such as, for example, Pseudomonas syringae pv. lachrymans;
Erwinia species, such as, for example, Erwinia amylovora;
Pythium species, such as, for example, Pythium ultimum;
Phytophthora species, such as, for example, Phytophthora infestans;
Pseudoperonospora species, such as, for example, Pseudoperonospora humuli or Pseudoperonospora cubensis;
Plasmopara species, such as, for example, Plasmopara viticola;
Bremia species, such as, for example, Bremia lactucae;
Peronospora species, such as, for example, Peronospora pisi or P. brassicae;
Erysiphe species, such as, for example, Erysiphe graminis;
Sphaerotheca species, such as, for example, Sphaerotheca fuliginea;
Podosphaera species, such as, for example, Podosphaera leucotricha;
Venturia species, such as, for example, Venturia inaequalis;
Pyrenophora species, such as, for example, Pyrenophora teres or P. graminea(conidia form: Drechslera, syn: Helminthosporium);
Cochliobolus species, such as, for example, Cochliobolus sativus (conidia form: Drechslera, syn: Helminthosporium);
Uromyces species, such as, for example, Uromyces appendiculatus;
Puccinia species, such as, for example, Puccinia recondita;
Sclerotinia species, such as, for example, Sclerotinia sclerotiorum;
Tilletia species, such as, for example, Tilletia caries;
Ustilago species, such as, for example, Ustilago nuda or Ustilago avenae;
Pellicularia species, such as, for example, Pellicularia sasakii;
Pyricularia species, such as, for example, Pyricularia oryzae;
Fusarium species, such as, for example, Fusarium culmorum;
Botrytis species, such as, for example, Botrytis cinerea;
Septoria species, such as, for example, Septoria nodorum;
Leptosphaeria species, such as, for example, Leptosphaeria nodorum;
Cercospora species, such as, for example, Cercospora canescens;
Alternaria species, such as, for example, Alternaria brassicae; and
Pseudocercosporella species, such as, for example, Pseudocercosporella herpotrichoides.

The active compounds according to the invention also have very good fortifying action in plants. Accordingly, they can be used for mobilizing the defences of the plant against attack by unwanted micro-organisms.

In the present context, plant-fortifying (resistance-inducing) substances are to be understood as meaning those substances which are capable of stimulating the defence system of plants such that, when the treated plants are subsequently inoculated with unwanted micro-organisms, they show substantial resistance against these micro-organisms.

In the present case, unwanted micro-organisms are to be understood as meaning phytopathogenic fungi, bacteria and viruses. Accordingly, the substances according to the invention can be used to protect plants for a certain period after the treatment against attack by the pathogens mentioned. The period for which protection is provided generally extends over 1 to 10 days, preferably 1 to 7 days, after the treatment of the plants with the active compounds.

The fact that the active compounds are well tolerated by plants at the concentrations required for controlling plant diseases permits the treatment of above-ground parts of plants, of propagation stock and seeds, and of the soil.

The active compounds according to the invention are also suitable for increasing the yield of crops. In addition, they show reduced toxicity and are well tolerated by plants.

At certain concentrations and application rates, the active compounds according to the invention can also be used as herbicides, for influencing plant growth and for controlling animal pests. They can also be used as intermediates and precursors for the synthesis of further active compounds.

The active compounds according to the invention can be used to treat all plants and parts of plants. By plants are understood here all plants and plant populations such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including the plant varieties which can or cannot be protected by plant varieties property rights. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested plants and vegetative and generative propagation material, for example seedlings, tubers, rhizomes, cuttings and seeds.

The treatment of the plants and the parts of plants with the active compounds according to the invention is carried out directly or by action on their surroundings, habitat or storage space, according to customary treatment methods, for example by dipping, spraying, evaporating, atomizing, broadcasting, spreading-on and, in the case of propagation material, in particular in the case of seeds, furthermore by one- or multi-layer coating.

In the protection of materials, the compounds according to the invention can be employed for protecting industrial materials against infection with, and destruction by, undesired micro-organisms.

Industrial materials in the present context are understood as meaning non-living materials which have been prepared for use in industry. For example, industrial materials which are intended to be protected by active compounds according to the invention from microbial change or destruction can be adhesives, sizes, paper and board, textiles, leather, wood, paints and plastic articles, cooling lubricants and other materials which can be infected with, or destroyed by, micro-organisms. Parts of production plants, for example cooling-water circuits, which may be impaired by the proliferation of micro-organisms may also be mentioned within the scope of the materials to be protected. Industrial materials which may be mentioned within the scope of the present invention are preferably adhesives, sizes, paper and board, leather, wood, paints, cooling lubricants and heat-transfer liquids, particularly preferably wood.

Micro-organisms capable of degrading or changing the industrial materials which may be mentioned are, for example, bacteria, fungi, yeasts, algae and slime organisms. The active compounds according to the invention preferably act against fungi, in particular moulds, wood-discolouring and wood-destroying fungi (Basidiomycetes), and against slime organisms and algae.

Micro-organisms of the following genera maybe mentioned as examples:
Alternaria, such as Alternaria tenuis,
Aspergillus, such as Aspergillus niger,
Chaetomium, such as Chaetomium globosum,
Coniophora, such as Coniophora puetana,
Lentinus, such as Lentinus tigrinus,
Penicillium, such as Penicillium glaucum,
Polyporus, such as Polyporus versicolor,
Aureobasidium, such as Aureobasidium pullulans,
Sclerophoma, such as Sclerophoma pityophila,
Trichoderma, such as Trichoderma viride,
Escherichia, such as Escherichia coli,
Pseudomonas, such as Pseudomonas aeruginosa, and
Staphylococcus, such as Staphylococcus aureus.

Depending on their particular physical and/or chemical properties, the active compounds can be converted to the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is, liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk quartz, attapulgite, montmorillonite or diatomaceous earth, and ground synthetic minerals such as finely divided silica, alumina and silicates. Suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention can be used as such or in their formulations, also in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Examples of suitable mixing components are the following:

Fungicides:

2-phenylphenol; 8-hydroxychinolinsulfat; acibenzolar-S-methyl; aldimorph; amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamin; calcium polysulfide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetyl-sodium; fuberidazole; furalaxyl; furametpyr, furcarbanil; furmecyclox; guazatine; hexachlorobenzene; hexaconazole; hymexazol; imazalil; imibenconazole; iminoctadine triacetate; iminoctadine tris(albesil; iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-m; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazol; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolnitrine; quinconazole; quinoxyfen; quintozene; simeconazole; spiroxamine; sulfur, tebuconazole; tecloftalam; tecnazene; tetcyclacis; tetraconazole; thiabendazole; thicyofen; thifluzamide; thiophanate-methyl; thiram; tioxymnid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin a; vinclozolin; zineb; ziram; zoxamide; (2S)—N-[2-[4-[[3-(-chlorophenyl)-2-propynyl]oxy]-3-methoxyphenyl]ethyl]-3-methyl-2-[(methylsulphonyl)amino]butanamide; 1-(1-naphthalenyl)-1H-pyrrole-2,5-dione; 2,3,5,6-tetrachloro(methylsulphonyl)pyridine; 2-amino-4-methyl-N-phenyl-5-thiazole-carboxamide; 2-chloro-N-2,3-dihydro-1,1,3-trimethyl-1H-inden-4-yl)-3-pyridinecarboxamide; 3,4,5-trichloro-2,6-pyridinedicarbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-(6-methoxy-3-pyridinyl)-cyclopropanecarboxamide; N-butyl-8-(1,1-dimethylethyl)-1-oxaspiro[4.5]decane-3-amine; sodium tetrathiocarbonate; and copper salts and preparations, such as Bordeaux mixture; copper hydroxide; copper naphthenate; copper oxychloride; copper sulphate; cufraneb; copper oxide; mancopper; oxine-copper.

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/Acaricides/Nematicides:

abamectin, ABG-9008, acephate, acequinocyl, acetamiprid, acetoprole, acrinathrin, AKD-1022, AKD-3059, AKD-3088, alanycarb, aldicarb, aldoxycarb, allethrin, allethrin 1R-isomers, alpha-cypermethrin (alphamethrin), amidoflumet, aminocarb, amitraz, avermectin, AZ-60541, azadirachtin, azamethiphos, azinphos-methyl, azinphos-ethyl, azocyclotin, Bacillus popilliae, Bacillus sphaericus, Bacillus subtilis, Bacillus thuringiensis, Bacillus thuringiensis strain EG-2348, Bacillus thuringiensis strain GC-91, Bacillus thuringiensis strain NCTC-11821, baculoviruses, Beauveria bassiana, Beauveria tenella, bendiocarb, benfuracarb, bensultap, benzoximate, beta-cyfluthrin, beta-cypermethrin, bifenazate, bifenthrin, binapacryl, bioallethrin, bioallethrin-S-cyclopentyl-isomer, bioethanomethrin, biopermethrin, bioresmethrin, bistrifluron, BPMC, brofenprox, bromophos-ethyl, bromopropylate, bromfenvinfos (-methyl), BTG-504, BTG-505, bufencarb, buprofezin, butathiofos, butocarboxim, butoxycarboxim, butyl-pyridaben, cadusafos, camphechlor, carbaryl, carbofuran, carbophenothion, carbosulphan, cartap, CGA-50439, chinomethionat, chlordane, chlordimeform, chloethocarb, chlorethoxyfos, chlorfenapyr, chlorfenvinphos, chlorfluazuron, chlormephos, chlorobenzilate, chloropicrin, chlorproxyfen, chlorpyrifosmethyl, chlorpyrifos (-ethyl), chlovaporthrin, chromafenozide, cis-cypermethrin, cis-resmethrin, cis-permethrin, clocythrin, cloethocarb, clofentezine, clothianidin, clothiazoben, codlemone, coumaphos, cyanofenphos, cyanophos, cycloprene, cycloprothrin, Cydia pomonella, cyfluthrin, cyhalothrin, cyhexatin, cypermethrin, cyphenothrin (1R-trans-isomer), cyromazine, DDT, deltamethrin, demeton-S-methyl, demeton-S-methylsulphone, diafenthiuron, dialifos, diazinon, dichlofenthion, dichlorvos, dicofol, dicrotophos, dicyclanil, diflubenzuron, dimethoate, dimethylvinphos, dinobuton, dinocap, dinotefuran, diofenolan, disulphoton, docusat-sodium, dofenapyn, DOWCO-439, eflusilanate, emamectin, emamectin-benzoate, empenthrin (1R-isomer), endosulphan, Entomopthora spp., EPN, esfenvalerate, ethiofencarb, ethiprole, ethion, ethoprophos, etofenprox, etoxazole, etrimfos, famphur, fenamiphos, fenazaquin, fenbutatin oxide, fenfluthrin, fenitrothion, fenobucarb, fenothiocarb, fenoxacrim, fenoxycarb, fenpropathrin, fenpyrad, fenpyrithrin, fenpyroximate, fensulphothion, fenthion, fentrifanil, fenvalerate, fipronil, flonicamid, fluacrypyrim, fluazuron, flubenzimine, flubrocythrinate, flucycloxuron, flucythrinate, flufenerim, flufenoxuron, flufenprox, flumethrin, flupyrazofos, flutenzin (flufenzine), fluvalinate, fonofos, formetanate, formothion, fosmethilan, fosthiazate, fubfenprox (fluproxyfen), furathiocarb, gamma-HCH, gossyplure, grandlure, granulosis viruses, halfenprox, halofenozide, HCH, HCN-801, heptenophos, hexaflumuron, hexythiazox, hydramethylnone, hydroprene, IKA-2002, imidacloprid, imiprothrin, indoxacarb, iodofenphos, iprobenfos, isazofos, isofenphos, isoprocarb, isoxathion, ivermectin, japonilure, kadethrin, nuclear polyhedrosis viruses, kinoprene, lambda-cyhalothrin, lindane, lufenuron, malathion, mecarbam, mesulphenfos, metaldehyde, metam-sodium, methacrifos, methamidophos, Metharhizium anisopliae, Metharhizium flavoviride, methidathion, methiocarb, methomyl, methoprene, methoxychlor, methoxyfenozide, metolcarb, metoxadiazone, mevinphos, milbemectin, milbemycin, MKI-245, MON45700, monocrotophos, moxidectin, MTI-800, naled, NC-104, NC-170, NC-184, NC-194, NC-196, niclosamide, nicotine, nitenpyram, nithiazine, NNI-0001, NNI-0101, NNI-0250, NNI-9768, novaluron, noviflumuron, OK-5101, OK-5201, OK-9601, OK-9602, OK-9701, OK-9802, omethoate, oxamyl, oxydemeton-methyl, Paecilomyces fumosoroseus, parathion-methyl, parathion (-ethyl), permethrin (cis-, trans-), petroleum, PH-6045, phenothrin (1R-trans isomer), phenthoate, phorate, phosalone, phosmet, phosphamidon, phosphocarb, phoxim, piperonyl butoxide, pirimicarb, pirimiphos-methyl, pirimiphos-ethyl, prallethrin, profenofos, promecarb, propaphos, propargite, propetamphos, propoxur, prothiofos, prothoate, protrifenbute, pymetrozine, pyraclofos, pyresmethin, pyrethrum, pyridaben, pyridalyl, pyridaphenthion, pyridathion, pyrimidifen, pyriproxyfen, quinalphos, resmethrin, RH-5849, ribavirin, RU-12457, RU-15525, S-421, S-1833, salithion, sebufos, SI-0009, silafluofen, spinosad, spirodiclofen, spiromesifen, sulphluramid, sulphotep, sulprofos, SZI-121, tau-fluvalinate, tebufenozide, tebufenpyrad, tebupirimfos, teflubenzuron, tefluthrin, temephos, temivinphos, terbam, terbufos, tetrachlorvinphos, tetradifon, tetramethrin, tetramethrin (1R-isomer), tetrasul, theta-cypermethrin, thiacloprid, thiamethoxam, thiapronil, thiatriphos, thiocyclam hydrogenoxalate, thiodicarb, thiofanox, thiometon, thiosultap-sodium, thuringiensin, tolfenpyrad, tralocythrn, tralomethrin, transfluthrin, triarathene, triazamate, triazophos, triazuron, trichlophenidine, trichlorfon, triflumuron, trimethacarb, vamidothion, vaniliprole, verbutin, Verticillium lecanii, WL-108477, WL-40027, YI-5201, YI-5301, YI-5302, XMC, xylylcarb, ZA-3274, zeta-cypermethrin, zolaprofos, ZXI-8901, the compound 3-methylphenyl propylcarbamate (tsumacide Z), the compound 3-(5-chloro-3-pyridinyl)-8-(2,2,2-trifluoroethyl)-8-azabicyclo[3.2.1]octane-3-carbonitrile (CAS-Reg. No. 185982-80-3) and the corresponding 3-endoisomer (CAS-Reg. No. 185984-60-5) (cf. WO-96/37494, WO-98/25923), and preparations which comprise insecticidally active plant extracts, nematodes, fungi or viruses.

A mixture with other known active compounds, such as herbicides, or with fertilizers and growth regulators, safeners and/or semiochemicals is also possible.

In addition, the compounds of the formula (I) according to the invention also have very good antimycotic activity. They have a very broad antimycotic activity spectrum in particular against dermatophytes and yeasts, moulds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The list of these fungi does by no means limit the mycotic spectrum which can be covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10,000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10,000 g/ha, preferably between 1 and 5,000 g/ha.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof, are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having new properties (“traits”) and which have been obtained by conventional breeding, by mutagenesis or by recombinant DNA techniques. They can be cultivars, varieties, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized are in particular increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits that are also particularly emphasized are the increased defence of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucoton® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned also include the varieties sold under the name Clearfield® (for example maize). Of course, these statements also apply to plant cultivars which have these genetic traits or genetic traits still to be developed, and which will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The preparation and the use of the active compounds according to the invention is illustrated by the examples below.

### PREPARATION EXAMPLES

### Example 1

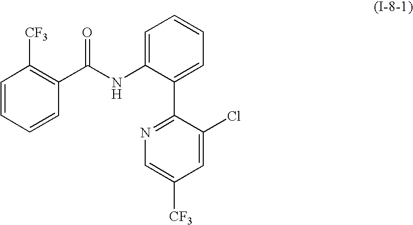

A solution of 10.0 g (25.6 mmol) N-(2-iodophenyl)-2-trifluoromethyl)benzamide, 8.0 g (31.5 mmol) bis(pinacolato)diboron, 7.4 g (75.4 mmol) potassium acetate and 0.18 g (0.25 mmol) 1,1′-bis-(diphenylphosphino)ferrocenpalladium(II)chloride in 120 ml dimethyl sulphoxide was heated under an inert gas atmosphere for 2 h at 90° C. At room temperature 6.5 g (25.0 mmol) 2-bromo-3-chloro-5(trifluoromethyl)pyridine, 70.0 ml of a 2 M sodium carbonate solution and 0.18 g (0.25 mmol) 1,1′-bis(diphenylphosphino)ferrocenepalladium(II)chloride were added. The reaction mixture was heated for 16 h at 90° C. For work-up the mixture was poured into water, extracted with ethyl acetate, dried over sodium sulphate, filtrated and concentrated in vacuo. Column chromatography (cyclohexane/ethyl acetate 3:1) yielded 1.5 g (3.4 mmol, 13%) of N-{2-[3-chloro-5-trifluoromethyl)-2-pyridinyl]phenyl}-2-(trifluoromethyl)benzamide [log P (pH 2.3)=3.58; compound No. I-8-1 in Table 3].

### Example 2

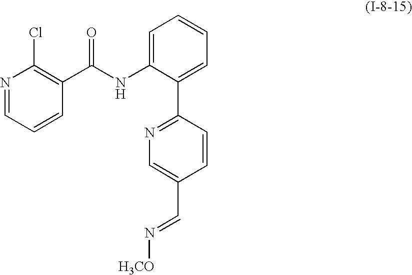

A mixture of 0.2 g (0.8 mmol) of 6-(2-amino-phenyl)-pyridine-3-carbaldehyde (E)-O-methyl-oxime (III-2), 0.155 mg (0.88 mmol) of 2-chloronicotinyl chloride, 122 mg (0.88 mmol) of potassium carbonate in 20 ml of acetonitrile was stirred for 15 hours at room temperature. For the work-up, the reaction mixture was poured into water, extracted with ethyl acetate, dried over sodium sulphate and concentrated under vacuum. The solid was triturated with petroleum ether and filtered, to give 150 mg (0.41 mmol, 41%) of 2-chloro-N-{2-[5-(E)methoxyimino-methyl)-pyridin-2-yl]-phenyl}-nicotinamide as a white powder [log P (pH 2.3)=2.95; compound No. I-8-15 in Table 3].

The pyridinylanilides of the formula (I) listed in the following tables below are likewise prepared analogously to Examples 1 and 2 described above and in accordance with the general descriptions of the processes.

TABLE 1

| | | | | | | (I-6) |
|---|---|---|---|---|---|---|
| 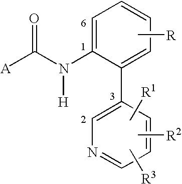 | | | | | | |
| No. | R¹ | R² | R³ | R | A | logP |
| 1-6-1 | 2-F | H | H | H | 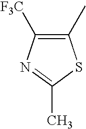 | 2.37 |
| 1-6-2 | H | H | H | 3-CH₃ | 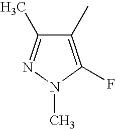 | 2.83 |
| 1-6-3 | H | H | H | H | 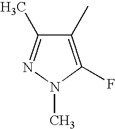 | 2.43 |
| 1-6-4 | 6-Cl | H | H | H | 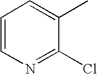 | 2.00 |
| 1-6-5 | 6-Cl | H | H | H | 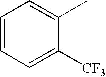 | 2.82 |
| 1-6-6 | 6-Cl | H | H | H | 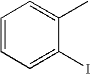 | 2.74 |
| 1-6-7 | 6-Cl | H | H | H | 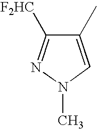 | 2.10 |
| 1-6-8 | 6-Cl | H | H | H | 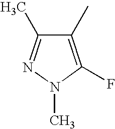 | 2.00 |
| 1-6-9 | 6-Cl | H | H | H | 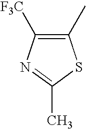 | 2.64 |

TABLE 2

| | |
|---|---|
| | (I-7) |
| 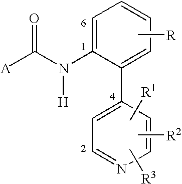 | |

| No. | R¹ | R² | R³ | R | A | logP |
|---|---|---|---|---|---|---|
| 1-7-1 | H | H | H | 4-CF₃ | 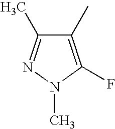 | 3.63 |
| 1-7-2 | H | H | H | H | 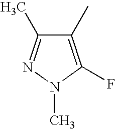 | |
| 1-7-3 | 2-Br | H | H | H | 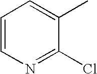 | 2.04 |
| 1-7-4 | 2-Br | H | H | H | 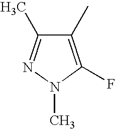 | 2.02 |
| 1-7-5 | 2-Br | H | H | H | 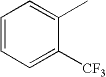 | 2.85 |
| 1-7-6 | 2-Br | H | H | H | 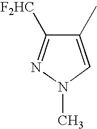 | 2.13 |
| 1-7-7 | 2-Cl | H | H | H | 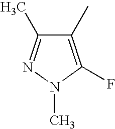 | 1.97 |
| 1-7-8 | 2-Cl | H | H | H | 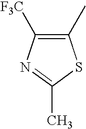 | 2.60 |
| 1-7-9 | 2-Cl | 6-Cl | H | H | 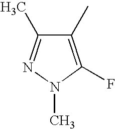 | 2.57 |
| 1-7-10 | 2-Cl | 6-Cl | H | H | 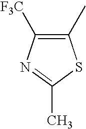 | 3.27 |

TABLE 3

| | | | | | (I-8) | |
|---|---|---|---|---|---|---|
| 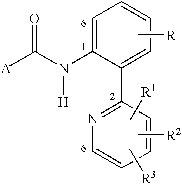 | | | | | | |
| | | | | | | logP |
| No. | R¹ | R² | R³ | R | A | m.p./° C. |
| 1-8-1 | 3-Cl | 5-CF₃ | H | H | 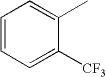 | 3.59 139 |
| 1-8-2 | 3-Cl | 5-CF₃ | H | H | 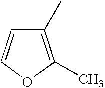 | 3.44 |
| 1-8-3 | 3-Cl | 5-CF₃ | H | H | 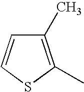 | 3.83 |
| 1-8-4 | 4-CH₃ | 5-CH═CH—CH═CH-6 | | H | 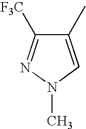 | 2.79 |
| 1-8-5 | 4-CH₃ | 5-CH═CH—CH═CH-6 | | H | 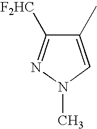 | 2.39 |
| 1-8-6 | 4-CH₃ | 5-OH═CH—CH═CH-6 | | H | 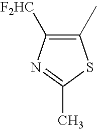 | 3.97 |
| 1-8-7 | H | H | H | 5-Cl | 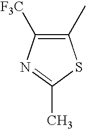 | 3.49 |
| 1-8-8 | H | H | H | 3-CH₃ | 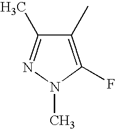 | 2.86 |
| 1-8-9 | 5-CF₃ | H | H | H | 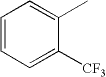 | 4.13 |
| 1-8-10 | 5-Cl | H | H | H | 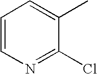 | 2.95 |
| 1-8-11 | 5-Cl | H | H | H | 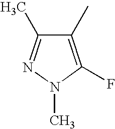 | 3.06 |
| 1-8-12 | 5-Cl | H | H | H | 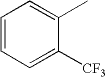 | 4.00 |
| 1-8-13 | 5-Cl | H | H | H | 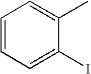 | 4.13 |
| 1-8-14 | 5-Cl | H | H | H | 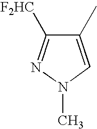 | 3.05 |
| 1-8-15 | 5-CH═N—OCH₃ | H | H | H | 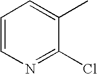 | 2.95 |
| 1-8-16 | 5-CH═N-OCH₃ | H | H | H | 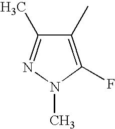 | 3.03 |
| 1-8-17 | 5-CH═N-OCH₃ | H | H | H | 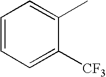 | 3.93 |
| 1-8-18 | 5-CH═N-OCH₃ | H | H | H | 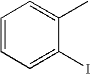 | 4.00 |
| 1-8-19 | 5-CH═N-OCH₃ | H | H | H | 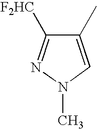 | 3.07 |
| 1-8-20 | 3-Cl | 5-CF₃ | H | H | 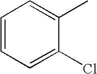 | 3.45 |
| 1-8-21 | 3-Cl | 5-CF₃ | H | H | 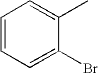 | 3.49 |
| 1-8-22 | 3-Cl | 5-CF₃ | H | H | 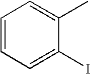 | 3.61 |
| 1-8-23 | 3-Cl | 5-CF₃ | H | H | 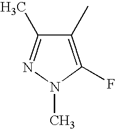 | 3.04 |
| 1-8-24 | 3-Cl | 5-CF₃ | H | H | 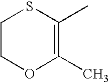 | 3.59 |
| 1-8-25 | 3-Cl | 5-CF₃ | H | H | 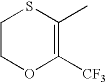 | 3.43 |
| 1-8-26 | 3-Cl | 5-CF₃ | H | H | 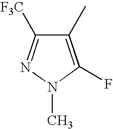 | 3.55 |
| 1-8-27 | 3-Cl | 5-CF₃ | H | H | 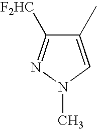 | 2.91 |
| 1-8-28 | 3-Cl | 5-CF₃ | H | H | 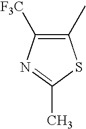 | 3.48 |
| 1-8-29 | 3-Cl | 5-CF₃ | H | H | 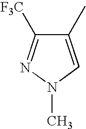 | 3.13 |
| 1-8-30 | 3-Cl | 5-CF₃ | H | H | 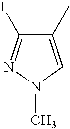 | 2.92 |
| 1-8-31 | 3-Cl | 5-CF₃ | H | H | 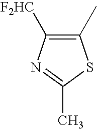 | 3.29 |
| 1-8-32 | 5-CH₃ | H | H | H | 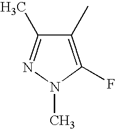 | 1.68 |
| 1-8-33 | 5-CH₃ | H | H | H | 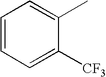 | 2.67 |
| 1-8-34 | 3-Cl | 5-Cl | H | H | 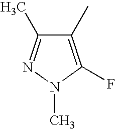 | 2.81 |
| 1-8-35 | 3-Cl | 5-Cl | H | H | 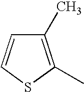 | 3.75 |
| 1-8-36 | 3-Cl | 5-Cl | H | H | 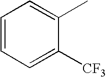 | 3.40 |
| 1-8-37 | 3-Cl | 5-Cl | H | H | 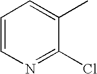 | 2.53 |
| 1-8-38 | 3-Cl | 5-Cl | H | H | 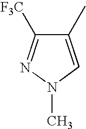 | 2.93 |
| 1-8-39 | 5-Br | H | H | H | 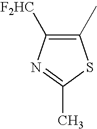 | 3.95 |
| 1-8-40 | 5-Br | H | H | H | 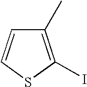 | 4.47 |
| 1-8-41 | 3-Cl | 5-Cl | H | H | 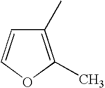 | 3.30 |
| 1-8-42 | 3-Cl | 5-Cl | H | H | 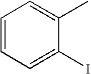 | 3.45 |
| 1-8-43 | 3-Cl | 5-Cl | H | H | 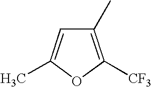 | 3.83 |
| 1-8-44 | 3-Cl | 5-Cl | H | H | 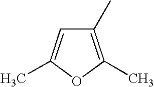 | 3.75 |
| 1-8-45 | 3-Cl | 5-Cl | H | H | 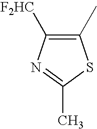 | 3.19 |
| 1-8-46 | 3-Cl | 5-Cl | H | H | 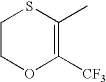 | 3.27 |
| 1-8-47 | 5-CH₃ | H | H | H | 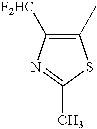 | 3.20 |
| 1-8-48 | 3-CH₃ | 5-Br | H | H | 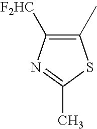 | 3.21 |
| 1-8-49 | 3-Cl | 5-Br | H | H | 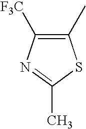 | 3.40 |
| 1-8-50 | 3-Cl | 5-Br | H | H | 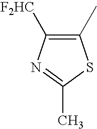 | 3.27 |
| 1-8-51 | 3-CH₃ | 5-Br | H | H | 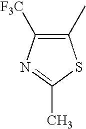 | 3.30 |
| 1-8-52 | 3-CH₃ | 5-Br | H | H | 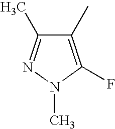 | 2.71 |
| 1-8-53 | 3-Cl | 5-Br | H | H | 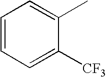 | 3.51 |
| 1-8-54 | 3-CH₃ | 5-Br | H | H | 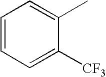 | 3.37 |
| 1-8-55 | 3-Cl | 5-Br | H | H | 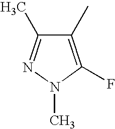 | 2.94 |

Preparation of Starting Materials of the Formula (III)

### Example (III-1)

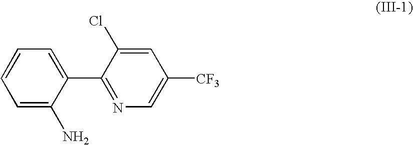

A solution of 0.67 g (3 mmol) 2-(4,4,5,5-tetramethyl-1,3,2-dioxaborolan-2-yl)aniline, 0.80 g (3 mmol) of 2-bromo-3-chloro-5-(trifluoromethyl)pyridine and 0.05 g (0.68 mmol) of 1,1′-bis(diphenylphosphino)ferrocenpalladium(II)chloride in 15 ml dimethyl sulphoxide and 9 ml of a 2 M sodium carbonate solution were heated under an inert gas atmosphere for 16 h. For the work-up the reaction mixture was poured into water, extracted with ethyl acetate, dried over sodium sulphate and concentrated in vacuo. Purification via column chromatography yielded 0.79 g (2.9 mmol, 95%) of 2-[3-chloro-5-(trifluoromethyl)-2-pyridinyl]aniline [log P (pH 2.3)=2.79].

### Example (III-2)

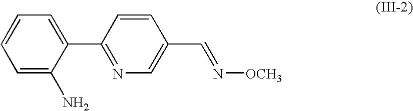

A mixture of 2.852 g (13 mmol) of 2-iodoaniline, 4.298 g of (17 mmol) of bis(pinacolato)diboron, 3.834 g (39 mmol) of potassium acetate and 24 mg (0.065 mmol) of bis(diphenylphosphino)ferrocenepalladium(II)chloride were stirred in 200 ml of dimethyl formamide at 80° C. under argon. After 4 hours, 7 g (33 mmol) of 6-bromo-pyridine-3-carbaldehyde (E)-O-methyl-oxime (VII-1), 8.28 g (78 mmol) of sodium carbonate, 100 ml of water and another 24 mg (0.065 mmol) of bis(diphenylphosphino)ferrocenpalladium(II)chloride were added and the mixture was stirred for 12 hours at 80° C. The reaction mixture was cooled down to room temperature, poured into water, extracted with ethyl acetate, dried over sodium sulphate and concentrated under vacuum. Purification via column chromatography yielded 0.9 g (4.2 mmol, 32%) of 6-(2-amino-phenyl)-pyridine-3-carbaldehyde (E)-O-methyl-oxime [log P (pH 2.3)=1.59].

Preparation of Starting Materials of the Formula (IV)

### Example (IV-1)

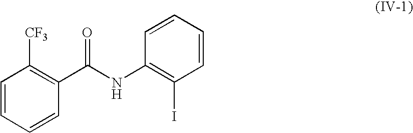

To a solution of 13.1 g (0.06 mol) o-iodoaniline and 12.1 g (0.12 mol) triethylamine in 250 ml tetrahydrofuran was added a solution of 15.0 g (0.07 mol) o-trifluoromethyl benzoic acid chloride in 250 ml tetrahydrofuran at 0° C. The reaction mixture was stirred for 30 min at 0° C. and for 16 h at room temperature. Concentration in vacuo and column chromatography (ethyl acetate) yielded 23 g (0.06 mmol, 96%) of N-2-iodophenyl)-2-(trifluoromethyl)benzamide [log P (pH 2.3)=2.98].

Preparation of Starting Materials of the Formula (VII)

### Example (VII-1)

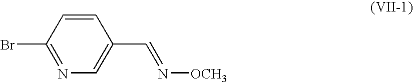

10 g of 2-bromopyridine-5-carboxaldehyde (54 mmol) and 5.84 g (70 mmol) of O-methyl-hydroxylamine hydrochloride were dissolved in 100 ml of methanol and 50 ml of water. The mixture was stirred for 20 hours at room temperature. Methanol was then evaporated under reduced pressure, water added to the solid residue that was then filtered to afford 7 g (32 mmol) of 6-bromo-pyridine-3-carbaldehyde (E)-O-methyl-oxime [log P (pH 2.3)=2.16].

The logP values given in the Preparation Examples were determined in accordance with EEC Directive 79/831 Annex V.A8 by HPLC (High Performance Liquid Chromatography) using a reversed-phase column (C 18). Temperature: 43° C.

Mobile phases for the determination in the acidic range: 0.1% aqueous phosphoric acid, acetonitrile; linear gradient from 10% acetonitrile to 90% acetonitrile.

Calibration was carried out using straight-chain alkan-2-ones (having 3 to 16 carbon atoms) with known logp values (determination of the logp values by the retention times using linear interpolation between two successive alkanones).

The lambda max values were determined in the maxima of the chromatographic signals using the UV spectra from 200 nm to 400 nm.

### Use Examples

### Example A

Podosphaera test (apple)/protective

| | |
|---|---|
| Solvent: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of the apple mildew pathogen Podosphaera leucotricha. The plants are then placed in a greenhouse at about 23° C. and a relative atmospheric humidity of about 70%.

Evaluation is carried out 10 days after the inoculation. 0% means an efficacy which corresponds to that of the control, whereas an efficacy of 100% means that no infection is observed.

Active compounds, application rates and test results are shown in the table below.

TABLE A
Podosphaera test (apple)/protective

| | Application rate | |
|---|---|---|
| Active compound | of active | Efficacy |
| according to the invention | compound in g/ha | in % |
| 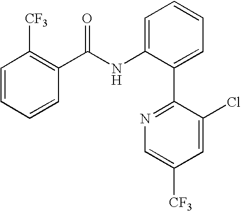 | 100 | 100 |
| (I-8-1) | | |
| 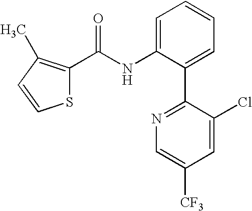 | 100 | 100 |
| (I-8-3) | | |
| 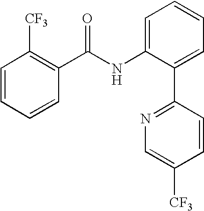 | 100 | 97 |
| (I-8-9) | | |
| 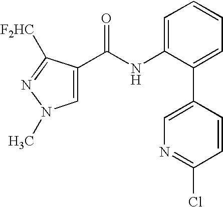 | 100 | 98 |
| (I-6-7) | | |
| 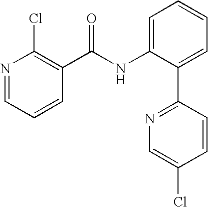 | 100 | 100 |
| (I-8-10) | | |
| 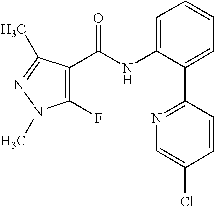 | 100 | 95 |
| (I-8-11) | | |
| 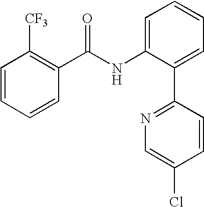 | 100 | 92 |
| (I-8-12) | | |
| 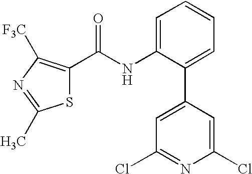 | 100 | 100 |
| (I-7-10) | | |
| 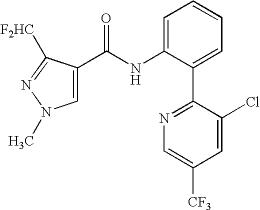 | 100 | 97 |
| (I-8-27) | | |
| 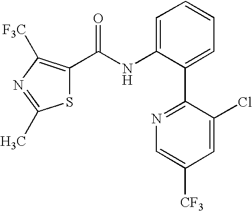 | 100 | 96 |
| (I-8-28) | | |
| 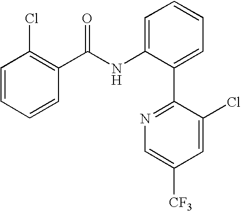 | 100 | 100 |
| (I-8-20) | | |
| 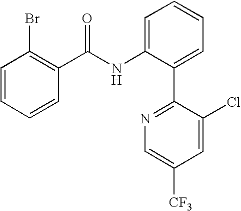 | 100 | 100 |
| (I-8-21) | | |
| 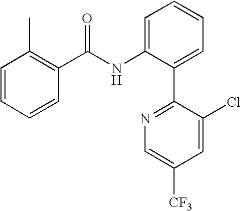 | 100 | 100 |
| (I-8-22) | | |

### Example B

Venturia Test (Apple)/protective

| | |
|---|---|
| Solvents: | 24.5 parts by weight acetone |
| | 24.5 parts by weight dimethylacetamide |
| Emulsifier: | 1.0 part by weight alkylaryl polyglycol ether |

To produce a suitable preparation of the active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, the plants are inoculated with an aqueous conidia suspension of the apple scab pathogen Venturia inaequalis and then remain in an incubation cabin at about 20° C. and 100% relative atmospheric humidity for 1 day.

The plants are then placed in a greenhouse at about 21° C. and a relative atmospheric humidity of about 90%.

Evaluation is carried out 10 days after the inoculation. 0% means an efficacy which corresponds to that of the control, whereas an efficacy of 100% means that no infection is observed.

Active compounds, application rates and test results are shown in the table below.

TABLE B
Venturia Test (Apple)/protective

| | Application rate | |
|---|---|---|
| Active compound | of active | Efficacy |
| according to the invention | compound in g/ha | in % |
| 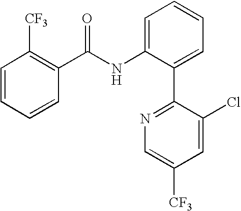 | 100 | 100 |
| (I-8-1) | | |
| 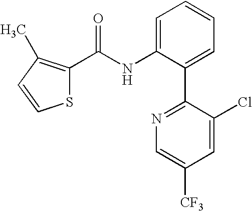 | 100 | 100 |
| (I-8-3) | | |
| 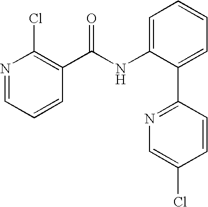 | 100 | 100 |
| (I-8-10) | | |
| 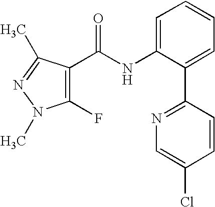 | 100 | 100 |
| (I-8-11) | | |
| 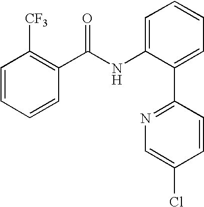 | 100 | 100 |
| (I-8-12) | | |
| 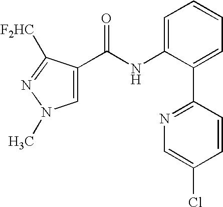 | 100 | 100 |
| (I-8-14) | | |
| 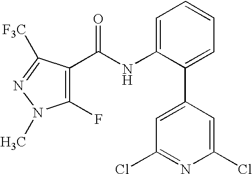 | 100 | 100 |
| (I-7-9) | | |
| 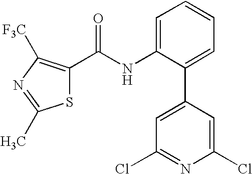 | 100 | 100 |
| (I-7-10) | | |
| 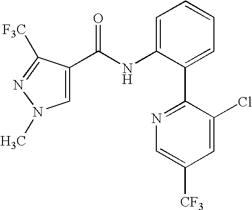 | 100 | 98 |
| (I-8-29) | | |
| 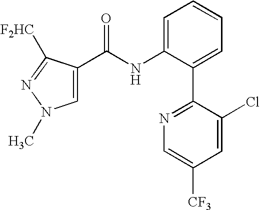 | 100 | 100 |
| (I-8-27) | | |
| 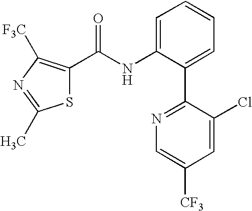 | 100 | 98 |
| (I-8-28) | | |
| 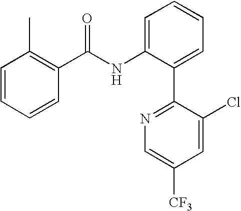 | 100 | 99 |
| (I-8-22) | | |
| 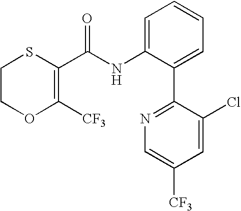 | 100 | 99 |
| (I-8-25) | | |
| 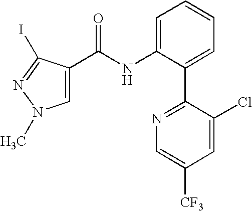 | 100 | 92 |
| (I-8-30) | | |

### Example C

Alternaria test (tomato)/protective

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young tomato plants are sprayed with the preparation of active compound at the stated application rate. 1 day after the treatment, the plants are inoculated with a spore suspension of Alternaria solani and then remain at 100% rel. humidity and 20° C. for 24 h. The plants then remain at 96% rel. atmospheric humidity and a temperature of 20° C.

Evaluation was carried out 7 days after the inoculation. 0% means an efficacy which corresponds to that of the control, whereas an efficacy of 100% means that no infection is observed.

Active compounds, application rates and test results are shown in the table below.

TABLE C
Alternaria test (tomato)/protective

| | Application rate | |
|---|---|---|
| Active compound | of active | Efficacy |
| according to the invention | compound in g/ha | in % |
| 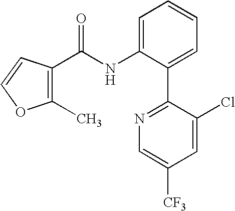 | 750 | 100 |
| (I-8-2) | | |
| 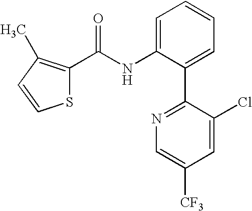 | 750 | 95 |
| (I-8-3) | | |
| 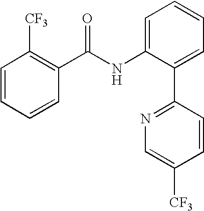 | 750 | 95 |
| (I-8-9) | | |
| 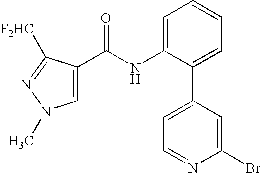 | 750 | 95 |
| (I-7-6) | | |
| 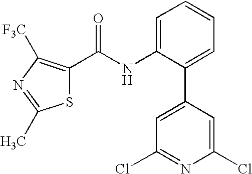 | 750 | 100 |
| (I-7-10) | | |

### Example D

Pyrenophora teres test (barley)/protective

| | |
|---|---|
| Solvent: | 50 parts by weight of N,N-dimethylacetamide |
| Emulsifier: | 1 parts by weight of alkylaryl polyglycol ether |

To produce a suitable preparation of active compound, 1 part by weight of active compound is mixed with the stated amount of solvent and emulsifier, and the concentrate is diluted with water to the desired concentration.

To test for protective activity, young plants are sprayed with the preparation of active compound at the stated application rate. After the spray coating has dried on, the plants are sprayed with a conidia suspension of Pyrenophora teres. The plants remain in an incubation cabin at 20° C. and 100% relative atmospheric humidity for 48 hours.

The plants are then placed in a greenhouse at a temperature of about 20° C. and a relative atmospheric humidity of about 80%.

Evaluation is carried out 8 days after the inoculation. 0% means an efficacy which corresponds to that of the control, whereas an efficacy of 100% means that no infection is observed.

Active compounds, application rates and test results are shown in the table below.

TABLE D
Pyrenophora teres test (barley)/protective

| | Application rate | |
|---|---|---|
| Active compound | of active | Efficacy |
| according to the invention | compound in g/ha | in % |
| 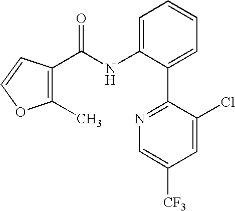 | 500 | 100 |
| (I-8-2) | | |
| 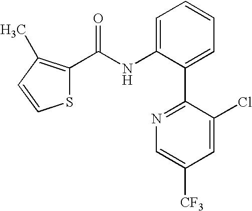 | 500 | 100 |
| (I-8-3) | | |
| 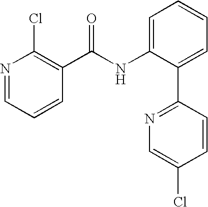 | 500 | 100 |
| (I-8-10) | | |
| 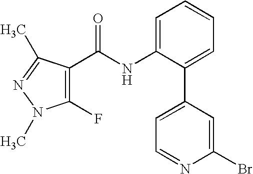 | 500 | 93 |
| (I-7-4) | | |
| 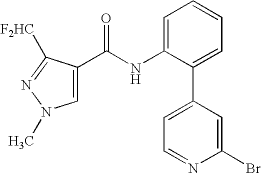 | 500 | 93 |
| (I-7-6) | | |
| 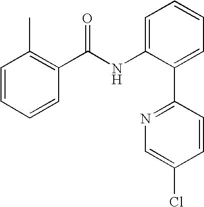 | 500 | 94 |
| (I-8-13) | | |
| 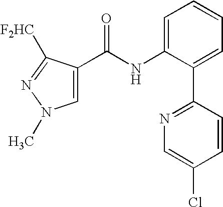 | 500 | 94 |
| (I-8-14) | | |
| 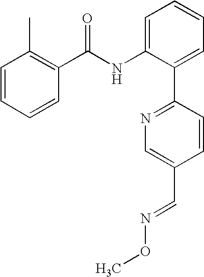 | 500 | 94 |
| (I-8-18) | | |
| 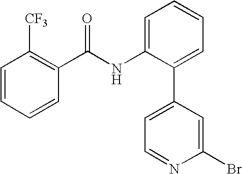 | 500 | 93 |
| (I-7-5) | | |

### Example B

Puccinia test (wheat)/protective

| | |
|---|---|
| Solvent: | 50 parts by weight of N,N-dimethylacetamide |
| Emulsifier: | >1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are sprayed with a conidia suspension of Puccinia recondita. The plants remain in an incubation cabin at 20° C. and 100% relative atmospheric humidity for 48 hours.

The plants are then placed in a greenhouse at a temperature of approximately 20° C. and a relative atmospheric humidity of 80% to promote the development of rust pustules.

Evaluation is carried out 10 days after the inoculation. 0% means an efficacy which corresponds to that of the control, whereas an efficacy of 100% means that no infection is observed.

Active compounds, application rates and test results are shown in the table below.

TABLE E
Puccinia test (wheat)/protective

| | Application rate | |
|---|---|---|
| Active compound | of active | Efficacy |
| according to the invention | compound in g/ha | in % |
| 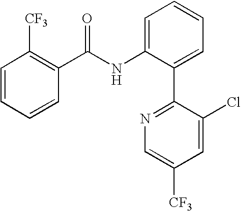 | 500 | 100 |
| (I-8-1) | | |
| 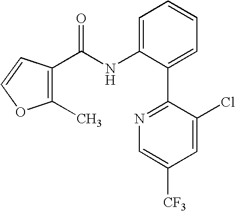 | 500 | 100 |
| (I-8-2) | | |
| 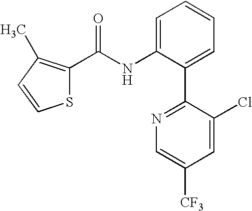 | 500 | 100 |
| (I-8-3) | | |
| 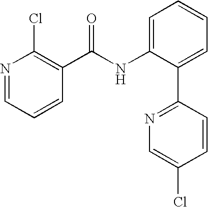 | 500 | 94 |
| (I-8-10) | | |
| 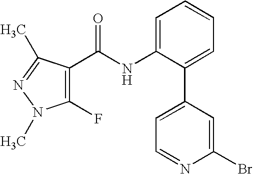 | 500 | 100 |
| (I-7-4) | | |
| 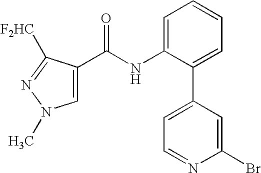 | 500 | 100 |
| (I-7-6) | | |

What is claimed is:

1-21. (canceled)

22. A pyridinylanilide of formula (I) 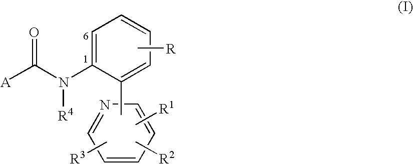
in which
R represents hydrogen, fluorine, chlorine, methyl, or trifluoromethyl;
R¹, R², and R³ independently of one another represent hydrogen, halogen, cyano, nitro, amino, hydroxyl, formyl, carboxyl, carbamoyl, or thiocarbamoyl; represent straight-chain or branched alkyl, hydroxyalkyl, oxoalkyl, alkoxy, alkoxyalkyl, alkylthioalkyl, dialkoxyalkyl, alkylthio, alkylsulfinyl, or alkylsulfonyl having in each case 1 to 8 carbon atoms in the respective alkyl moiety; represent straight-chain or branched alkenyl or alkenyloxy having in each case 2 to 6 carbon atoms; represent straight-chain or branched halogenoalkyl, halogenoalkoxy, halogenoalkylthio, halogenoalkylsulfinyl, or halogenoalkylsulfonyl having in each case 1 to 6 carbon atoms and 1 to 13 identical or different halogen atoms; represent straight-chain or branched halogenoalkenyl or halogenoalkenyloxy having in each case 2 to 6 carbon atoms and 1 to 11 identical or different halogen atoms; represent straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, or dialkylaminocarbonyloxy having 1 to 6 carbon atoms in the respective hydrocarbon chain; represent alkenylcarbonyl or alkynylcarbonyl having 2 to 6 carbon atoms in the respective hydrocarbon chain; represent cycloalkyl or cycloalkyloxy having in each case 3 to 6 carbon atoms; represent the group —C(Q¹)=N-Q², wherein
Q¹ represents hydrogen, hydroxyl, C₁-C₄-alkyl, C₁-C₄-halogenoalkyl having 1 to 9 identical or different halogen atoms, or C₃-C₆-cycloalkyl and
Q² represents hydroxyl, amino, methylamino, phenyl, or benzyl; represents C₁-C₄-alkyl or C₁-C₄-alkoxy, each of which is optionally substituted by halogen, cyano, hydroxyl, C₁-C₄-alkoxy, C₁-C₄-alkylthio, C₁-C₄-alkylamino, di(C₁-C₄-alkyl)amino, or phenyl; or represents C₂-C₄-alkenyloxy or C₂-C₄-alkynyloxy;
represents phenyl, phenoxy, phenylthio, benzoyl, benzoylethenyl, cinnamoyl, or heterocyclyl, each of which is optionally mono- to tri-substituted, identically or differently, in the ring moiety by halogen or straight-chain or branched C₁-C₄-alkyl and C₁-C₄-alkoxy; or represents phenylalkyl, phenylalkyloxy, phenylalkylthio, or heterocyclylalkyl having in each case 1 to 3 carbon atoms in the respective alkyl moieties, each of which is optionally mono- to tri-substituted, identically or differently, in the ring moiety by halogen or straight-chain or branched C₁-C₄-alkyl and C₁-C₄-alkoxy;
or when R² and R³ are attached to the pyridinyl moiety in an ortho position to each other, then R¹ is defined as above and R² and R³ together further represent C₃-C₄-alkylene, C₃-C₄-alkenylene, C₂-C₃-oxyalkylene, or C₁-C₂-dioxyalkylene, each of which is optionally mono- to tetra-substituted, identically or differently, by fluorine, chlorine, oxo, methyl, ethyl, or trifluoromethyl;
R⁴ represents hydrogen, C₁-C₈-alkyl, C₁-C₆-alkylsulfinyl, C₁-C₆-alkylsulfonyl, C₁-C₄-alkoxy-C₁-C₄-alkyl, or C₃-C₈-cycloalkyl; represents C₁-C₆-halogenoalkyl, C₁-C₄-halogenoalkylthio, C₁-C₄-halogenoalkylsulfinyl, C₁-C₄-halogenoalkylsulfonyl, halogeno-C₁-C₄-alkoxy-C₁-C₄-alkyl, or C₃-C₈-halogenocycloalkyl having in each case 1 to 9 fluorine-, chlorine-, and/or bromine atoms; represents formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C₃-halogenoalkyl)carbonyl-C₁-C₃-alkyl, or (C₁-C₃-halogenoalkoxy)carbonyl-C₁-C₃-alkyl having in each case 1 to 7 fluorine-, chlorine-, and/or bromine atoms; represents (C₁-C₃-alkyl)carbonyl-C₁-C₃-halogenoalkyl or (C₁-C₃-alkoxy)carbonyl-C₁-C₃-halogenoalkyl having in each case 1 to 6 fluorine-, chlorine- ,and/or bromine atoms represents (C₁-C₃-halogenoalkyl)carbonyl-C₁-C₃-halogenoalkyl, (C₁-C₃-halogenoalkoxy)-carbonyl-C₁-C₃-halogenoalkyl having in each case 1 to 13 fluorine-, chlorine-, and/or bromine atoms; or represents —COR⁵, —CONR⁶R⁷, or —CH₂NR⁸R⁹,
R⁵ represents hydrogen, C₁-C₈-alkyl, C₁-C₈-alkoxy, C₁-C₄-alkoxy-C₁-C₄-alkyl, or C₃-C₈-cycloalkyl; represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms; or represents —COR¹⁰,
R⁶ and R⁷ independently of one another represent hydrogen, C₁-C₈-alkyl, C₁-C₄-alkoxy-C₁-C₄-alkyl, or C₃-C₈-cycloalkyl; or represent C₁-C₈-halogenoalkyl, halogeno-C₁-C₄-alkoxy-C₁-C₄-alkyl, or C₃-C₈-halogenocycloalkyl having in each case 1 to 9 fluorine-, chlorine-, and/or bromine atoms; or R⁶ and R⁷ together with the nitrogen atom to which they are attached represent a saturated 5- to 8-membered heterocycle, wherein the heterocycle optionally has 1 or 2 additional non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R⁸ and R⁹ independently of one another represent hydrogen, C₁-C₈-alkyl, or C₃-C₈-cycloalkyl; or represent C₁-C₈-halogenoalkyl, C₃-C₈-halogenocycloalkyl having in each case 1 to 9 fluorine-, chlorine- and/or bromine atoms; or R⁸ and R⁹ together with the nitrogen atom to which they are attached, represent a saturated 5- to 8-membered heterocycle, wherein the heterocycle optionally has 1 or 2 additional, non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R¹⁰ represents hydrogen, C₁-C₈-alkyl, C₁-C₈-alkoxy, C₁-C₄-alkoxy-C₁-C₄-alkyl, or C₃-C₈-cycloalkyl; or represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms,
R¹¹ represents hydrogen or C₁-C₆-alkyl, and
A represents
(1) a radical of formula (A1) 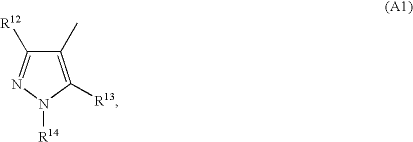
wherein
R¹² represents hydrogen, cyano, halogen, nitro, C₁-C₄-alkyl, C₁-C₄-alkoxy, C₁-C₄-alkylthio, or C₃-C₆-cycloalkyl; represents C₁-C₄-halogenoalkyl, C₁-C₄-halogenoalkoxy, or C₁-C₄-halogenoalkylthio each having 1 to 5 halogen atoms; or represents aminocarbonyl or aminocarbonyl-C₁-C₄-alkyl,
R¹³ represents hydrogen, halogen, cyano, C₁-C₄-alkyl, C₁-C₄-alkoxy, or C₁-C₄-alkylthio, and
R¹⁴ represents hydrogen, C₁-C₄-alkyl, hydroxy-C₁-C₄-alkyl, C₂-C₆-alkenyl, C₃-C₆-cycloalkyl, C₁-C₄-alkylthio-C₁-C₄-alkyl, or C₁-C₄-alkoxy-C₁-C₄-alkyl; represents C₁-C₄-halogenoalkyl, C₁-C₄-halogenoalkylthio-C₁-C₄-alkyl, or C₁-C₄-halogenoalkoxy-C₁-C₄-alkyl each having 1 to 5 halogen atoms; or represents phenyl, or
(2) a radical of formula (A2) 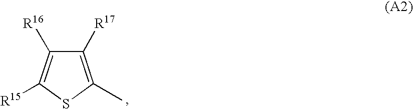
wherein
R¹⁵ and R¹⁶ independently of one another represent hydrogen, halogen, C₁-C₄-alkyl, or C₁-C₄-halogenoalkyl having 1 to 5 halogen atoms, and
R¹⁷ represents halogen, cyano or C₁-C₄-alkyl; or represents C₁-C₄-halogenoalkyl or C₁-C₄-halogenoalkoxy each having 1 to 5 halogen atoms, or
(3) a radical of formula (A3) 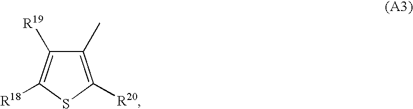
wherein
R¹⁸ and R¹⁹ independently of one another represent hydrogen, halogen, C₁-C₄-alkyl, or C₁-C₄-halogenoalkyl having 1 to 5 halogen atoms, and
R²⁰ represents hydrogen, halogen, C₁-C₄-alkyl, or C₁-C₄-halogenoalkyl having 1 to 5 halogen atoms, or
(4) a radical of formula (A4) 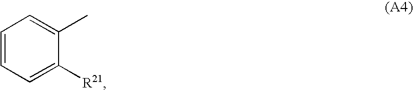
wherein R²¹ represents hydrogen, halogen, hydroxyl, cyano, or C₁-C₆-alkyl; or represents C₁-C₄-halogenoalkyl, C₁-C₄-halogenoalkoxy, or C₁-C₄-halogenoalkylthio each having 1 to 5 halogen atoms, or
(5) a radical of formula (A5) 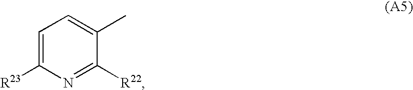
wherein
R²² represents halogen, hydroxyl, cyano, C₁-C₄-alkyl, C₁-C₄-alkoxy, or C₁-C₄-alkylthio; or represents C₁-C₄-halogenoalkyl, C₁-C₄-halogenoalkylthio, or C₁-C₄-halogenoalkoxy each having 1 to 5 halogen atoms, and
R²³ represents hydrogen, halogen, cyano, C₁-C₄-alkyl, C₁-C₄-alkoxy, or C₁-C₄-alkylthio; represents C₁-C₄-halogenoalkyl or C₁-C₄-halogenoalkoxy each having 1 to 5 halogen atoms; or represents C₁-C₄-alkylsulphinyl or C₁-C₄-alkylsulphonyl, or
(6) a radical of formula (A6) 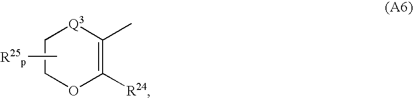
wherein
R²⁴ represents C₁-C₄-alkyl or C₁-C₄-halogenoalkyl having 1 to 5 halogen atoms,
R²⁵ represents C₁-C₄-alkyl,
Q³ represents a sulphur or oxygen atom, SO, SO₂, or CH₂, and
p represents 0, 1, or 2, with the proviso that R²⁵ represents identical or different radicals if p represents 2, or
(7) a radical of formula (A7) 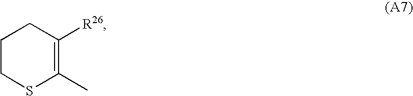
wherein R²⁶ represents C₁-C₄-alkyl or C₁-C₄-halogenoalkyl having 1 to 5 halogen atoms, or
(8) a radical of formula (A8) 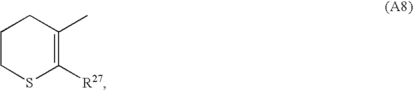
wherein R²⁷ represents C₁-C₄-alkyl or C₁-C₄-halogenoalkyl having 1 to 5 halogen atoms, or
(9) a radical of formula (A9) 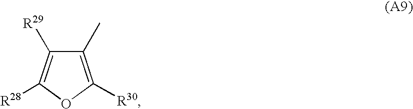
wherein
R²⁸ and R²⁹ independently of one another represent hydrogen, halogen, amino, or C₁-C₄-alkyl; or represent C₁-C₄-halogenoalkyl having 1 to 5 halogen atoms, and
R³⁰ represents hydrogen, halogen, C₁-C₄-alkyl, or C₁-C₄-halogenoalkyl having 1 to 5 halogen atoms, or
(10) a radical of formula (A10) 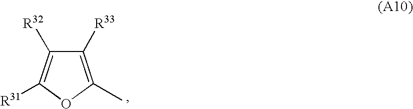
wherein
R³¹ and R³² independently of one another represent hydrogen, halogen, amino, nitro, C₁-C₄-alkyl, or C₁-C₄-halogenoalkyl having 1 to 5 halogen atoms, and
R³³ represents hydrogen, halogen, C₁-C₄-alkyl, or C₁-C₄-halogenoalkyl having 1 to 5 halogen atoms, or
(11) a radical of formula (A11) 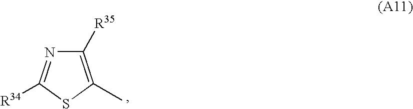
wherein
R³⁴ represents hydrogen, halogen, amino, C₁-C₄-alkylamino, di(C₁-C₄-alkyl)amino, cyano, C₁-C₄-alkyl, or C₁-C₄-halogenoalkyl having 1 to 5 halogen atoms, and
R³⁵ represents halogen, C₁-C₄-alkyl, or C₁-C₄-halogenoalkyl having 1 to 5 halogen atoms, or
(12) a radical of formula (A12) 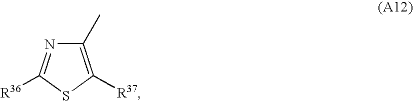
wherein
R³⁶ represents hydrogen, halogen, amino, C₁-C₄-alkylamino, di(C₁-C₄-alkyl)amino, cyano, C₁-C₄-alkyl, or C₁-C₄-halogenoalkyl having 1 to 5 halogen atoms, and
R³⁷ represents halogen, C₁-C₄-alkyl, or C₁-C₄-halogenoalkyl having 1 to 5 halogen atoms, or
(13) a radical of formula (A13) 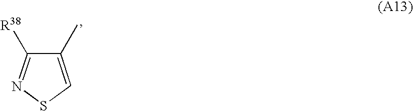
wherein R³⁸ represents halogen, C₁-C₄-alkyl, or C₁-C₄-halogenoalkyl having 1 to 5 halogen atoms, or
(14) a radical of formula (A14) 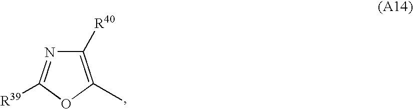
wherein
R³⁹ represents hydrogen or C₁-C₄-alkyl, and
R⁴⁰ represents halogen or C₁-C₄-alkyl, or
(15) a radical of formula (A15) 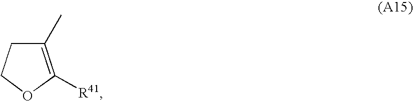
wherein R⁴¹ represents C₁-C₄-alkyl or C₁-C₄-halogenoalkyl having 1 to 5 halogen atoms, or
(16) a radical of formula (A16) 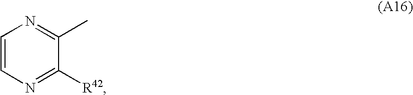
wherein R⁴² represents hydrogen, halogen, C₁-C₄-alkyl, or C₁-C₄-halogenoalkyl having 1 to 5 halogen atoms, or
(17) a radical of formula (A17) 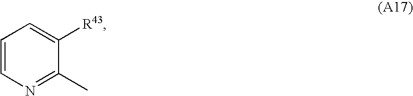
wherein R⁴³ represents halogen, hydroxyl, C₁-C₄-alkyl, C₁-C₄-alkoxy, or C₁-C₄-alkylthio, or represents C₁-C₄-halogenoalkyl, C₁-C₄-halogenoalkylthio, or C₁-C₄-halogenoalkoxy each having 1 to 5 halogen atoms, with the exception of pyridinylanilides of formula (I) in which
R represents hydrogen,
R¹, R², and R³ independently of one another each represents hydrogen; halogen; straight-chain or branched alkyl having 1 to 4 carbon atoms; or straight-chain or branched halogenoalkyl having 1 to 4 carbon atoms,
R⁴ represents hydrogen, and
A represents
(i) a radical of formula (A1) 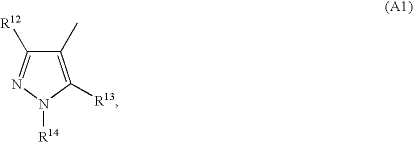
wherein
R¹² represents halogen, C₁-C₄-alkyl, or C₁-C₄-halogenoalkyl,
R¹³ represents hydrogen, and
R¹⁴ represents methyl, or
(ii) a radical of formula (A2) 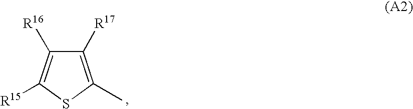
wherein
R¹⁵ and R¹⁶ independently of one another represent hydrogen or C₁-C₄-alkyl, and
R¹⁷ represents halogen, C₁-C₄-alkyl, or C₁-C₄-halogenoalkyl, or
(iii) a radical of formula (A4) 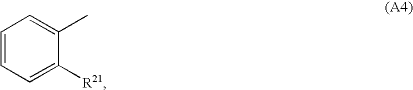
wherein R²¹ represents halogen, C₁-C₄-alkyl, or C₁-C₄-halogenoalkyl, or
(iv) a radical of formula (A5) 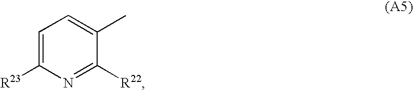
wherein
R²² represents halogen, and
R²³ represents hydrogen, or
(v) a radical of formula (A6) 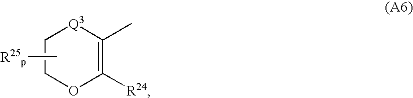
wherein
R²⁴ represents methyl,
Q³ represents a sulphur atom or CH₂, and
p represents 0, or
(vi) a radical of formula (A9) 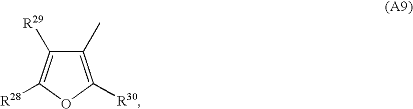
wherein
R²⁸ and R²⁹ independently of one another each represent hydrogen or C₁-C₄-alkyl, and
R³⁰ represents methyl, or
(vii) a radical of formula (A11) 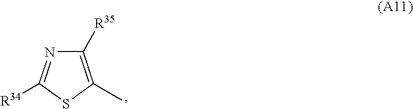
wherein
R³⁴ represents hydrogen or C₁-C₄-alkyl, and
R³⁵ represents halogen, C₁-C₄-alkyl, or C₁-C₄-halogenoalkyl, or
(viii) a radical of formula (A16) 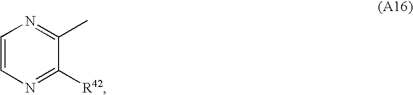
wherein R⁴² represents halogen.

23. A pyridinylanilide of formula (I) according to claim 22 in which
R represents hydrogen, fluorine, chlorine, methyl, or trifluoromethyl;
R¹, R² and R³ independently of one another represent hydrogen, halogen, cyano, nitro, amino, hydroxyl, formyl, carboxyl, carbamoyl, or thiocarbamoyl; represent straight-chain or branched alkyl, alkoxy, alkoxyalkyl, alkylthioalkyl, alkylthio, alkylsulfinyl, or alkylsulfonyl having in each case 1 to 6 carbon atoms; represent straight-chain or branched halogenoalkyl, halogenoalkoxy, halogenoalkylthio, halogenoalkylsulfinyl, or halogenoalkylsulfonyl having in each case 1 to 4 carbon atoms and 1 to 9 identical or different halogen atoms; represent straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, or dialkylaminocarbonyloxy having 1 to 4 carbon atoms in the respective hydrocarbon chain; represent cycloalkyl or cycloalkyloxy having in each case 3 to 6 carbon atoms; or represent the group —C(Q¹)=N-Q², wherein
Q¹ represents hydrogen, hydroxyl, or C₁-C₄-alkyl; represents C₁-C₄-halogenoalkyl having 1 to 9 identical or different halogen atoms; or represents C₃-C₆-cycloalkyl, and
Q² represents hydroxyl, C₁-C₄-alkyl, or C₁-C₄-alkoxy; or represents C₁-C₄-halogenoalkyl or C₁-C₄-halogenoalkoxy each having 1 to 9 identical or different halogen atoms,
or when R² and R³ are attached to the pyridinyl moiety in an ortho position to each other, then R¹ is defined as above and R² and R³ together further represent —(CH₂)₃—, —(CH₂)₄—, —CH═CH—CH═CH—, —O(CH₂)₂—, —O(CH₂)₃—, —OCH₂O—, or —O(CH₂)₂O—, each of which is optionally mono- to tetra-substituted, identically or differently, by fluorine, chlorine, oxo, methyl, ethyl, or trifluoromethyl;
R⁴ represents hydrogen, C₁-C₆-alkyl, C₁-C₄-alkylsulfinyl, C₁-C₄-alkylsulfonyl, C₁-C₃-alkoxy-C₁-C₃-alkyl, or C₃-C₆-cycloalkyl; represents C₁-C₄-halogenoalkyl, C₁-C₄-halogenoalkylthio, C₁-C₄-halogenoalkylsulfinyl, C₁-C₄-halogenoalkylsulfonyl, halogeno-C₁-C₃-alkoxy-C₁-C₃-alkyl, or C₃-C₆-halogenocycloalkyl having in each case 1 to 9 fluorine-, chlorine-, and/or bromine atoms; represents formyl-C₁-C₃-alkyl, (C₁-C₃-alkyl)carbonyl-C₁-C₃-alkyl, or (C₁-C₃-alkoxy)carbonyl-C₁-C₃-alkyl; represents (C₁-C₃-halogenoalkyl)carbonyl-C₁-C₃-alkyl or (C₁-C₃-halogenoalkoxy)carbonyl-C₁-C₃-alkyl having in each case 1 to 7 fluorine-, chlorine-, and/or bromine atoms; represents (C₁-C₃-alkyl)carbonyl-C₁-C₃-halogenoalkyl or (C₁-C₃-alkoxy)carbonyl-C₁-C₃-halogenoalkyl having in each case 1 to 6 fluorine-, chlorine-, and/or bromine atoms; represents (C₁-C₃-halogenoalkyl)carbonyl-C₁-C₃-halogenoalkyl or (C₁-C₃-halogenoalkoxy)carbonyl-C₁-C₃-halogenoalkyl having in each case 1 to 13 fluorine-, chlorine-, and/or bromine atoms; or represents —COR⁵, —CONR⁶R⁷, or —CH₂NR⁸R⁹,
R⁵ represents hydrogen, C₁-C₆-alkyl, C₁-C₄-alkoxy, C₁-C₃-alkoxy-C₁-C₃-alkyl, or C₃-C₆-cycloalkyl; represents C₁-C₄-halogenoalkyl, C₁-C₄-halogenoalkoxy, halogeno-C₁-C₃-alkoxy-C₁-C₃-alkyl, or C₃-C₆-halogenocycloalkyl having in each case 1 to 9 fluorine-, chlorine-, and/or bromine atoms; or represents —COR¹⁰,
R⁶ and R⁷ independently of one another represent hydrogen, C₁-C₆-alkyl, or C₁-C₃-alkoxy-C₁-C₃-alkyl, C₃-C₆-cycloalkyl; or represent C₁-C₄-halogenoalkyl, halogeno-C₁-C₃-alkoxy-C₁-C₃-alkyl, or C₃-C₆-halogenocycloalkyl having in each case 1 to 9 fluorine-, chlorine-, and/or bromine atoms; or R⁶ and R⁷ together with the nitrogen atom to which they are attached, represent a saturated 5- to 8-membered heterocycle, wherein the heterocycle optionally has 1 or 2 additional, non-adjacent heteroatoms selected from the group consisting of oxygen, sulphur, and NR¹¹, and wherein the heterocycle is optionally mono- to tetra-substituted, identically or differently, by halogen or C₁-C₄-alkyl,
R⁸ and R⁹ independently of one another represent hydrogen, C₁-C₆-alkyl, or C₃-C₆-cycloalkyl; or represent C₁-C₄-halogenoalkyl, C₃-C₆-halogenocycloalkyl having in each case 1 to 9 fluorine-, chlorine-, and/or bromine atoms; or R⁸ and R⁹ together with the nitrogen atom to which they are attached, represent a saturated 5- to 8-membered heterocycle, wherein the heterocycle optionally has 1 or 2 additional, non-adjacent heteroatoms selected from the group consisting of oxygen, sulphur, and NR¹¹, and wherein the heterocycle is optionally mono- to tetra-substituted, identically or differently, by halogen or C₁-C₄-alkyl,
R¹⁰ represents hydrogen, C₁-C₆-alkyl, C₁-C₄-alkoxy, C₁-C₃-alkoxy-C₁-C₃-alkyl, or C₃-C₆-cycloalkyl; or represents C₁-C₄-halogenoalkyl, C₁-C4-halogenoalkoxy, halogeno-C₁-C₃-alkoxy-C₁-C₃-alkyl, or C₃-C₆-halogenocycloalkyl having in each case 1 to 9 fluorine-, chlorine-, and/or bromine atoms,
R¹¹ represents hydrogen or C₁-C₄-alkyl, and
A represents
(1) a radical of formula (A1) 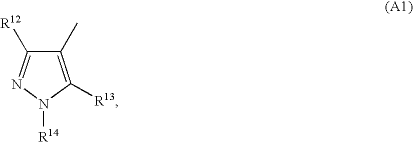
wherein
R¹² represents hydrogen, cyano, fluorine, chlorine, bromine, iodine, methyl, ethyl, iso-propyl, methoxy, ethoxy, methylthio, ethylthio, or cyclopropyl; represents C₁-C₂-halogenoalkyl or C₁-C₂-halogenoalkoxy each having 1 to 5 fluorine, chlorine, and/or bromine atoms; or represents trifluoromethylthio, difluoromethylthio, aminocarbonyl, aminocarbonylmethyl, or aminocarbonylethyl,
R¹³ represents hydrogen, fluorine, chlorine, bromine, iodine, methyl, ethyl, methoxy, ethoxy, methylthio, or ethylthio, and
R¹⁴ represents hydrogen, methyl, ethyl, n-propyl, iso-propyl, C₁-C₂-halogenoalkyl having 1 to 5 fluorine, chlorine, and/or bromine atoms, hydroxymethyl, hydroxyethyl, cyclopropyl, cyclopentyl, cyclohexyl, or phenyl, or
(2) a radical of formula (A2) 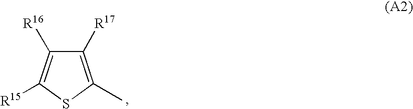
wherein
R¹⁵ and R¹⁶ independently of one another represent hydrogen, fluorine, chlorine, bromine, methyl, ethyl, or C₁-C₂-halogenoalkyl having 1 to 5 fluorine, chlorine, and/or bromine atoms and
R¹⁷ represents fluorine, chlorine, bromine, cyano, methyl, or ethyl, or represents C₁-C₂-halogenoalkyl or C₁-C₂-halogenoalkoxy each having 1 to 5 fluorine, chlorine, and/or bromine atoms, or
(3) a radical of formula (A3) 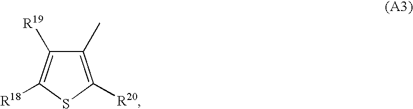
wherein
R¹⁸ and R¹⁹ independently of one another represent hydrogen, fluorine, chlorine, bromine, methyl, ethyl, or C₁-C₂-halogenoalkyl having 1 to 5 fluorine, chlorine, and/or bromine atoms, and
R²⁰ represents hydrogen, fluorine, chlorine, bromine, methyl, ethyl, or C₁-C₂-halogenoalkyl having 1 to 5 fluorine, chlorine, and/or bromine atoms, or
(4) a radical of formula (A4) 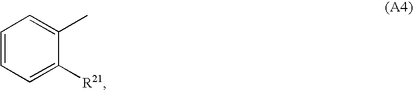
wherein R²¹ represents hydrogen, fluorine, chlorine, bromine, iodine, hydroxyl, cyano, or C₁-C₄-alkyl; or represents C₁-C₂-halogenoalkyl, C₁-C₂-halogenoalkoxy, or C₁-C₂-halogenoalkylthio each having 1 to 5 fluorine, chlorine, and/or bromine atoms, or
(5) a radical of formula (A5) 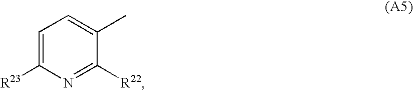
wherein
R²² represents fluorine, chlorine, bromine, iodine, hydroxyl, cyano, C₁-C₄-alkyl, methoxy, ethoxy, methylthio, ethylthio, difluoromethylthio, or trifluoromethylthio; or represents C₁-C₂-halogenoalkyl or C₁-C₂-halogenoalkoxy each having 1 to 5 fluorine, chlorine, and/or bromine atoms, and
R²³ represents hydrogen, fluorine, chlorine, bromine, iodine, cyano, C₁-C₄-alkyl, methoxy, ethoxy, methylthio, or ethylthio; represents C₁-C₂-halogenoalkyl or C₁-C₂-halogenoalkoxy each having 1 to 5 fluorine, chlorine, and/or bromine atoms; or represents C₁-C₂-alkylsulphinyl or C₁-C₂-alkylsulphonyl, or
(6) a radical of formula (A6) 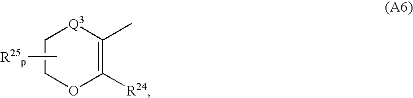
wherein
R²⁴ represents methyl, ethyl, or C₁-C₂-halogenoalkyl having 1 to 5 fluorine, chlorine and/or bromine atoms,
R²⁵ represents methyl or ethyl,
Q³ represents a sulphur atom, SO₂, or CH₂, and
p represents 0 or 1, or
(7) a radical of formula (A9) 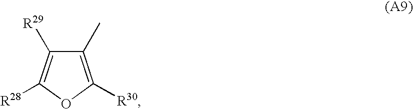
wherein
R²⁸ and R²⁹ independently of one another represent hydrogen, fluorine, chlorine, bromine, amino, methyl, ethyl, or C₁-C₂-halogenoalkyl having 1 to 5 fluorine, chlorine, and/or bromine atoms, and
R³⁰ represents hydrogen, fluorine, chlorine, bromine, iodine, methyl, ethyl, or C₁-C₂-halogenoalkyl having 1 to 5 fluorine, chlorine, and/or bromine atoms, or
(8) a radical of formula (A10) 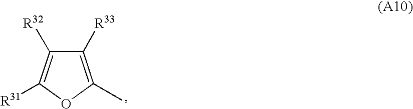
wherein
R³¹ and R³² independently of one another represent hydrogen, fluorine, chlorine, bromine, amino, nitro, methyl, ethyl, or C₁-C₂-halogenoalkyl having 1 to 5 fluorine, chlorine, and/or bromine atoms, and
R³³ represents hydrogen, fluorine, chlorine, bromine, methyl, ethyl, or C₁-C₂-halogenoalkyl having 1 to 5 fluorine, chlorine, and/or bromine atoms, or
(9) a radical of formula (A11) 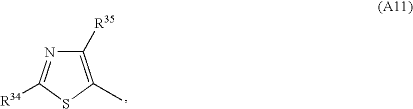
wherein
R³⁴ represents hydrogen, fluorine, chlorine, bromine, amino, C₁-C₄-alkylamino, di(C₁-C₄-alkyl)amino, cyano, methyl, ethyl, or C₁-C₂-halogenoalkyl having 1 to 5 fluorine, chlorine, and/or bromine atoms, and
R³⁵ represents fluorine, chlorine, bromine, methyl, ethyl, or C₁-C₂-halogenoalkyl having 1 to 5 fluorine, chlorine, and/or bromine atoms, or
(10) a radical of formula (A12) 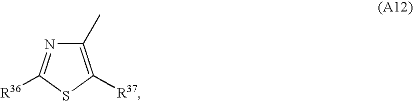
wherein
R³⁶ represents hydrogen, fluorine, chlorine, bromine, amino, C₁-C₄-alkylamino, di(C₁-C₄-alkyl)amino, cyano, methyl, ethyl, or C₁-C₂-halogenoalkyl having 1 to 5 fluorine, chlorine, and/or bromine atoms, and
R³⁷ represents fluorine, chlorine, bromine, methyl, ethyl, or C₁-C₂-halogenoalkyl having 1 to 5 fluorine, chlorine, and/or bromine atoms, or
(11) a radical of formula (A17) 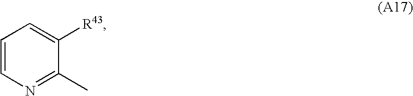
wherein R⁴³ represents fluorine, chlorine, bromine, iodine, hydroxyl, C₁-C₄-alkyl, methoxy, ethoxy, methylthio, ethylthio, difluoromethylthio, or trifluoromethylthio; or represents C₁-C₂-halogenoalkyl or C₁-C₂-halogenoalkoxy each having 1 to 5 fluorine, chlorine, and/or bromine atoms,
with the exception of pyridinylanilides of formula (I) in which
R represents hydrogen,
R¹, R², and R³ independently of one another represent hydrogen or halogen; represent straight-chain or branched alkyl having 1 to 4 carbon atoms; or represent straight-chain or branched halogenoalkyl having 1 to 4 carbon atoms,
R⁴ represents hydrogen, and
A represents
(i) a radical of formula (A1) 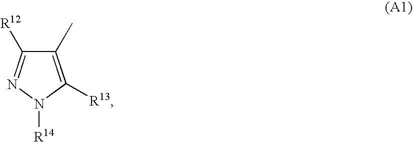
wherein
R¹² represents fluorine, chlorine, bromine, iodine, methyl, ethyl, iso-propyl, or C₁-C₂-halogenoalkyl,
R¹³ represents hydrogen, and
R¹⁴ represents methyl, or
(ii) a radical of formula (A2) 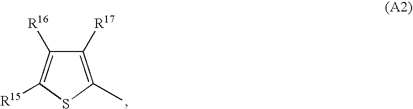
wherein
R¹⁵ and R¹⁶ independently of one another each represent hydrogen, methyl, or ethyl, and
R¹⁷ represents fluorine, chlorine, bromine, methyl, ethyl, or C₁-C₂-halogenoalkyl, or
(iii) a radical of formula (A4) 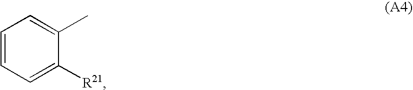
wherein R²¹ represents fluorine, chlorine, bromine, iodine, C₁-C₄-alkyl, or C₁-C₂-halogenoalkyl, or
(iv) a radical of formula (A5) 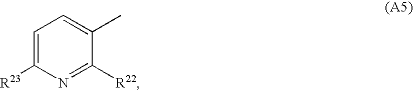
wherein
R²² represents fluorine, chlorine, bromine, or iodine, and
R²³ represents hydrogen, or
(v) a radical of formula (A6) 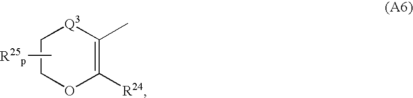
wherein
R²⁴ represents methyl,
Q³ represents a sulphur atom or CH₂, and
p represents 0, or
(vi) a radical of formula (A9) 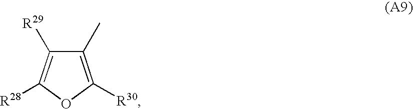
wherein
R²⁸ and R²⁹ independently of one another represent hydrogen, methyl. or ethyl, and
R³⁰ represents methyl, or
(vii) a radical of formula (A11) 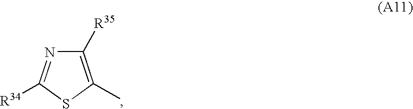
wherein
R³⁴ represents hydrogen, methyl, or ethyl, and
R³⁵ represents fluorine, chlorine, bromine, methyl, ethyl, or C₁-C₂-halogenoalkyl.

24. A pyridinylanilide of formula (I) according to claim 22 in which
R represents hydrogen, fluorine, chlorine, methyl, or trifluoromethyl; R¹, R², and R³ independently of one another represent hydrogen, fluorine, chlorine, bromine, cyano; methyl, ethyl, n- or iso-propyl, n-, iso-, sec-, or tert-butyl, methoxy, ethoxy, n- or iso-propoxy, n-, iso-, sec-, or tert-butoxy, methylthio, ethylthio, n- or iso-propylthio, n-, iso-, sec-, or tert-butylthio, trifluoromethyl, trifluoroethyl, difluoromethoxy, trifluoromethoxy, difluorochloromethoxy, trifluoroethoxy, cyclopropyl, cyclopentyl, or cyclohexyl, or represent the group —C(Q¹)=N-Q², wherein
Q¹ represents hydrogen, methyl, ethyl, trifluoromethyl, or cyclopropyl, and
Q² represents hydroxyl, methoxy, ethoxy, n-propoxy, or iso-propoxy,
or when R² and R³ are attached to the pyridinyl moiety in an ortho position to each other, then R¹ is defined as above and R² and R³ together further represent —(CH₂)₃—, —(CH₂)₄—, —CH═CH—CH═CH—, —OCH₂O—, —O(CH₂)₂O—, —OCF₂O—, or —O(CF₂)₂O—,
R⁴ represents hydrogen, methyl, ethyl, n- or iso-propyl, n-, iso-, sec-, or tert-butyl, pentyl, hexyl, methylsulfinyl, ethylsulfinyl, n- or iso-propylsulfinyl, n-, iso-, sec-, or tert-butylsulfinyl, methylsulfonyl, ethylsulfonyl, n- or iso-propylsulfonyl, n-, iso-, sec-, or tert-butylsulfonyl, methoxymethyl, methoxyethyl, ethoxymethyl, ethoxyethyl, cyclopropyl, cyclopentyl, cyclohexyl, trifluoromethyl, trichloromethyl, trifluoroethyl, difluoromethylthio, difluorochloromethylthio, trifluoromethylthio, trifluoromethylsulfinyl, trifluoromethylsulfonyl, trifluoromethoxymethyl, —CH₂—CHO, —CH₂CH₂—CHO, —CH₂—CO—CH₃, —CH₂—CO—CH₂CH₃, —CH₂—CO—CH(CH₃)₂, —CH₂CH₂—CO—CH₃, —CH₂CH₂—CO—CH₂CH₃, —CH₂CH₂—CO—CH(CH₃)₂, —CH₂—C(O)OCH₃, —CH₂—C(O)OCH₂CH₃, —CH₂—C(O)OCH(CH₃)₂, —CH₂CH₂—C(O)OCH₃, —CH₂CH₂—C(O)OCH₂CH₃, —CH₂CH₂—C(O)OCH(CH₃)₂, —CH₂—CO—CF₃, —CH₂—CO—CCl₃, —CH₂—CO—CH₂CF₃, —CH₂—CO—CH₂CCl₃, —CH₂CH₂—CO—CH₂CF₃, —CH₂CH₂—CO—CH₂CCl₃, —CH₂—C(O)OCH₂CF₃, —CH₂—C(O)OCF₂CF₃, —CH₂—C(O)OCH₂CCl₃, —CH₂—C(O)OCCl₂CCl₃, —CH₂CH₂—C(O)OCH₂CF₃, —CH₂CH₂—C(O)OCF₂CF₃, —CH₂CH₂—C(O)OCH₂CCl₃, —CH₂CH₂—C(O)O—CCl₂CCl₃; —COR⁵, —CONR⁶R⁷, or —CH₂NR⁸R⁹,
R⁵ represents hydrogen, methyl, ethyl, n- or iso-propyl, tert-butyl, methoxy, ethoxy, tert-butoxy, cyclopropyl, trifluoromethyl, trifluoromethoxy, or —COR¹⁰,
R⁶ and R⁷ independently of one another represent hydrogen, methyl, ethyl, n- or iso-propyl, n-, iso-, sec-, or tert-butyl, methoxymethyl, methoxyethyl, ethoxymethyl, ethoxyethyl, cyclopropyl, cyclopentyl, cyclohexyl; trifluoromethyl, trichloromethyl, trifluoroethyl, or trifluoromethoxymethyl; or R⁶ and R⁷ together with the nitrogen atom to which they are attached, represent a saturated heterocycle selected from the group consisting of morpholine, thiomorpholine, and piperazine, wherein the heterocycle is optionally mono- to tetra-substituted, identically or differently, by fluorine, chlorine, bromine, or methyl, and wherein the piperazine additionally at the second nitrogen atom is optionally substituted by R¹¹,
R⁸ and R⁹ independently of one another represent hydrogen, methyl, ethyl, n- or iso-propyl, n-, iso-, sec- or tert-butyl, methoxymethyl, methoxyethyl, ethoxymethyl, ethoxyethyl, cyclopropyl, cyclopentyl, cyclohexyl; trifluoromethyl, trichloromethyl, trifluoroethyl, or trifluoromethoxymethyl; or R⁸ and R⁹ together with the nitrogen atom to which they are attached, represent a saturated heterocycle selected from the group consisting of morpholine, thiomorpholine, and piperazine, wherein the heterocycle is optionally mono- to tetra-substituted, identically or differently, by fluorine, chlorine, bromine, or methyl and wherein the piperazine additionally at the second nitrogen atom is optionally substituted by R¹¹,
R¹⁰ represents hydrogen, methyl, ethyl, n- or iso-propyl, tert-butyl, methoxy, ethoxy, n- or iso-propoxy, tert-butoxy, cyclopropyl; trifluoromethyl, or trifluoromethoxy,
R¹¹ represents hydrogen, methyl, ethyl, n- or iso-propyl, or n-, iso-, sec-, or tert-butyl,
A represents
(1) a radical of formula (A1) 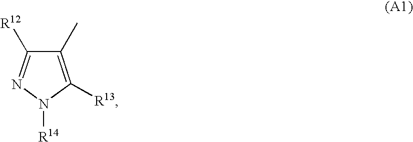
wherein
R¹² represents hydrogen, fluorine, chlorine, bromine, iodine, methyl, ethyl, iso-propyl, monofluoromethyl, monofluoroethyl, difluoromethyl, trifluoromethyl, difluorochloromethyl, trichloromethyl, dichloromethyl, cyclopropyl, methoxy, ethoxy, trifluoromethoxy, trichloromethoxy, methylthio, ethylthio, trifluoromethylthio, or difluoromethylthio,
R¹³ represents hydrogen, fluorine, chlorine, bromine, iodine, or methyl, and
R¹⁴ represents hydrogen, methyl, ethyl, iso-propyl, trifluoromethyl, difluoromethyl, hydroxymethyl, hydroxyethyl, or phenyl, or
(2) a radical of formula (A2) 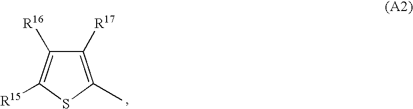
wherein
R¹⁵ and R¹5 independently of one another represent hydrogen, fluorine, chlorine, bromine, methyl, ethyl, difluoromethyl, trifluoromethyl, difluorochloromethyl, or trichloromethyl, and
R¹⁷ represents fluorine, chlorine, bromine, cyano, methyl, trifluoromethyl, trifluoromethoxy, difluoromethoxy, difluorochloromethoxy, or trichloromethoxy, or
(3) a radical of formula (A4) 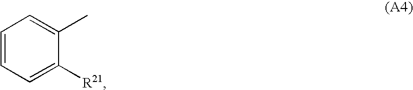
wherein R²¹ represents hydrogen, fluorine, chlorine, bromine, iodine, hydroxyl, cyano, methyl, ethyl, n-propyl, iso-propyl, n-butyl, iso-butyl, sec-butyl, tert-butyl, difluoromethyl, trifluoromethyl, difluorochloromethyl, trichloromethyl, trifluoromethoxy, difluoromethoxy, difluorochloromethoxy, trichloromethoxy, trifluoromethylthio, difluoromethylthio, difluorochloromethylthio, or trichloromethylthio, or
(4) a radical of formula (A5) 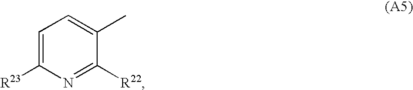
wherein
R²² represents fluorine, chlorine, bromine, iodine, hydroxyl, cyano, methyl, ethyl, n-propyl, iso-propyl, n-butyl, iso-butyl, sec-butyl, tert-butyl, trifluoromethyl, difluoromethyl, difluorochloromethyl, trichloromethyl, methoxy, ethoxy, methylthio, ethylthio, difluoromethylthio, trifluoromethylthio. trifluoromethoxy, difluoromethoxy, difluorochloromethoxy, or trichloromethoxy, and
R²³ represents hydrogen, fluorine, chlorine, bromine, iodine, cyano, n-propyl, iso-propyl, n-butyl, iso-butyl, sec-butyl, tert-butyl, trifluoromethyl, difluoromethyl, difluorochloromethyl, trichloromethyl, methoxy, ethoxy, methylthio, ethylthio, trifluoromethoxy, difluoromethoxy, difluorochloromethoxy, trichloromethoxy, methylsulphinyl, or methylsulphonyl, or
(5) a radical of formula (A6) 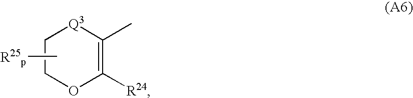
wherein
R²⁴ represents methyl, ethyl, trifluoromethyl, difluoromethyl, difluorochloromethyl, or trichloromethyl,
R²⁵ represents methyl,
Q³ represents a sulphur atom or CH₂, and
p represents 0, or
(6) a radical of formula (A9) 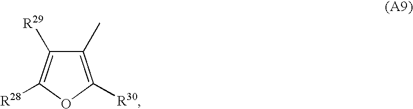
wherein
R²⁸ and R²⁹ independently of one another represent hydrogen, fluorine, chlorine, bromine, methyl, ethyl, trifluoromethyl, difluoromethyl, difluorochloromethyl, or trichloromethyl, and
R³⁰ represents hydrogen, fluorine, chlorine, bromine, iodine, methyl, ethyl, trifluoromethyl, difluoromethyl, difluorochloromethyl, or trichloromethyl, or
(7) a radical of formula (A11) 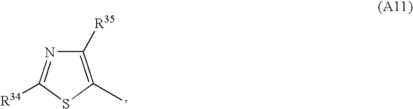
wherein
R³⁴ represents hydrogen, fluorine, chlorine, bromine, amino, methylamino, dimethylamino, cyano, methyl, ethyl, trifluoromethyl, difluoromethyl, difluorochloromethyl, or trichloromethyl, and
R³⁵ represents fluorine, chlorine, bromine, methyl, ethyl, trifluoromethyl, difluoromethyl, difluorochloromethyl, or trichloromethyl, or
(8) a radical of formula (A17) 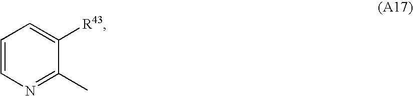
wherein R⁴³ represents fluorine, chlorine, bromine, iodine, methyl, ethyl, n-propyl, iso-propyl, n-butyl, iso-butyl, sec-butyl, tert-butyl, trifluoromethyl, difluoromethyl, difluorochloromethyl, or trichloromethyl,
with the exception of pyridinylanilides of formula (I) in which
R represents hydrogen,
R¹, R², and R³ independently of one another represent hydrogen, fluorine, chlorine, bromine; methyl, ethyl, n- or iso-propyl, n-, iso-, sec-, or tert-butyl, trifluoromethyl, or trifluoroethyl;
R⁴ represents hydrogen, and
A represents
(i) a radical of formula (A1) 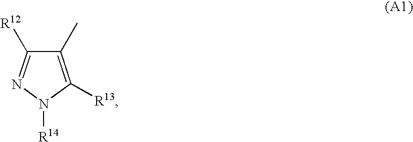
wherein
R¹² represents fluorine, chlorine, bromine, iodine, methyl, ethyl, iso-propyl, monofluoromethyl, monofluoroethyl, difluoromethyl, trifluoromethyl, difluorochloromethyl, trichloromethyl, or dichloromethyl,
R¹³ represents hydrogen, and
R¹⁴ represents methyl, or
(ii) a radical of formula (A2) 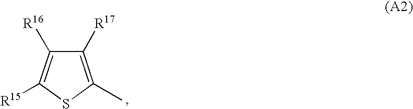
wherein
R¹⁵ and R¹⁶ independently of one another represent hydrogen, methyl, or ethyl, and
R¹⁷ represents fluorine, chlorine, bromine, methyl, ethyl, or trifluoromethyl, or
(iii) a radical of formula (A4) 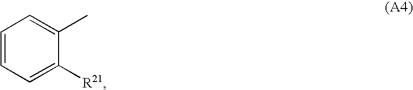
wherein R²¹ represents fluorine, chlorine, bromine, iodine, methyl, ethyl, n-propyl, iso-propyl, n-butyl, iso-butyl, sec-butyl, tert-butyl, difluoromethyl, trifluoromethyl, difluorochloromethyl, or trichloromethyl, or
(iv) a radical of formula (A5) 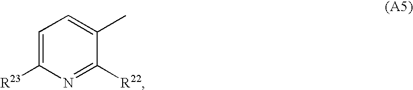
wherein
R²² represents fluorine, chlorine, bromine, or iodine, and
R²³ represents hydrogen, or
(v) a radical of formula (A6) 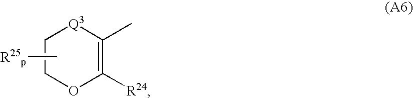
wherein
R²⁴ represents methyl,
Q³ represents a sulphur atom or CH₂, and
p represents 0, or
(vi) a radical of formula (A9) 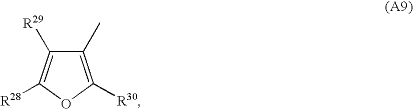
wherein
R²⁸ and R²⁹ independently of one another represent hydrogen, methyl, or ethyl, and
R³⁰ represents methyl, or
(vii) a radical of formula (A11) 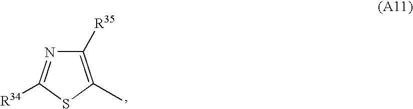
wherein
R³⁴ represents hydrogen, methyl, or ethyl, and
R³⁵ represents fluorine, chlorine, bromine, methyl, ethyl, trifluoromethyl, difluoromethyl, difluorochloromethyl, or trichloromethyl.

25. A pyridinylanilide of formula (I) according to claim 22 in which R⁴ represents hydrogen.

26. A pyridinylanilide of formula (I) according to claim 22 in which R represents hydrogen.

27. A pyridinylanilide of formula (I-12) 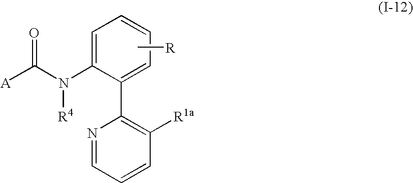
in which
R represents hydrogen, fluorine, chlorine, methyl, or trifluoromethyl;
R^{1a} represents halogen, cyano, nitro, amino, hydroxyl, formyl, carboxyl, carbamoyl, or thiocarbamoyl; represents straight-chain or branched alkyl, hydroxyalkyl, oxoalkyl, alkoxy, alkoxyalkyl, alkylthioalkyl, dialkoxyalkyl, alkylthio, alkylsulfinyl, or alkylsulfonyl having in each case 1 to 8 carbon atoms in the respective alkyl moiety; represents straight-chain or branched alkenyl or alkenyloxy having in each case 2 to 6 carbon atoms; represents straight-chain or branched halogenoalkyl, halogenoalkoxy, halogenoalkylthio, halogenoalkylsulfinyl, or halogenoalkylsulfonyl having in each case 1 to 6 carbon atoms and 1 to 13 identical or different halogen atoms; represents straight-chain or branched halogenoalkenyl or halogenoalkenyloxy having in each case 2 to 6 carbon atoms and 1 to 11 identical or different halogen atoms; represents straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, or dialkylaminocarbonyloxy having 1 to 6 carbon atoms in the respective hydrocarbon chain; represents alkenylcarbonyl or alkynylcarbonyl having 2 to 6 carbon atoms in the respective hydrocarbon chain; represents cycloalkyl or cycloalkyloxy having in each case 3 to 6 carbon atoms; represents the group —C(Q¹)=N-Q², wherein
Q¹ represents hydrogen, hydroxyl, or C₁-C₄-alkyl; represents C₁-C₄-halogenoalkyl having 1 to 9 identical or different halogen atoms; or represents C₃-C₆-cycloalkyl, and
Q² represents hydroxyl, amino, methylamino, phenyl, or benzyl; represents C₁-C₄-alkyl or C₁-C₄-alkoxy, each of which is optionally substituted by halogen, cyano, hydroxyl, C₁-C₄-alkoxy, C₁-C₄-alkylthio, C₁-C₄-alkylamino, di(C₁-C₄-alkyl)amino, or phenyl; or represents C₂-C₄-alkenyloxy or C₂-C₄-alkynyloxy;
represents phenyl, phenoxy, phenylthio, benzoyl, benzoylethenyl, cinnamoyl, or heterocyclyl; or represents phenylalkyl, phenylalkyloxy, phenylalkylthio, or heterocyclylalkyl having in each case 1 to 3 carbon atoms in the respective alkyl moieties, each of which is optionally mono- to tri-substituted, identically or differently, in the ring moiety by halogen or straight-chain or branched C₁-C₄-alkyl or C₁-C₄-alkoxy;
R⁴ represents hydrogen, C₁-C₈-alkyl, C₁-C₆-alkylsulfinyl, C₁-C₆-alkylsulfonyl, C₁-C₄-alkoxy-C₁-C₄-alkyl, or C₃-C₈-cycloalkyl; represents C₁-C₆-halogenoalkyl, C₁-C₄-halogenoalkylthio, C₁-C₄-halogenoalkylsulfinyl, C₁-C₄-halogenoalkylsulfonyl, halogeno-C₁-C₄-alkoxy-C₁ -C₄-alkyl, or C₃-C₈-halogenocycloalkyl having in each case 1 to 9 fluorine-, chlorine-, and/or bromine atoms; represents formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C₃-halogenoalkyl)carbonyl-C₁-C₃-alkyl, or (C₁-C₃-halogenoalkoxy)carbonyl-C₁-C₃-alkyl having in each case 1 to 7 fluorine-, chlorine-, and/or bromine atoms; represents (C₁-C₃-alkyl)carbonyl-C₁-C₃-halogenoalkyl or (C₁-C₃-alkoxy)carbonyl-C₁-C₃-halogenoalkyl having in each case 1 to 6 fluorine-, chlorine- ,and/or bromine atoms represents (C₁-C₃-halogenoalkyl)carbonyl-C₁-C₃-halogenoalkyl, (C₁-C₃-halogenoalkoxy)-carbonyl-C₁-C₃-halogenoalkyl having in each case 1 to 13 fluorine-, chlorine-, and/or bromine atoms; or represents —COR⁵, —CONR⁶R⁷, or —CH₂NR⁸R⁹,
R⁵ represents hydrogen, C₁-C₈-alkyl, C₁-C₈-alkoxy, C₁-C₄-alkoxy-C₁-C₄-alkyl, or C₃-C₈-cycloalkyl; represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms; or represents —COR¹⁰,
R⁶ and R⁷ independently of one another represent hydrogen, C₁-C₈-alkyl, C₁-C₄-alkoxy-C₁-C₄-alkyl, or C₃-C₈-cycloalkyl; or represent C₁-C₈-halogenoalkyl, halogeno-C₁-C₄-alkoxy-C₁-C₄-alkyl, or C₃-C₈-halogenocycloalkyl having in each case 1 to 9 fluorine-, chlorine-, and/or bromine atoms; or R⁶ and R⁷ together with the nitrogen atom to which they are attached represent a saturated 5- to 8-membered heterocycle, wherein the heterocycle optionally has 1 or 2 additional non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R⁸ and R⁹ independently of one another represent hydrogen, C₁-C₈-alkyl, or C₃-C₈-cycloalkyl; or represent C₁-C₈-halogenoalkyl, C₃-C₈-halogenocycloalkyl having in each case 1 to 9 fluorine-, chlorine- and/or bromine atoms; or R⁸ and R⁹ together with the nitrogen atom to which they are attached, represent a saturated 5- to 8-membered heterocycle, wherein the heterocycle optionally has 1 or 2 additional, non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R¹⁰ represents hydrogen, C₁-C₈-alkyl, C₁-C₈-alkoxy, C₁-C₄-alkoxy-C₁-C₄-alkyl, or C₃-C₈-cycloalkyl; or represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms,
R¹¹ represents hydrogen or C₁-C₆-alkyl, and
A represents
(1) a radical of formula (A1) 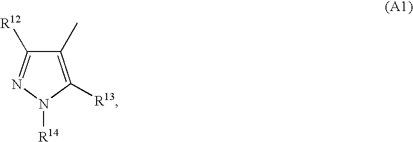
wherein
R¹² represents hydrogen, cyano, halogen, nitro, C₁-C₄-alkyl, C₁-C₄-alkoxy, C₁-C₄-alkylthio, or C₃-C₆-cycloalkyl; represents C₁-C₄-halogenoalkyl, C₁-C₄-halogenoalkoxy, or C₁-C₄-halogenoalkylthio each having 1 to 5 halogen atoms; or represents aminocarbonyl or aminocarbonyl-C₁-C₄-alkyl,
R¹³ represents hydrogen, halogen, cyano, C₁-C₄-alkyl, C₁-C₄-alkoxy, or C₁-C₄-alkylthio, and
R¹⁴ represents hydrogen, C₁-C₄-alkyl, hydroxy-C₁-C₄-alkyl, C₂-C₆-alkenyl, C₃-C₆-cycloalkyl, C₁-C₄-alkylthio-C₁-C₄-alkyl, or C₁-C₄-alkoxy-C₁-C₄-alkyl; represents C₁-C₄-halogenoalkyl, C₁-C₄-halogenoalkylthio-C₁-C₄-alkyl, or C₁-C₄-halogenoalkoxy-C₁-C₄-alkyl each having 1 to 5 halogen atoms; or represents phenyl, or
(2) a radical of formula (A2) 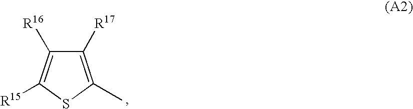
wherein
R¹⁵ and R¹⁶ independently of one another represent hydrogen, halogen, C₁-C₄-alkyl, or C₁-C₄-halogenoalkyl having 1 to 5 halogen atoms, and
R¹⁷ represents halogen, cyano or C₁-C₄-alkyl; or represents C₁-C₄-halogenoalkyl or C₁-C₄-halogenoalkoxy each having 1 to 5 halogen atoms, or
(3) a radical of formula (A3) 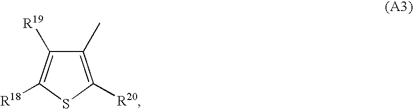
wherein
R¹⁸ and R¹⁹ independently of one another represent hydrogen, halogen, C₁-C₄-alkyl, or C₁-C₄-halogenoalkyl having 1 to 5 halogen atoms, and
R²⁰ represents hydrogen, halogen, C₁-C₄-alkyl, or C₁-C₄-halogenoalkyl having 1 to 5 halogen atoms, or
(4) a radical of formula (A4) 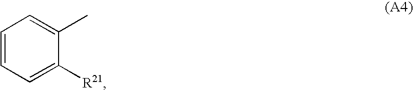
wherein R²¹ represents hydrogen, halogen, hydroxyl, cyano, or C₁-C₆-alkyl; or represents C₁-C₄-halogenoalkyl, C₁-C₄-halogenoalkoxy, or C₁-C₄-halogenoalkylthio each having 1 to 5 halogen atoms, or
(5) a radical of formula (A5) 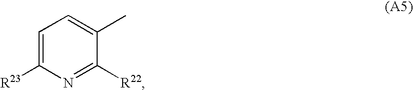
wherein
R²² represents halogen, hydroxyl, cyano, C₁-C₄-alkyl, C₁-C₄-alkoxy, or C₁-C₄-alkylthio; or represents C₁-C₄-halogenoalkyl, C₁-C₄-halogenoalkylthio, or C₁-C₄-halogenoalkoxy each having 1 to 5 halogen atoms, and
R²³ represents hydrogen, halogen, cyano, C₁-C₄-alkyl, C₁-C₄-alkoxy, or C₁-C₄-alkylthio; represents C₁-C₄-halogenoalkyl or C₁-C₄-halogenoalkoxy each having 1 to 5 halogen atoms; or represents C₁-C₄-alkylsulphinyl or C₁-C₄-alkylsulphonyl, or
(6) a radical of formula (A6) 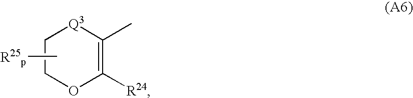
wherein
R²⁴ represents C₁-C₄-alkyl or C₁-C₄-halogenoalkyl having 1 to 5 halogen atoms,
R²⁵ represents C₁-C₄-alkyl,
Q³ represents a sulphur or oxygen atom, SO, SO₂, or CH₂, and
p represents 0, 1, or 2, with the proviso that R²⁵ represents identical or different radicals if p represents 2, or
(7) a radical of formula (A7) 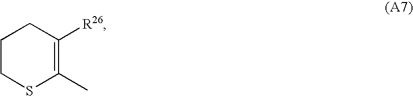
wherein R²⁶ represents C₁-C₄-alkyl or C₁-C₄-halogenoalkyl having 1 to 5 halogen atoms, or
(8) a radical of formula (A8) 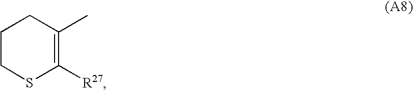
wherein R²⁷ represents C₁-C₄-alkyl or C₁-C₄-halogenoalkyl having 1 to 5 halogen atoms, or
(9) a radical of formula (A9) 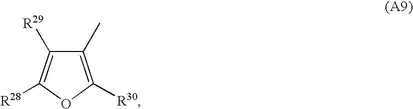
wherein
R²⁸ and R²⁹ independently of one another represent hydrogen, halogen, amino, or C₁-C₄-alkyl; or represent C₁-C₄-halogenoalkyl having 1 to 5 halogen atoms, and
R³⁰ represents hydrogen, halogen, C₁-C₄-alkyl, or C₁-C₄-halogenoalkyl having 1 to 5 halogen atoms, or
(10) a radical of formula (A10) 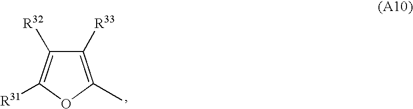
wherein
R³¹ and R³² independently of one another represent hydrogen, halogen, amino, nitro, C₁-C₄-alkyl, or C₁-C₄-halogenoalkyl having 1 to 5 halogen atoms, and
R³³ represents hydrogen, halogen, C₁-C₄-alkyl, or C₁-C₄-halogenoalkyl having 1 to 5 halogen atoms, or
(11) a radical of formula (A11) 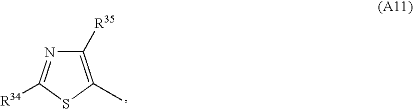
wherein
R³⁴ represents hydrogen, halogen, amino, C₁-C₄-alkylamino, di(C₁-C₄-alkyl)amino, cyano, C₁-C₄-alkyl, or C₁-C₄-halogenoalkyl having 1 to 5 halogen atoms, and
R³⁵ represents halogen, C₁-C₄-alkyl, or C₁-C₄-halogenoalkyl having 1 to 5 halogen atoms, or
(12) a radical of formula (A12) 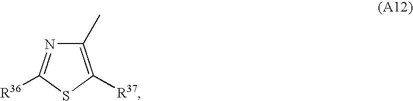
wherein
R³⁶ represents hydrogen, halogen, amino, C₁-C₄-alkylamino, di(C₁-C₄-alkyl)amino, cyano, C₁-C₄-alkyl, or C₁-C₄-halogenoalkyl having 1 to 5 halogen atoms, and
R³⁷ represents halogen, C₁-C₄-alkyl, or C₁-C₄-halogenoalkyl having 1 to 5 halogen atoms, or
(13) a radical of formula (A13) 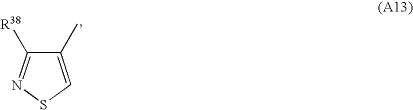
wherein R³⁸ represents halogen, C₁-C₄-alkyl, or C₁-C₄-halogenoalkyl having 1 to 5 halogen atoms, or
(14) a radical of formula (A14) 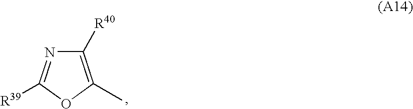
wherein
R³⁹ represents hydrogen or C₁-C₄-alkyl, and
R⁴⁰ represents halogen or C₁-C₄-alkyl, or
(15) a radical of formula (A15) 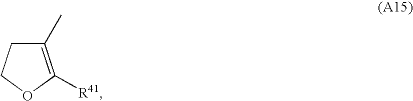
wherein R⁴¹ represents C₁-C₄-alkyl or C₁-C₄-halogenoalkyl having 1 to 5 halogen atoms, or
(16) a radical of formula (A16) 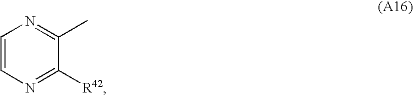
wherein R⁴² represents hydrogen, halogen, C₁-C₄-alkyl, or C₁-C₄-halogenoalkyl having 1 to 5 halogen atoms, or
(17) a radical of formula (A17) 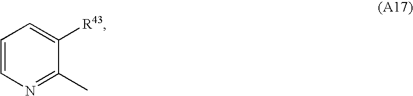
wherein R⁴³ represents halogen, hydroxyl, C₁-C₄-alkyl, C₁-C₄-alkoxy, or C₁-C₄-alkylthio, or represents C₁-C₄-halogenoalkyl, C₁-C₄-halogenoalkylthio, or C₁-C₄-halogenoalkoxy each having 1 to 5 halogen atoms,
with the exception of pyridinylanilides of formula (I-12) in which
R represents hydrogen,
R^{1a} represents halogen; straight-chain or branched alkyl having 1 to 4 carbon atoms; or straight-chain or branched halogenoalkyl having 1 to 4 carbon atoms,
R⁴ represents hydrogen, and
A represents
(i) a radical of formula (A1) 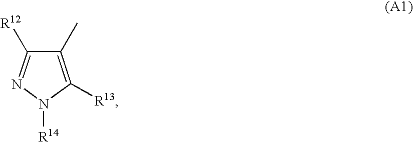
wherein
R¹² represents halogen, C₁-C₄-alkyl, or C₁-C₄-halogenoalkyl,
R¹³ represents hydrogen, and
R¹⁴ represents methyl, or
(ii) a radical of formula (A2) 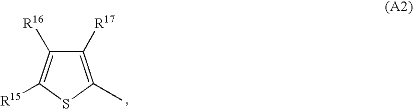
wherein
R¹⁵ and R¹⁶ independently of one another represent hydrogen or C₁-C₄-alkyl, and
R¹⁷ represents halogen, C₁-C₄-alkyl, or C₁-C₄-halogenoalkyl, or
(iii) a radical of formula (A4) 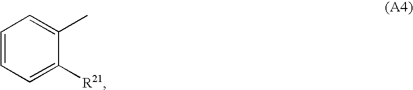
wherein R²¹ represents halogen, C₁-C₄-alkyl, or C₁-C₄-halogenoalkyl, or
(iv) a radical of formula (A5) 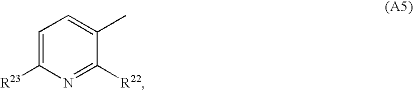
wherein
R²² represents halogen, and
R²³ represents hydrogen, or
(v) a radical of formula (A6) 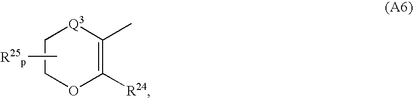
wherein
R²⁴ represents methyl,
Q³ represents a sulphur atom or CH₂, and
p represents 0, or
(vi) a radical of formula (A9) 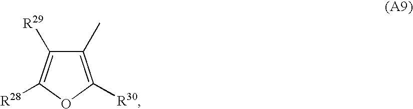
wherein
R²⁸ and R²⁹ independently of one another each represent hydrogen or C₁-C₄-alkyl, and
R³⁰ represents methyl, or
(vii) a radical of formula (A11) 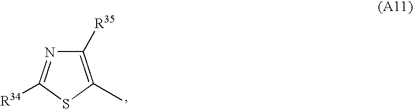
wherein
R³⁴ represents hydrogen or C₁-C₄-alkyl, and
R³⁵ represents halogen, C₁-C₄-alkyl, or C₁-C₄-halogenoalkyl, or
(viii) a radical of formula (A16) 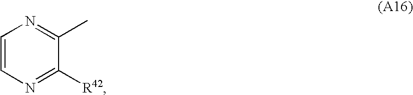
wherein R⁴² represents halogen.

28. A pyridinylanilide of formula (I-13) 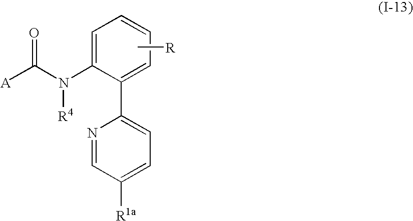
in which
R represents hydrogen, fluorine, chlorine, methyl, or trifluoromethyl;
R^{1a} represents halogen, cyano, nitro, amino, hydroxyl, formyl, carboxyl, carbamoyl, or thiocarbamoyl; represents straight-chain or branched alkyl, hydroxyalkyl, oxoalkyl, alkoxy, alkoxyalkyl, alkylthioalkyl, dialkoxyalkyl, alkylthio, alkylsulfinyl, or alkylsulfonyl having in each case 1 to 8 carbon atoms in the respective alkyl moiety; represents straight-chain or branched alkenyl or alkenyloxy having in each case 2 to 6 carbon atoms; represents straight-chain or branched halogenoalkyl, halogenoalkoxy, halogenoalkylthio, halogenoalkylsulfinyl, or halogenoalkylsulfonyl having in each case 1 to 6 carbon atoms and 1 to 13 identical or different halogen atoms; represents straight-chain or branched halogenoalkenyl or halogenoalkenyloxy having in each case 2 to 6 carbon atoms and 1 to 11 identical or different halogen atoms; represents straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, or dialkylaminocarbonyloxy having 1 to 6 carbon atoms in the respective hydrocarbon chain; represents alkenylcarbonyl or alkynylcarbonyl having 2 to 6 carbon atoms in the respective hydrocarbon chain; represents cycloalkyl or cycloalkyloxy having in each case 3 to 6 carbon atoms; represents the grouping —C(Q¹)=N-Q², wherein
Q¹ represents hydrogen, hydroxyl, C₁-C₄-alkyl, C₁-C₄-halogenoalkyl having 1 to 9 identical or different halogen atoms, or C₃-C₆-cycloalkyl, and
Q² represents hydroxyl, amino, methylamino, phenyl, or benzyl; or represents C₁-C₄-alkyl or C₁-C₄-alkoxy, each of which is optionally substituted by halogen, cyano, hydroxyl, C₁-C₄-alkoxy, C₁-C₄-alkylthio, C₁-C₄-alkylamino, di(C₁-C₄-alkyl)amino, or phenyl; or represents C₂-C₄-alkenyloxy or C₂-C₄-alkynyloxy;
represents phenyl, phenoxy, phenylthio, benzoyl, benzoylethenyl, cinnamoyl, or heterocyclyl; or represents phenylalkyl, phenylalkyloxy, phenylalkylthio, or heterocyclylalkyl having in each case 1 to 3 carbon atoms in the respective alkyl moieties, each of which is optionally mono- to tri-substituted, identically or differently, in the ring moiety by halogen or straight-chain or branched C₁-C₄-alkyl or C₁-C₄-alkoxy;
R⁴ represents hydrogen, C₁-C₈-alkyl, C₁-C₆-alkylsulfinyl, C₁-C₆-alkylsulfonyl, C₁-C₄-alkoxy-C₁-C₄-alkyl, or C₃-C₈-cycloalkyl; represents C₁-C₆-halogenoalkyl, C₁-C₄-halogenoalkylthio, C₁-C₄-halogenoalkylsulfinyl, C₁-C₄-halogenoalkylsulfonyl, halogeno-C₁-C₄-alkoxy-C₁-C₄-alkyl, or C₃-C₈-halogenocycloalkyl having in each case 1 to 9 fluorine-, chlorine-, and/or bromine atoms; represents formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C₃-halogenoalkyl)carbonyl-C₁-C₃-alkyl, or (C₁-C₃-halogenoalkoxy)carbonyl-C₁-C₃-alkyl having in each case 1 to 7 fluorine-, chlorine-, and/or bromine atoms; represents (C₁-C₃-alkyl)carbonyl-C₁-C₃-halogenoalkyl or (C₁-C₃-alkoxy)carbonyl-C₁-C₃-halogenoalkyl having in each case 1 to 6 fluorine-, chlorine- ,and/or bromine atoms represents (C₁-C₃-halogenoalkyl)carbonyl-C₁-C₃-halogenoalkyl, (C₁-C₃-halogenoalkoxy)-carbonyl-C₁-C₃-halogenoalkyl having in each case 1 to 13 fluorine-, chlorine-, and/or bromine atoms; or represents —COR⁵, —CONR⁶R⁷, or —CH₂NR⁸R⁹,
R⁵ represents hydrogen, C₁-C₈-alkyl, C₁-C₈-alkoxy, C₁-C₄-alkoxy-C₁-C₄-alkyl, or C₃-C₈-cycloalkyl; represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms; or represents —COR¹⁰,
R⁶ and R⁷ independently of one another represent hydrogen, C₁-C₈-alkyl, C₁-C₄-alkoxy-C₁-C₄-alkyl, or C₃-C₈-cycloalkyl; or represent C₁-C₈-halogenoalkyl, halogeno-C₁-C₄-alkoxy-C₁-C₄-alkyl, or C₃-C₈-halogenocycloalkyl having in each case 1 to 9 fluorine-, chlorine-, and/or bromine atoms; or R⁶ and R⁷ together with the nitrogen atom to which they are attached represent a saturated 5- to 8-membered heterocycle, wherein the heterocycle optionally has 1 or 2 additional non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R⁸ and R⁹ independently of one another represent hydrogen, C₁-C₈-alkyl, or C₃-C₈-cycloalkyl; or represent C₁-C₈-halogenoalkyl, C₃-C₈-halogenocycloalkyl having in each case 1 to 9 fluorine-, chlorine- and/or bromine atoms; or R⁸ and R⁹ together with the nitrogen atom to which they are attached, represent a saturated 5- to 8-membered heterocycle, wherein the heterocycle optionally has 1 or 2 additional, non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R¹⁰ represents hydrogen, C₁-C₈-alkyl, C₁-C₈-alkoxy, C₁-C₄-alkoxy-C₁-C₄-alkyl, or C₃-C₈-cycloalkyl; or represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms,
R¹¹ represents hydrogen or C₁-C₆-alkyl, and
A represents
(1) a radical of formula (A1) 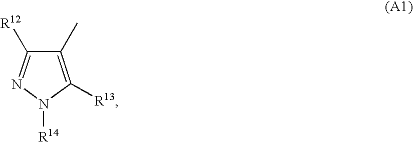
wherein
R¹² represents hydrogen, cyano, halogen, nitro, C₁-C₄-alkyl, C₁-C₄-alkoxy, C₁-C₄-alkylthio, or C₃-C₆-cycloalkyl; represents C₁-C₄-halogenoalkyl, C₁-C₄-halogenoalkoxy, or C₁-C₄-halogenoalkylthio each having 1 to 5 halogen atoms; or represents aminocarbonyl or aminocarbonyl-C₁-C₄-alkyl,
R¹³ represents hydrogen, halogen, cyano, C₁-C₄-alkyl, C₁-C₄-alkoxy, or C₁-C₄-alkylthio, and
R¹⁴ represents hydrogen, C₁-C₄-alkyl, hydroxy-C₁-C₄-alkyl, C₂-C₆-alkenyl, C₃-C₆-cycloalkyl, C₁-C₄-alkylthio-C₁-C₄-alkyl, or C₁-C₄-alkoxy-C₁-C₄-alkyl; represents C₁-C₄-halogenoalkyl, C₁-C₄-halogenoalkylthio-C₁-C₄-alkyl, or C₁ -C₄-halogenoalkoxy-C₁-C₄-alkyl each having 1 to 5 halogen atoms; or represents phenyl, or
(2) a radical of formula (A2) 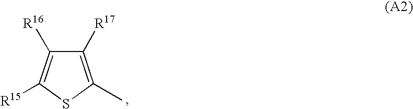
wherein
R¹⁵ and R¹⁶ independently of one another represent hydrogen, halogen, C₁-C₄-alkyl, or C₁-C₄-halogenoalkyl having 1 to 5 halogen atoms, and
R¹⁷ represents halogen, cyano or C₁-C₄-alkyl; or represents C₁-C₄-halogenoalkyl or C₁-C₄-halogenoalkoxy each having 1 to 5 halogen atoms, or
(3) a radical of formula (A3) 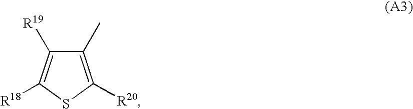
wherein
R¹⁸ and R¹⁹ independently of one another represent hydrogen, halogen, C₁-C₄-alkyl, or C₁-C₄-halogenoalkyl having 1 to 5 halogen atoms, and
R²⁰ represents hydrogen, halogen, C₁-C₄-alkyl, or C₁-C₄-halogenoalkyl having 1 to 5 halogen atoms, or
(4) a radical of formula (A4) 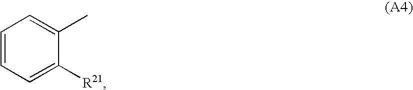
wherein R²¹ represents hydrogen, halogen, hydroxyl, cyano, or C₁-C₆-alkyl; or represents C₁-C₄-halogenoalkyl, C₁-C₄-halogenoalkoxy, or C₁-C₄-halogenoalkylthio each having 1 to 5 halogen atoms, or
(5) a radical of formula (A5) 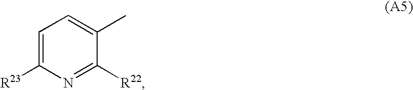
wherein
R²² represents halogen, hydroxyl, cyano, C₁-C₄-alkyl, C₁-C₄-alkoxy, or C₁-C₄-alkylthio; or represents C₁-C₄-halogenoalkyl, C₁-C₄-halogenoalkylthio, or C₁-C₄-halogenoalkoxy each having 1 to 5 halogen atoms, and
R²³ represents hydrogen, halogen, cyano, C₁-C₄-alkyl, C₁-C₄-alkoxy, or C₁-C₄-alkylthio; represents C₁-C₄-halogenoalkyl or C₁-C₄-halogenoalkoxy each having 1 to 5 halogen atoms; or represents C₁-C₄-alkylsulphinyl or C₁-C₄-alkylsulphonyl, or
(6) a radical of formula (A6) 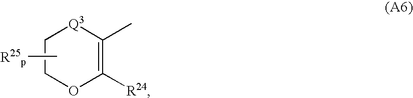
wherein
R²⁴ represents C₁-C₄-alkyl or C₁-C₄-halogenoalkyl having 1 to 5 halogen atoms,
R²⁵ represents C₁-C₄-alkyl,
Q³ represents a sulphur or oxygen atom, SO, SO₂, or CH₂, and
p represents 0, 1, or 2, with the proviso that R²⁵ represents identical or different radicals if p represents 2, or
(7) a radical of formula (A7) 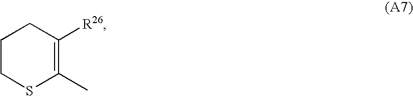
wherein R²⁶ represents C₁-C₄-alkyl or C₁-C₄-halogenoalkyl having 1 to 5 halogen atoms, or
(8) a radical of formula (A8) 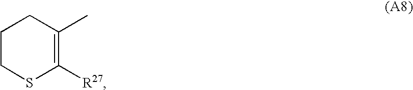
wherein R²⁷ represents C₁-C₄-alkyl or C₁-C₄-halogenoalkyl having 1 to 5 halogen atoms, or
(9) a radical of formula (A9) 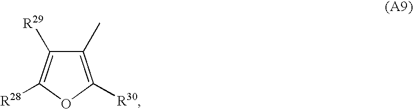
wherein
R²⁸ and R²⁹ independently of one another represent hydrogen, halogen, amino, or C₁-C₄-alkyl; or represent C₁-C₄-halogenoalkyl having 1 to 5 halogen atoms, and
R³⁰ represents hydrogen, halogen, C₁-C₄-alkyl, or C₁-C₄-halogenoalkyl having 1 to 5 halogen atoms, or
(10) a radical of formula (A10) 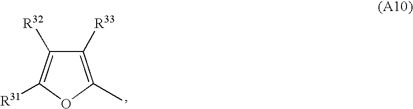
wherein
R³¹ and R³² independently of one another represent hydrogen, halogen, amino, nitro, C₁-C₄-alkyl, or C₁-C₄-halogenoalkyl having 1 to 5 halogen atoms, and
R³³ represents hydrogen, halogen, C₁-C₄-alkyl, or C₁-C₄-halogenoalkyl having 1 to 5 halogen atoms, or
(11) a radical of formula (A11) 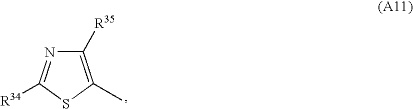
wherein
R³⁴ represents hydrogen, halogen, amino, C₁-C₄-alkylamino, di(C₁-C₄-alkyl)amino, cyano, C₁-C₄-alkyl, or C₁-C₄-halogenoalkyl having 1 to 5 halogen atoms, and
R³⁵ represents halogen, C₁-C₄-alkyl, or C₁-C₄-halogenoalkyl having 1 to 5 halogen atoms, or
(12) a radical of formula (A12) 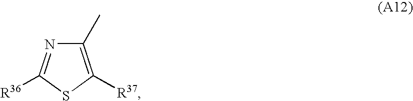
wherein
R³⁶ represents hydrogen, halogen, amino, C₁-C₄-alkylamino, di(C₁-C₄-alkyl)amino, cyano, C₁-C₄-alkyl, or C₁-C₄-halogenoalkyl having 1 to 5 halogen atoms, and
R³⁷ represents halogen, C₁-C₄-alkyl, or C₁-C₄-halogenoalkyl having 1 to 5 halogen atoms, or
(13) a radical of formula (A13) 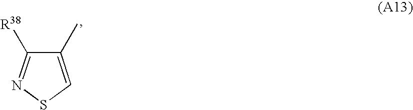
wherein R³⁸ represents halogen, C₁-C₄-alkyl, or C₁-C₄-halogenoalkyl having 1 to 5 halogen atoms, or
(14) a radical of formula (A14) 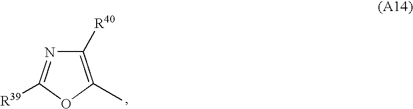
wherein
R³⁹ represents hydrogen or C₁-C₄-alkyl, and
R⁴⁰ represents halogen or C₁-C₄-alkyl, or
(15) a radical of formula (A15) 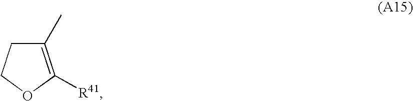
wherein R⁴¹ represents C₁-C₄-alkyl or C₁-C₄-halogenoalkyl having 1 to 5 halogen atoms, or
(16) a radical of formula (A16) 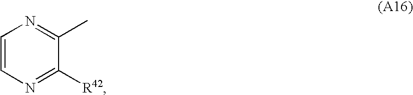
wherein R⁴² represents hydrogen, halogen, C₁-C₄-alkyl, or C₁-C₄-halogenoalkyl having 1 to 5 halogen atoms, or
(17) a radical of formula (A17) 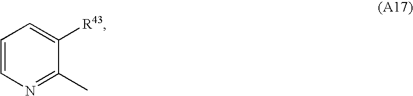
wherein R⁴³ represents halogen, hydroxyl, C₁-C₄-alkyl, C₁-C₄-alkoxy, or C₁-C₄-alkylthio, or represents C₁-C₄-halogenoalkyl, C₁-C₄-halogenoalkylthio, or C₁-C₄-halogenoalkoxy each having 1 to 5 halogen atoms,
with the exception of pyridinylanilides of formula (I-13) in which
R represents hydrogen,
R^{1a} represents halogen; straight-chain or branched alkyl having 1 to 4 carbon atoms; or straight-chain or branched halogenoalkyl having 1 to 4 carbon atoms,
R⁴ represents hydrogen, and
A represents
(i) a radical of formula (A1) 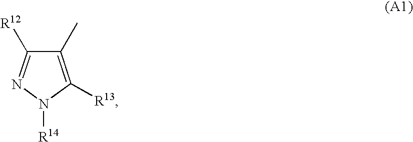
wherein
R¹² represents halogen, C₁-C₄-alkyl, or C₁-C₄-halogenoalkyl,
R¹³ represents hydrogen, and
R¹⁴ represents methyl, or
(ii) a radical of formula (A2) 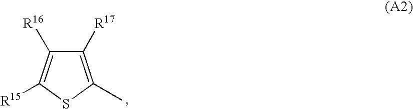
wherein
R¹⁵ and R¹⁶ independently of one another represent hydrogen or C₁-C₄-alkyl, and
R¹⁷ represents halogen, C₁-C₄-alkyl, or C₁-C₄-halogenoalkyl, or
(iii) a radical of formula (A4) 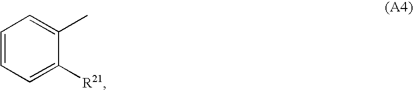
wherein R²¹ represents halogen, C₁-C₄-alkyl, or C₁-C₄-halogenoalkyl, or
(iv) a radical of formula (A5) 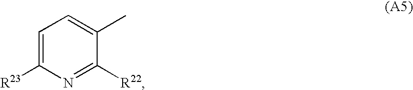
wherein
R²² represents halogen, and
R²³ represents hydrogen, or
(v) a radical of formula (A6) 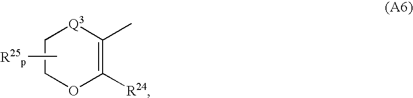
wherein
R²⁴ represents methyl,
Q³ represents a sulphur atom or CH₂, and
p represents 0, or
(vi) a radical of formula (A9) 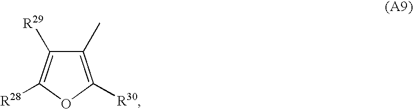
wherein
R²⁸ and R²⁹ independently of one another each represent hydrogen or C₁-C₄-alkyl, and
R³⁰ represents methyl, or
(vii) a radical of formula (A11) 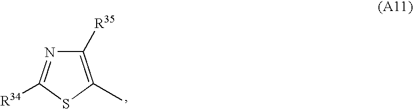
wherein
R³⁴ represents hydrogen or C₁-C₄-alkyl, and
R³⁵ represents halogen, C₁-C₄-alkyl, or C₁-C₄-halogenoalkyl, or
(viii) a radical of formula (A16) 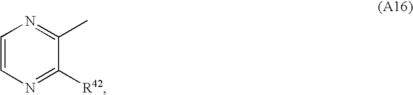
wherein R⁴² represents halogen.

29. A pyridinylanilide of formula (I-14) 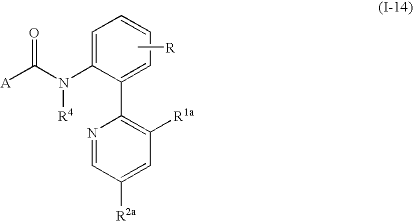
in which
R represents hydrogen, fluorine, chlorine, methyl, or trifluoromethyl;
R^{1a} and R^{2a} independently of one another represent halogen, cyano, nitro, amino, hydroxyl, formyl, carboxyl, carbamoyl, or thiocarbamoyl; represent straight-chain or branched alkyl, hydroxyalkyl, oxoalkyl, alkoxy, alkoxyalkyl, alkylthioalkyl, dialkoxyalkyl, alkylthio, alkylsulfinyl, or alkylsulfonyl having in each case 1 to 8 carbon atoms in the respective alkyl moiety; represent straight-chain or branched alkenyl or alkenyloxy having in each case 2 to 6 carbon atoms; represent straight-chain or branched halogenoalkyl, halogenoalkoxy, halogenoalkylthio, halogenoalkylsulfinyl, or halogenoalkylsulfonyl having in each case 1 to 6 carbon atoms and 1 to 13 identical or different halogen atoms; represent straight-chain or branched halogenoalkenyl or halogenoalkenyloxy having in each case 2 to 6 carbon atoms and 1 to 11 identical or different halogen atoms; represent straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, or dialkylaminocarbonyloxy having 1 to 6 carbon atoms in the respective hydrocarbon chain; represent alkenylcarbonyl or alkynylcarbonyl having 2 to 6 carbon atoms in the respective hydrocarbon chain; represent cycloalkyl or cycloalkyloxy having in each case 3 to 6 carbon atoms; represents the group —C(Q¹)=N-Q², wherein
Q¹ represents hydrogen, hydroxyl, C₁-C₄-alkyl, C₁-C₄-halogenoalkyl having 1 to 9 identical or different halogen atoms, or C₃-C₆-cycloalkyl, and
Q² represents hydroxyl, amino, methylamino, phenyl, or benzyl; or represents C₁-C₄-alkyl or C₁-C₄-alkoxy, each of which is optionally substituted by halogen, cyano, hydroxyl, C₁-C₄-alkoxy, C₁-C₄-alkylthio, C₁-C₄-alkylamino, di(C₁-C₄-alkyl)amino, or phenyl; or represents C₂-C₄-alkenyloxy or C₂-C₄-alkynyloxy;
represent phenyl, phenoxy, phenylthio, benzoyl, benzoylethenyl, cinnamoyl, or heterocyclyl; or represent phenylalkyl, phenylalkyloxy, phenylalkylthio, or heterocyclylalkyl having in each case 1 to 3 carbon atoms in the respective alkyl moieties, each of which is optionally mono- to tri-substituted, identically or differently, in the ring moiety by halogen or straight-chain or branched C₁-C₄-alkyl or C₁-C₄-alkoxy;
R⁴ represents hydrogen, C₁-C₈-alkyl, C₁-C₆-alkylsulfinyl, C₁-C₆-alkylsulfonyl, C₁-C₄-alkoxy-C₁-C₄-alkyl, or C₃-C₈-cycloalkyl; represents C₁-C₆-halogenoalkyl, C₁-C₄-halogenoalkylthio, C₁-C₄-halogenoalkylsulfinyl, C₁-C₄-halogenoalkylsulfonyl, halogeno-C₁-C₄-alkoxy-C₁-C₄-alkyl, or C₃-C₈-halogenocycloalkyl having in each case 1 to 9 fluorine-, chlorine-, and/or bromine atoms; represents formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C₃-halogenoalkyl)carbonyl-C₁-C₃-alkyl, or (C₁-C₃-halogenoalkoxy)carbonyl-C₁-C₃-alkyl having in each case 1 to 7 fluorine-, chlorine-, and/or bromine atoms; represents (C₁-C₃-alkyl)carbonyl-C₁-C₃-halogenoalkyl or (C₁-C₃-alkoxy)carbonyl-C₁-C₃-halogenoalkyl having in each case 1 to 6 fluorine-, chlorine- ,and/or bromine atoms represents (C₁-C₃-halogenoalkyl)carbonyl-C₁-C₃-halogenoalkyl, (C₁-C₃-halogenoalkoxy)-carbonyl-C₁-C₃-halogenoalkyl having in each case 1 to 13 fluorine-, chlorine-, and/or bromine atoms; or represents —COR⁵, —CONR⁶R⁷, or —CH₂NR⁸R⁹,
R⁵ represents hydrogen, C₁-C₈-alkyl, C₁-C₈-alkoxy, C₁-C₄-alkoxy-C₁-C₄-alkyl, or C₃-C₈-cycloalkyl; represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms; or represents —COR¹⁰,
R⁶ and R⁷ independently of one another represent hydrogen, C₁-C₈-alkyl, C₁-C₄-alkoxy-C₁-C₄-alkyl, or C₃-C₈-cycloalkyl; or represent C₁-C₈-halogenoalkyl, halogeno-C₁-C₄-alkoxy-C₁-C₄-alkyl, or C₃-C₈-halogenocycloalkyl having in each case 1 to 9 fluorine-, chlorine-, and/or bromine atoms; or R⁶ and R⁷ together with the nitrogen atom to which they are attached represent a saturated 5- to 8-membered heterocycle, wherein the heterocycle optionally has 1 or 2 additional non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R⁸ and R⁹ independently of one another represent hydrogen, C₁-C₈-alkyl, or C₃-C₈-cycloalkyl; or represent C₁-C₈-halogenoalkyl, C₃-C₈-halogenocycloalkyl having in each case 1 to 9 fluorine-, chlorine- and/or bromine atoms; or R⁸ and R⁹ together with the nitrogen atom to which they are attached, represent a saturated 5- to 8-membered heterocycle, wherein the heterocycle optionally has 1 or 2 additional, non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R¹⁰ represents hydrogen, C₁-C₈-alkyl, C₁-C₈-alkoxy, C₁-C₄-alkoxy-C₁-C₄-alkyl, or C₃-C₈-cycloalkyl; or represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms,
R¹¹ represents hydrogen or C₁-C₆-alkyl, and
A represents
(1) a radical of formula (A1) 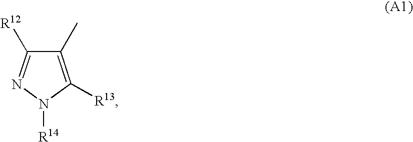
wherein
R¹² represents hydrogen, cyano, halogen, nitro, C₁-C₄-alkyl, C₁-C₄-alkoxy, C₁-C₄-alkylthio, or C₃-C₆-cycloalkyl; represents C₁-C₄-halogenoalkyl, C₁-C₄-halogenoalkoxy, or C₁-C₄-halogenoalkylthio each having 1 to 5 halogen atoms; or represents aminocarbonyl or aminocarbonyl-C₁-C₄-alkyl,
R¹³ represents hydrogen, halogen, cyano, C₁-C₄-alkyl, C₁-C₄-alkoxy, or C₁-C₄-alkylthio, and
R¹⁴ represents hydrogen, C₁-C₄-alkyl, hydroxy-C₁-C₄-alkyl, C₂-C₆-alkenyl, C₃-C₆-cycloalkyl, C₁-C₄-alkylthio-C₁-C₄-alkyl, or C₁-C₄-alkoxy-C₁-C₄-alkyl; represents C₁-C₄-halogenoalkyl, C₁-C₄-halogenoalkylthio-C₁-C₄-alkyl, or C₁-C₄-halogenoalkoxy-C₁-C₄-alkyl each having 1 to 5 halogen atoms; or represents phenyl, or
(2) a radical of formula (A2) 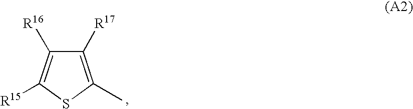
wherein
R¹⁵ and R¹⁶ independently of one another represent hydrogen, halogen, C₁-C₄-alkyl, or C₁-C₄-halogenoalkyl having 1 to 5 halogen atoms, and
R¹⁷ represents halogen, cyano or C₁-C₄-alkyl; or represents C₁-C₄-halogenoalkyl or C₁-C₄-halogenoalkoxy each having 1 to 5 halogen atoms, or
(3) a radical of formula (A3) 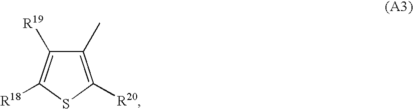
wherein
R¹⁸ and R¹⁹ independently of one another represent hydrogen, halogen, C₁-C₄-alkyl, or C₁-C₄-halogenoalkyl having 1 to 5 halogen atoms, and
R²⁰ represents hydrogen, halogen, C₁-C₄-alkyl, or C₁-C₄-halogenoalkyl having 1 to 5 halogen atoms, or
(4) a radical of formula (A4) 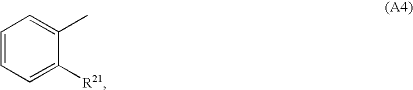
wherein R²¹ represents hydrogen, halogen, hydroxyl, cyano, or C₁-C₆-alkyl; or represents C₁-C₄-halogenoalkyl, C₁-C₄-halogenoalkoxy, or C₁-C₄-halogenoalkylthio each having 1 to 5 halogen atoms, or
(5) a radical of formula (A5) 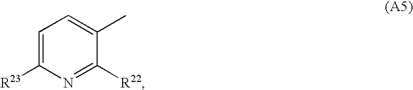
wherein
R²² represents halogen, hydroxyl, cyano, C₁-C₄-alkyl, C₁-C₄-alkoxy, or C₁-C₄-alkylthio; or represents C₁-C₄-halogenoalkyl, C₁-C₄-halogenoalkylthio, or C₁-C₄-halogenoalkoxy each having 1 to 5 halogen atoms, and
R²³ represents hydrogen, halogen, cyano, C₁-C₄-alkyl, C₁-C₄-alkoxy, or C₁-C₄-alkylthio; represents C₁-C₄-halogenoalkyl or C₁-C₄-halogenoalkoxy each having 1 to 5 halogen atoms; or represents C₁-C₄-alkylsulphinyl or C₁-C₄-alkylsulphonyl, or
(6) a radical of formula (A6) 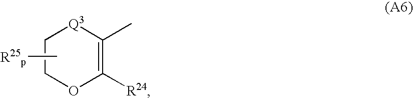
wherein
R²⁴ represents C₁-C₄-alkyl or C₁-C₄-halogenoalkyl having 1 to 5 halogen atoms,
R²⁵ represents C₁-C₄-alkyl,
Q³ represents a sulphur or oxygen atom, SO, SO₂, or CH₂, and
p represents 0, 1, or 2, with the proviso that R²⁵ represents identical or different radicals if p represents 2, or
(7) a radical of formula (A7) 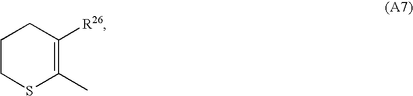
wherein R²⁶ represents C₁-C₄-alkyl or C₁-C₄-halogenoalkyl having 1 to 5 halogen atoms, or
(8) a radical of formula (A8) 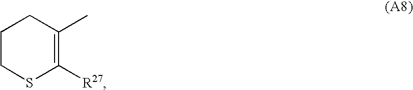
wherein R²⁷ represents C₁-C₄-alkyl or C₁-C₄-halogenoalkyl having 1 to 5 halogen atoms, or
(9) a radical of formula (A9) 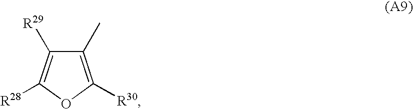
wherein
R²⁸ and R²⁹ independently of one another represent hydrogen, halogen, amino, or C₁-C₄-alkyl; or represent C₁-C₄-halogenoalkyl having 1 to 5 halogen atoms, and
R³⁰ represents hydrogen, halogen, C₁-C₄-alkyl, or C₁-C₄-halogenoalkyl having 1 to 5 halogen atoms, or
(10) a radical of formula (A10) 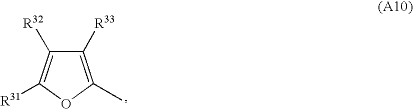
wherein
R³¹ and R³² independently of one another represent hydrogen, halogen, amino, nitro, C₁-C₄-alkyl, or C₁-C₄-halogenoalkyl having 1 to 5 halogen atoms, and
R³³ represents hydrogen, halogen, C₁-C₄-alkyl, or C₁-C₄-halogenoalkyl having 1 to 5 halogen atoms, or
(11) a radical of formula (A11) 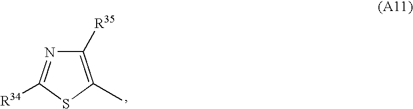
wherein
R³⁴ represents hydrogen, halogen, amino, C₁-C₄-alkylamino, di(C₁-C₄-alkyl)amino, cyano, C₁-C₄-alkyl, or C₁-C₄-halogenoalkyl having 1 to 5 halogen atoms, and
R³⁵ represents halogen, C₁-C₄-alkyl, or C₁-C₄-halogenoalkyl having 1 to 5 halogen atoms, or
(12) a radical of formula (A12) 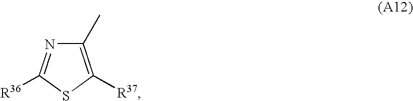
wherein
R³⁶ represents hydrogen, halogen, amino, C₁-C₄-alkylamino, di(C₁-C₄-alkyl)amino, cyano, C₁-C₄-alkyl, or C₁-C₄-halogenoalkyl having 1 to 5 halogen atoms, and
R³⁷ represents halogen, C₁-C₄-alkyl, or C₁-C₄-halogenoalkyl having 1 to 5 halogen atoms, or
(13) a radical of formula (A13) 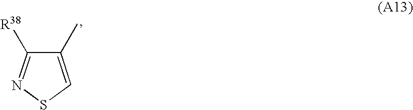
wherein R³⁸ represents halogen, C₁-C₄-alkyl, or C₁-C₄-halogenoalkyl having 1 to 5 halogen atoms, or
(14) a radical of formula (A14) 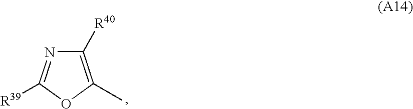
wherein
R³⁹ represents hydrogen or C₁-C₄-alkyl, and
R⁴⁰ represents halogen or C₁-C₄-alkyl, or
(15) a radical of formula (A15) 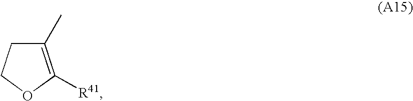
wherein R⁴¹ represents C₁-C₄-alkyl or C₁-C₄-halogenoalkyl having 1 to 5 halogen atoms, or
(16) a radical of formula (A16) 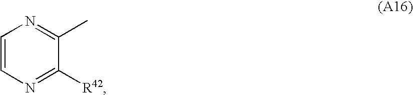
wherein R⁴² represents hydrogen, halogen, C₁-C₄-alkyl, or C₁-C₄-halogenoalkyl having 1 to 5 halogen atoms, or
(17) a radical of formula (A17) 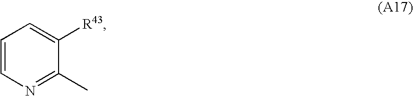
wherein R⁴³ represents halogen, hydroxyl, C₁-C₄-alkyl, C₁-C₄-alkoxy, or C₁-C₄-alkylthio, or represents C₁-C₄-halogenoalkyl, C₁-C₄-halogenoalkylthio, or C₁-C₄-halogenoalkoxy each having 1 to 5 halogen atoms,
with the exception of pyridinylanilides of formula (I-14) in which
R represents hydrogen,
R^{1a} and R^{2a} independently of one another each represents halogen; straight-chain or branched alkyl having 1 to 4 carbon atoms; or straight-chain or branched halogenoalkyl having 1 to 4 carbon atoms,
R⁴ represents hydrogen, and
A represents
(i) a radical of formula (A1) 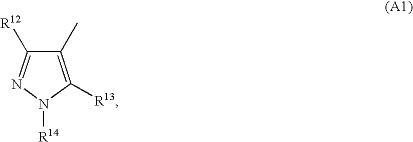
wherein
R¹² represents halogen, C₁-C₄-alkyl, or C₁-C₄-halogenoalkyl,
R¹³ represents hydrogen, and
R¹⁴ represents methyl, or
(ii) a radical of formula (A2) 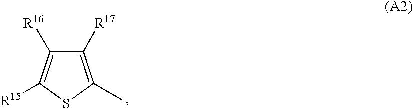
wherein
R¹⁵ and R¹⁶ independently of one another represent hydrogen or C₁-C₄-alkyl, and
R¹⁷ represents halogen, C₁-C₄-alkyl, or C₁-C₄-halogenoalkyl, or
(iii) a radical of formula (A4) 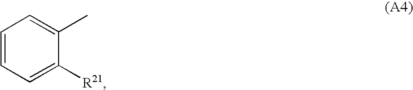
wherein R represents halogen, C₁-C₄-alkyl, or C₁-C₄-halogenoalkyl, or
(iv) a radical of formula (A5) 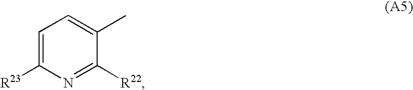
wherein
R²² represents halogen, and
R²³ represents hydrogen, or
(v) a radical of formula (A6) 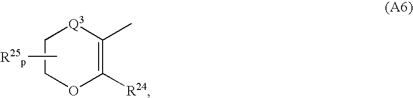
wherein
R²⁴ represents methyl,
Q³ represents a sulphur atom or CH₂, and
p represents 0, or
(vi) a radical of formula (A9) 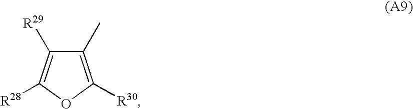
wherein
R²⁸ and R²⁹ independently of one another each represent hydrogen or C₁-C₄-alkyl, and
R³⁰ represents methyl, or
(vii) a radical of formula (A11) 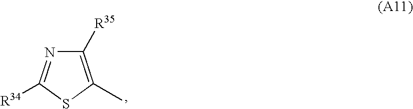
wherein
R³⁴ represents hydrogen or C₁-C₄-alkyl, and
R³⁵ represents halogen, C₁-C₄-alkyl, or C₁-C₄-halogenoalkyl, or
(viii) a radical of formula (A16) 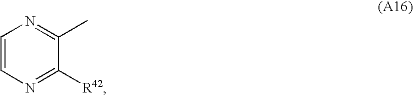
wherein R⁴² represents halogen.

30. A pyridinylanilide of formula (I-15) 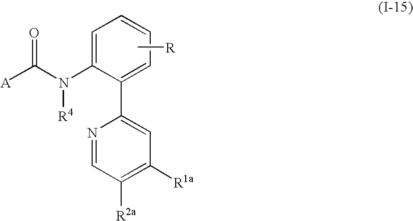
in which
R represents hydrogen, fluorine, chlorine, methyl, or trifluoromethyl;
R^{1a} and R^{2a} independently of one another represent halogen, cyano, nitro, amino, hydroxyl, formyl, carboxyl, carbamoyl, or thiocarbamoyl; represent straight-chain or branched alkyl, hydroxyalkyl, oxoalkyl, alkoxy, alkoxyalkyl, alkylthioalkyl, dialkoxyalkyl, alkylthio, alkylsulfinyl, or alkylsulfonyl having in each case 1 to 8 carbon atoms in the respective alkyl moiety; represent straight-chain or branched alkenyl or alkenyloxy having in each case 2 to 6 carbon atoms; represent straight-chain or branched halogenoalkyl, halogenoalkoxy, halogenoalkylthio, halogenoalkylsulfinyl, or halogenoalkylsulfonyl having in each case 1 to 6 carbon atoms and 1 to 13 identical or different halogen atoms; represent straight-chain or branched halogenoalkenyl or halogenoalkenyloxy having in each case 2 to 6 carbon atoms and 1 to 11 identical or different halogen atoms; represent straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, or dialkylaminocarbonyloxy having 1 to 6 carbon atoms in the respective hydrocarbon chain; represent alkenylcarbonyl or alkynylcarbonyl having 2 to 6 carbon atoms in the respective hydrocarbon chain; represent cycloalkyl or cycloalkyloxy having in each case 3 to 6 carbon atoms; represent the group —C(Q¹)=N-Q², wherein
Q¹ represents hydrogen, hydroxyl, C₁-C₄-alkyl, C₁-C₄-halogenoalkyl having 1 to 9 identical or different halogen atoms, or C₃-C₆-cycloalkyl, and
Q² represents hydroxyl, amino, methylamino, phenyl, or benzyl; or represents C₁-C₄-alkyl or C₁-C₄-alkoxy, each of which is optionally substituted by halogen, cyano, hydroxyl, C₁-C₄-alkoxy, C₁-C₄-alkylthio, C₁-C₄-alkylamino, di(C₁-C₄-alkyl)amino, or phenyl; or represents C₂-C₄-alkenyloxy or C₂-C₄-alkynyloxy;
represent phenyl, phenoxy, phenylthio, benzoyl, benzoylethenyl, cinnamoyl, or heterocyclyl; or represent phenylalkyl, phenylalkyloxy, phenylalkylthio, or heterocyclylalkyl having in each case 1 to 3 carbon atoms in the respective alkyl moieties, each of which is optionally mono- to tri-substituted, identically or differently, in the ring moiety by halogen or straight-chain or branched C₁-C₄-alkyl or C₁-C₄-alkoxy;
R⁴ represents hydrogen, C₁-C₈-alkyl, C₁-C₆-alkylsulfinyl, C₁-C₆-alkylsulfonyl, C₁-C₄-alkoxy-C₁-C₄-alkyl, or C₃-C₈-cycloalkyl; represents C₁-C₆-halogenoalkyl, C₁-C₄-halogenoalkylthio, C₁-C₄-halogenoalkylsulfinyl, C₁-C₄-halogenoalkylsulfonyl, halogeno-C₁-C₄-alkoxy-C₁-C₄-alkyl, or C₃-C₈-halogenocycloalkyl having in each case 1 to 9 fluorine-, chlorine-, and/or bromine atoms; represents formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C₃-halogenoalkyl)carbonyl-C₁-C₃-alkyl, or (C₁-C₃-halogenoalkoxy)carbonyl-C₁-C₃-alkyl having in each case 1 to 7 fluorine-, chlorine-, and/or bromine atoms; represents (C₁-C₃-alkyl)carbonyl-C₁-C₃-halogenoalkyl or (C₁-C₃-alkoxy)carbonyl-C₁-C₃-halogenoalkyl having in each case 1 to 6 fluorine-, chlorine- ,and/or bromine atoms represents (C₁-C₃-halogenoalkyl)carbonyl-C₁-C₃-halogenoalkyl, (C₁-C₃-halogenoalkoxy)-carbonyl-C₁-C₃-halogenoalkyl having in each case 1 to 13 fluorine-, chlorine-, and/or bromine atoms; or represents —COR⁵, —CONR⁶R⁷, or —CH₂NR⁸R⁹,
R⁵ represents hydrogen, C₁-C₈-alkyl, C₁-C₈-alkoxy, C₁-C₄-alkoxy-C₁-C₄-alkyl, or C₃-C₈-cycloalkyl; represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms; or represents —COR¹⁰,
R⁶ and R⁷ independently of one another represent hydrogen, C₁-C₈-alkyl, C₁-C₄-alkoxy-C₁-C₄-alkyl, or C₃-C₈-cycloalkyl; or represent C₁-C₈-halogenoalkyl, halogeno-C₁-C₄-alkoxy-C₁-C₄-alkyl, or C₃-C₈-halogenocycloalkyl having in each case 1 to 9 fluorine-, chlorine-, and/or bromine atoms; or R⁶ and R⁷ together with the nitrogen atom to which they are attached represent a saturated 5- to 8-membered heterocycle, wherein the heterocycle optionally has 1 or 2 additional non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R⁸ and R⁹ independently of one another represent hydrogen, C₁-C₈-alkyl, or C₃-C₈-cycloalkyl; or represent C₁-C₈-halogenoalkyl, C₃-C₈-halogenocycloalkyl having in each case 1 to 9 fluorine-, chlorine- and/or bromine atoms; or R⁸ and R⁹ together with the nitrogen atom to which they are attached, represent a saturated 5- to 8-membered heterocycle, wherein the heterocycle optionally has 1 or 2 additional, non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R¹⁰ represents hydrogen, C₁-C₈-alkyl, C₁-C₈-alkoxy, C₁-C₄-alkoxy-C₁-C₄-alkyl, or C₃-C₈-cycloalkyl; or represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms,
R¹¹ represents hydrogen or C₁-C₆-alkyl, and
A represents
(1) a radical of formula (A1) 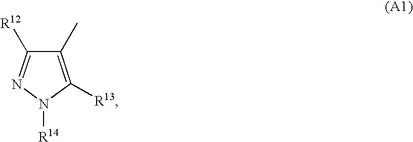
wherein
R¹² represents hydrogen, cyano, halogen, nitro, C₁-C₄-alkyl, C₁-C₄-alkoxy, C₁-C₄-alkylthio, or C₃-C₆-cycloalkyl; represents C₁-C₄-halogenoalkyl, C₁-C₄-halogenoalkoxy, or C₁ -C₄-halogenoalkylthio each having 1 to 5 halogen atoms; or represents aminocarbonyl or aminocarbonyl-C₁-C₄-alkyl,
R¹³ represents hydrogen, halogen, cyano, C₁-C₄-alkyl, C₁-C₄-alkoxy, or C₁-C₄-alkylthio, and
R¹⁴ represents hydrogen, C₁-C₄-alkyl, hydroxy-C₁-C₄-alkyl, C₂-C₆-alkenyl, C₃-C₆-cycloalkyl, C₁-C₄-alkylthio-C₁-C₄-alkyl, or C₁-C₄-alkoxy-C₁-C₄-alkyl; represents C₁-C₄-halogenoalkyl, C₁-C₄-halogenoalkylthio-C₁-C₄-alkyl, or C₁-C₄-halogenoalkoxy-C₁-C₄-alkyl each having 1 to 5 halogen atoms; or represents phenyl, or
(2) a radical of formula (A2) 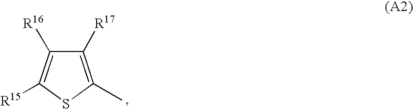
wherein
R¹⁵ and R¹⁶ independently of one another represent hydrogen, halogen, C₁-C₄-alkyl, or C₁-C₄-halogenoalkyl having 1 to 5 halogen atoms, and
R¹⁷ represents halogen, cyano or C₁-C₄-alkyl; or represents C₁-C₄-halogenoalkyl or C₁-C₄-halogenoalkoxy each having 1 to 5 halogen atoms, or
(3) a radical of formula (A3) 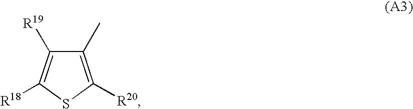
wherein
R¹⁸ and R¹⁹ independently of one another represent hydrogen, halogen, C₁-C₄-alkyl, or C₁-C₄-halogenoalkyl having 1 to 5 halogen atoms, and
R²⁰ represents hydrogen, halogen, C₁-C₄-alkyl, or C₁-C₄-halogenoalkyl having 1 to 5 halogen atoms, or
(4) a radical of formula (A4) 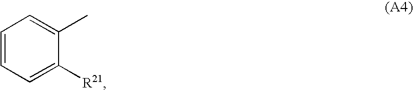
wherein R²¹ represents hydrogen, halogen, hydroxyl, cyano, or C₁-C₆-alkyl; or represents C₁-C₄-halogenoalkyl, C₁-C₄-halogenoalkoxy, or C₁-C₄-halogenoalkylthio each having 1 to 5 halogen atoms, or
(5) a radical of formula (A5) 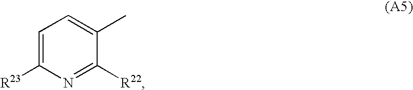
wherein
R²² represents halogen, hydroxyl, cyano, C₁-C₄-alkyl, C₁-C₄-alkoxy, or C₁-C₄-alkylthio; or represents C₁-C₄-halogenoalkyl, C₁-C₄-halogenoalkylthio, or C₁-C₄-halogenoalkoxy each having 1 to 5 halogen atoms, and
R²³ represents hydrogen, halogen, cyano, C₁-C₄-alkyl, C₁-C₄-alkoxy, or C₁-C₄-alkylthio; represents C₁-C₄-halogenoalkyl or C₁-C₄-halogenoalkoxy each having 1 to 5 halogen atoms; or represents C₁-C₄-alkylsulphinyl or C₁-C₄-alkylsulphonyl, or
(6) a radical of formula (A6) 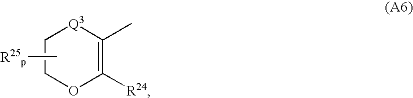
wherein
R²⁴ represents C₁-C₄-alkyl or C₁-C₄-halogenoalkyl having 1 to 5 halogen atoms,
R²⁵ represents C₁-C₄-alkyl,
Q³ represents a sulphur or oxygen atom, SO, SO₂, or CH₂, and
p represents 0, 1, or 2, with the proviso that R²⁵ represents identical or different radicals if p represents 2, or
(7) a radical of formula (A7) 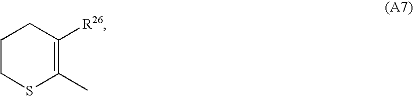
wherein R²⁶ represents C₁-C₄-alkyl or C₁-C₄-halogenoalkyl having 1 to 5 halogen atoms, or
(8) a radical of formula (A8) 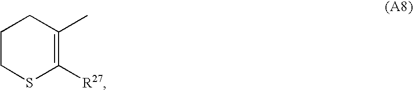
wherein R²⁷ represents C₁-C₄-alkyl or C₁-C₄-halogenoalkyl having 1 to 5 halogen atoms, or
(9) a radical of formula (A9) 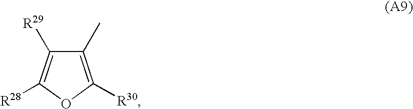
wherein
R²⁸ and R²⁹ independently of one another represent hydrogen, halogen, amino, or C₁-C₄-alkyl; or represent C₁-C₄-halogenoalkyl having 1 to 5 halogen atoms, and
R³⁰ represents hydrogen, halogen, C₁-C₄-alkyl, or C₁-C₄-halogenoalkyl having 1 to 5 halogen atoms, or
(10) a radical of formula (A10) 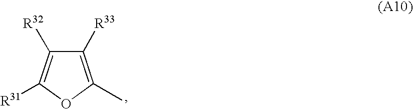
wherein
R³¹ and R³² independently of one another represent hydrogen, halogen, amino, nitro, C₁-C₄-alkyl, or C₁-C₄-halogenoalkyl having 1 to 5 halogen atoms, and
R³³ represents hydrogen, halogen, C₁-C₄-alkyl, or C₁-C₄-halogenoalkyl having 1 to 5 halogen atoms, or
(11) a radical of formula (A11) 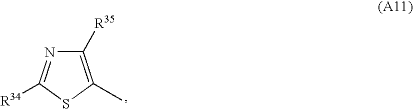
wherein
R³⁴ represents hydrogen, halogen, amino, C₁-C₄-alkylamino, di(C₁-C₄-alkyl)amino, cyano, C₁-C₄-alkyl, or C₁-C₄-halogenoalkyl having 1 to 5 halogen atoms, and
R³⁵ represents halogen, C₁-C₄-alkyl, or C₁-C₄-halogenoalkyl having 1 to 5 halogen atoms, or
(12) a radical of formula (A12) 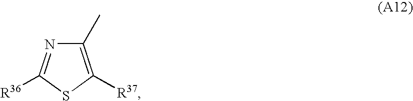
wherein
R³⁶ represents hydrogen, halogen, amino, C₁-C₄-alkylamino, di(C₁-C₄-alkyl)amino, cyano, C₁-C₄-alkyl, or C₁-C₄-halogenoalkyl having 1 to 5 halogen atoms, and
R³⁷ represents halogen, C₁-C₄-alkyl, or C₁-C₄-halogenoalkyl having 1 to 5 halogen atoms, or
(13) a radical of formula (A13) 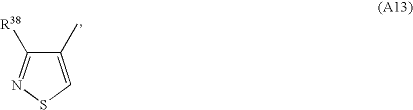
wherein R³⁸ represents halogen, C₁-C₄-alkyl, or C₁-C₄-halogenoalkyl having 1 to 5 halogen atoms, or
(14) a radical of formula (A14) 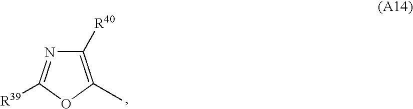
wherein
R³⁹ represents hydrogen or C₁-C₄-alkyl, and
R⁴⁰ represents halogen or C₁-C₄-alkyl, or
(15) a radical of formula (A15) 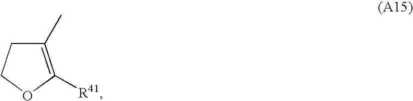
wherein R⁴¹ represents C₁-C₄-alkyl or C₁-C₄-halogenoalkyl having 1 to 5 halogen atoms, or
(16) a radical of formula (A16) 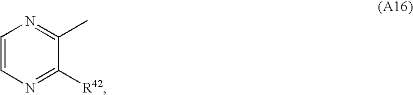
wherein R⁴² represents hydrogen, halogen, C₁-C₄-alkyl, or C₁-C₄-halogenoalkyl having 1 to 5 halogen atoms, or
(17) a radical of formula (A17) 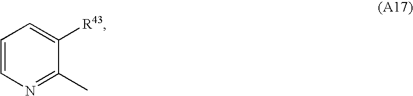
wherein R⁴³ represents halogen, hydroxyl, C₁-C₄-alkyl, C₁-C₄-alkoxy, or C₁-C₄-alkylthio, or represents C₁-C₄-halogenoalkyl, C₁-C₄-halogenoalkylthio, or C₁-C₄-halogenoalkoxy each having 1 to 5 halogen atoms,
with the exception of pyridinylanilides of formula (I-15) in which
R represents hydrogen,
R^{1a} and R^{2a} independently of one another each represents halogen; straight-chain or branched alkyl having 1 to 4 carbon atoms; or straight-chain or branched halogenoalkyl having 1 to 4 carbon atoms,
R⁴ represents hydrogen, and
A represents
(i) a radical of formula (A1) 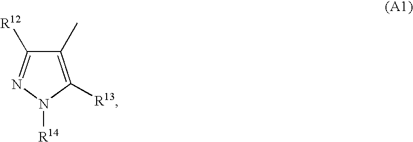
wherein
R¹² represents halogen, C₁-C₄-alkyl, or C₁-C₄-halogenoalkyl,
R¹³ represents hydrogen, and
R¹⁴ represents methyl, or
(ii) a radical of formula (A2) 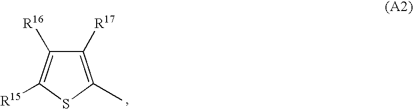
wherein
R¹⁵ and R¹⁶ independently of one another represent hydrogen or C₁-C₄-alkyl, and
R¹⁷ represents halogen, C₁-C₄-alkyl, or C₁-C₄-halogenoalkyl, or
(iii) a radical of formula (A4) 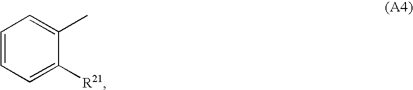
wherein R²¹ represents halogen, C₁-C₄-alkyl, or C₁-C₄-halogenoalkyl, or
(iv) a radical of formula (A5) 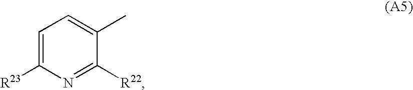
wherein
R²² represents halogen, and
R²³ represents hydrogen, or
(v) a radical of formula (A6) 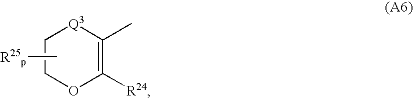
wherein
R²⁴ represents methyl,
Q³ represents a sulphur atom or CH₂, and
p represents 0, or
(vi) a radical of formula (A9) 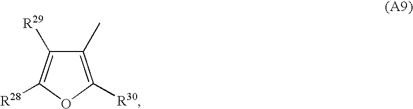
wherein
R²⁸ and R²⁹ independently of one another each represent hydrogen or C₁-C₄-alkyl, and
R³⁰ represents methyl, or
(vii) a radical of formula (A11) 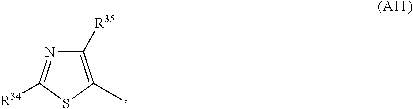
wherein
R³⁴ represents hydrogen or C₁-C₄-alkyl, and
R³⁵ represents halogen, C₁-C₄-alkyl, or C₁-C₄-halogenoalkyl, or
(viii) a radical of formula (A16) 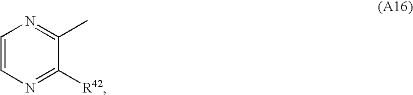
wherein R⁴² represents halogen.

31. A pyridinylanilide of formula (I-16) 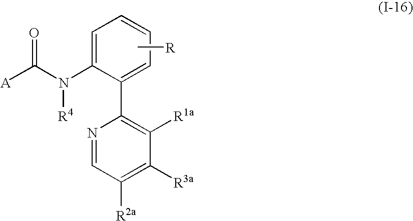
in which
R represents hydrogen, fluorine, chlorine, methyl, or trifluoromethyl;
R^{1a}, R^{2a}, and R^{3a} independently of one another each represents halogen, cyano, nitro, amino, hydroxyl, formyl, carboxyl, carbamoyl, or thiocarbamoyl; represent straight-chain or branched alkyl, hydroxyalkyl, oxoalkyl, alkoxy, alkoxyalkyl, alkylthioalkyl, dialkoxyalkyl, alkylthio, alkylsulfinyl, or alkylsulfonyl having in each case 1 to 8 carbon atoms in the respective alkyl moiety; represent straight-chain or branched alkenyl or alkenyloxy having in each case 2 to 6 carbon atoms; represent straight-chain or branched halogenoalkyl, halogenoalkoxy, halogenoalkylthio, halogenoalkylsulfinyl, or halogenoalkylsulfonyl having in each case 1 to 6 carbon atoms and 1 to 13 identical or different halogen atoms; represent straight-chain or branched halogenoalkenyl or halogenoalkenyloxy having in each case 2 to 6 carbon atoms and 1 to 11 identical or different halogen atoms; represent straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, or dialkylaminocarbonyloxy having 1 to 6 carbon atoms in the respective hydrocarbon chain; represent alkenylcarbonyl or alkynylcarbonyl having 2 to 6 carbon atoms in the respective hydrocarbon chain; represent cycloalkyl or cycloalkyloxy having in each case 3 to 6 carbon atoms; represent the group —C(Q¹)=N-Q², wherein
Q¹ represents hydrogen, hydroxyl, C₁-C₄-alkyl, C₁-C₄-halogenoalkyl having 1 to 9 identical or different halogen atoms, or C₃-C₆-cycloalkyl, and
Q² represents hydroxyl, amino, methylamino, phenyl, or benzyl; or represents C₁-C₄-alkyl or C₁-C₄-alkoxy, each of which is optionally substituted by halogen, cyano, hydroxyl, C₁-C₄-alkoxy, C₁-C₄-alkylthio, C₁-C₄-alkylamino, di(C₁-C₄-alkyl)amino, or phenyl; or represents C₂-C₄-alkenyloxy or C₂-C₄-alkynyloxy;
represent phenyl, phenoxy, phenylthio, benzoyl, benzoylethenyl, cinnamoyl, or heterocyclyl; or represent phenylalkyl, phenylalkyloxy, phenylalkylthio, or heterocyclylalkyl having in each case 1 to 3 carbon atoms in the respective alkyl moieties, each of which is optionally mono- to tri-substituted, identically or differently, in the ring moiety by halogen or straight-chain or branched C₁-C₄-alkyl or C₁-C₄-alkoxy;
R⁴ represents hydrogen, C₁-C₈-alkyl, C₁-C₆-alkylsulfinyl, C₁-C₆-alkylsulfonyl, C₁-C₄-alkoxy-C₁-C₄-alkyl, or C₃-C₈-cycloalkyl; represents C₁-C₆-halogenoalkyl, C₁-C₄-halogenoalkylthio, C₁-C₄-halogenoalkylsulfinyl, C₁-C₄-halogenoalkylsulfonyl, halogeno-C₁-C₄-alkoxy-C₁-C₄-alkyl, or C₃-C₈-halogenocycloalkyl having in each case 1 to 9 fluorine-, chlorine-, and/or bromine atoms;
represents formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C3-halogenoalkyl)carbonyl-C₁-C₃-alkyl, or (C₁-C₃-halogenoalkoxy)carbonyl-C₁-C₃-alkyl having in each case 1 to 7 fluorine-, chlorine-, and/or bromine atoms; represents (C₁-C₃-alkyl)carbonyl-C₁-C₃-halogenoalkyl or (C₁-C₃-alkoxy)carbonyl-C₁-C₃-halogenoalkyl having in each case 1 to 6 fluorine-, chlorine- ,and/or bromine atoms represents (C₁-C₃-halogenoalkyl)carbonyl-C₁-C₃-halogenoalkyl, (C₁-C₃-halogenoalkoxy)-carbonyl-C₁-C₃-halogenoalkyl having in each case 1 to 13 fluorine-, chlorine-, and/or bromine atoms; or represents —COR⁵, —CONR⁶R⁷, or —CH₂NR⁸R⁹,
R⁵ represents hydrogen, C₁-C₈-alkyl, C₁-C₈-alkoxy, C₁-C₄-alkoxy-C₁-C₄-alkyl, or C₃-C₈-cycloalkyl; represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms; or represents —COR¹⁰,
R⁶ and R⁷ independently of one another represent hydrogen, C₁-C₈-alkyl, C₁-C₄-alkoxy-C₁-C₄-alkyl, or C₃-C₈-cycloalkyl; or represent C₁-C₈-halogenoalkyl, halogeno-C₁-C₄-alkoxy-C₁-C₄-alkyl, or C₃-C₈-halogenocycloalkyl having in each case 1 to 9 fluorine-, chlorine-, and/or bromine atoms; or R⁶ and R⁷ together with the nitrogen atom to which they are attached represent a saturated 5- to 8-membered heterocycle, wherein the heterocycle optionally has 1 or 2 additional non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R⁸ and R⁹ independently of one another represent hydrogen, C₁-C₈-alkyl, or C₃-C₈-cycloalkyl; or represent C₁-C₈-halogenoalkyl, C₃-C₈-halogenocycloalkyl having in each case 1 to 9 fluorine-, chlorine- and/or bromine atoms; or R⁸ and R⁹ together with the nitrogen atom to which they are attached, represent a saturated 5- to 8-membered heterocycle, wherein the heterocycle optionally has 1 or 2 additional, non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R¹⁰ represents hydrogen, C₁-C₈-alkyl, C₁-C₈-alkoxy, C₁-C₄-alkoxy-C₁-C₄-alkyl, or C₃-C₈-cycloalkyl; or represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms,
R¹¹ represents hydrogen or C₁-C₆-alkyl, and
A represents
(1) a radical of formula (A1) 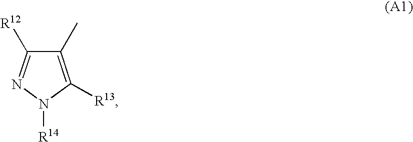
wherein
R¹² represents hydrogen, cyano, halogen, nitro, C₁-C₄-alkyl, C₁-C₄-alkoxy, C₁-C₄-alkylthio, or C₃-C₆-cycloalkyl; represents C₁-C₄-halogenoalkyl, C₁-C₄-halogenoalkoxy, or C₁-C₄-halogenoalkylthio each having 1 to 5 halogen atoms; or represents aminocarbonyl or aminocarbonyl-C₁-C₄-alkyl,
R¹³ represents hydrogen, halogen, cyano, C₁-C₄-alkyl, C₁-C₄-alkoxy, or C₁-C₄-alkylthio, and
R¹⁴ represents hydrogen, C₁-C₄-alkyl, hydroxy-C₁-C₄-alkyl, C₂-C₆-alkenyl, C₃-C₆-cycloalkyl, C₁-C₄-alkylthio-C₁-C₄-alkyl, or C₁-C₄-alkoxy-C₁-C₄-alkyl; represents C₁-C₄-halogenoalkyl, C₁-C₄-halogenoalkylthio-C₁-C₄-alkyl, or C₁-C₄-halogenoalkoxy-C₁-C₄-alkyl each having 1 to 5 halogen atoms; or represents phenyl, or
(2) a radical of formula (A2) 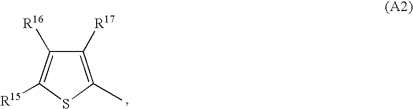
wherein
R¹⁵ and R¹⁶ independently of one another represent hydrogen, halogen, C₁-C₄-alkyl, or C₁-C₄-halogenoalkyl having 1 to 5 halogen atoms, and
R¹⁷ represents halogen, cyano or C₁-C₄-alkyl; or represents C₁-C₄-halogenoalkyl or C₁-C₄-halogenoalkoxy each having 1 to 5 halogen atoms, or
(3) a radical of formula (A3) 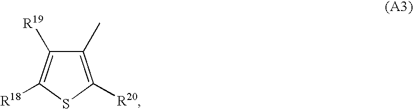
wherein
R¹⁸ and R¹⁹ independently of one another represent hydrogen, halogen, C₁-C₄-alkyl, or C₁-C₄-halogenoalkyl having 1 to 5 halogen atoms, and
R²⁰ represents hydrogen, halogen, C₁-C₄-alkyl, or C₁-C₄-halogenoalkyl having 1 to 5 halogen atoms, or
(4) a radical of formula (A4) 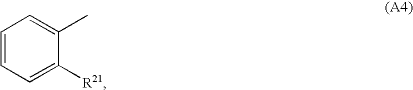
wherein R²¹ represents hydrogen, halogen, hydroxyl, cyano, or C₁-C₆-alkyl; or represents C₁-C₄-halogenoalkyl, C₁-C₄-halogenoalkoxy, or C₁-C₄-halogenoalkylthio each having 1 to 5 halogen atoms, or
(5) a radical of formula (A5) 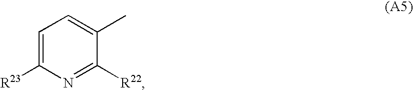
wherein
R²² represents halogen, hydroxyl, cyano, C₁-C₄-alkyl, C₁-C₄-alkoxy, or C₁-C₄-alkylthio; or represents C₁-C₄-halogenoalkyl, C₁-C₄-halogenoalkylthio, or C₁-C₄-halogenoalkoxy each having 1 to 5 halogen atoms, and
R²³ represents hydrogen, halogen, cyano, C₁-C₄-alkyl, C₁-C₄-alkoxy, or C₁-C₄-alkylthio; represents C₁-C₄-halogenoalkyl or C₁-C₄-halogenoalkoxy each having 1 to 5 halogen atoms; or represents C₁-C₄-alkylsulphinyl or C₁-C₄-alkylsulphonyl, or
(6) a radical of formula (A6) 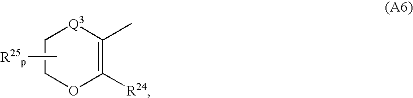
wherein
R²⁴ represents C₁-C₄-alkyl or C₁-C₄-halogenoalkyl having 1 to 5 halogen atoms,
R²⁵ represents C₁-C₄-alkyl,
Q³ represents a sulphur or oxygen atom, SO, SO₂, or CH₂, and
p represents 0, 1, or 2, with the proviso that R²⁵ represents identical or different radicals if p represents 2, or
(7) a radical of formula (A7) 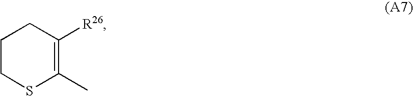
wherein R²⁶ represents C₁-C₄-alkyl or C₁-C₄-halogenoalkyl having 1 to 5 halogen atoms, or
(8) a radical of formula (A8) 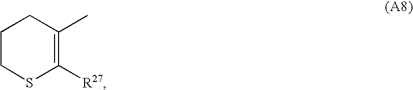
wherein R²⁷ represents C₁-C₄-alkyl or C₁-C₄-halogenoalkyl having 1 to 5 halogen atoms, or
(9) a radical of formula (A9) 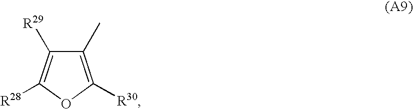
wherein
R²⁸ and R²⁹ independently of one another represent hydrogen, halogen, amino, or C₁-C₄-alkyl; or represent C₁-C₄-halogenoalkyl having 1 to 5 halogen atoms, and
R³⁰ represents hydrogen, halogen, C₁-C₄-alkyl, or C₁-C₄-halogenoalkyl having 1 to 5 halogen atoms, or
(10) a radical of formula (A10) 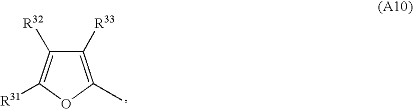
wherein
R³¹ and R³² independently of one another represent hydrogen, halogen, amino, nitro, C₁-C₄-alkyl, or C₁-C₄-halogenoalkyl having 1 to 5 halogen atoms, and
R³³ represents hydrogen, halogen, C₁-C₄-alkyl, or C₁-C₄-halogenoalkyl having 1 to 5 halogen atoms, or
(11) a radical of formula (A11) 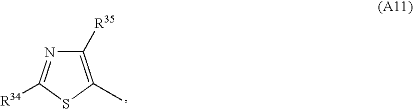
wherein
R³⁴ represents hydrogen, halogen, amino, C₁-C₄-alkylamino, di(C₁-C₄-alkyl)amino, cyano, C₁-C₄-alkyl, or C₁-C₄-halogenoalkyl having 1 to 5 halogen atoms, and
R³⁵ represents halogen, C₁-C₄-alkyl, or C₁-C₄-halogenoalkyl having 1 to 5 halogen atoms, or
(12) a radical of formula (A12) 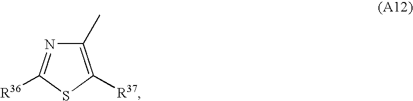
wherein
R³⁶ represents hydrogen, halogen, amino, C₁-C₄-alkylamino, di(C₁-C₄-alkyl)amino, cyano, C₁-C₄-alkyl, or C₁-C₄-halogenoalkyl having 1 to 5 halogen atoms, and
R³⁷ represents halogen, C₁-C₄-alkyl, or C₁-C₄-halogenoalkyl having 1 to 5 halogen atoms, or
(13) a radical of formula (A13) 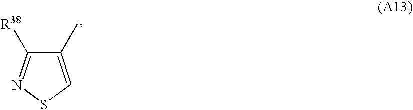
wherein R³⁸ represents halogen, C₁-C₄-alkyl, or C₁-C₄-halogenoalkyl having 1 to 5 halogen atoms, or
(14) a radical of formula (A14) 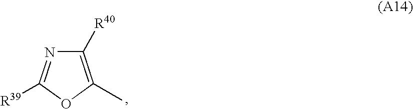
wherein
R³⁹ represents hydrogen or C₁-C₄-alkyl, and
R⁴⁰ represents halogen or C₁-C₄-alkyl, or
(15) a radical of formula (A15) 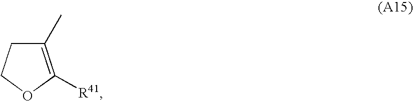
wherein R⁴¹ represents C₁-C₄-alkyl or C₁-C₄-halogenoalkyl having 1 to 5 halogen atoms, or
(16) a radical of formula (A16) 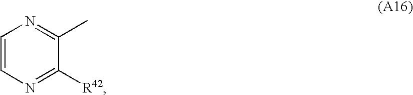
wherein R⁴² represents hydrogen, halogen, C₁-C₄-alkyl, or C₁-C₄-halogenoalkyl having 1 to 5 halogen atoms, or
(17) a radical of formula (A17) 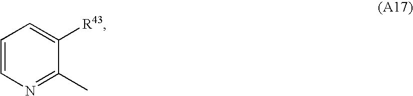
wherein R⁴³ represents halogen, hydroxyl, C₁-C₄-alkyl, C₁-C₄-alkoxy, or C₁-C₄-alkylthio, or represents C₁-C₄-halogenoalkyl, C₁-C₄-halogenoalkylthio, or C₁-C₄-halogenoalkoxy each having 1 to 5 halogen atoms,
with the exception of pyridinylanilides of formula (I-16) in which
R represents hydrogen,
R^{1a}, R^{2a}, and R^{3a} independently of one another each represents halogen; straight-chain or branched alkyl having 1 to 4 carbon atoms; or straight-chain or branched halogenoalkyl having 1 to 4 carbon atoms,
R⁴ represents hydrogen, and
A represents
(i) a radical of formula (A1) 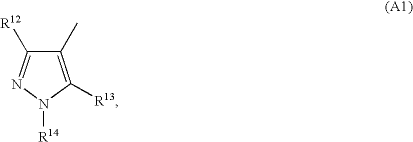
wherein
R¹² represents halogen, C₁-C₄-alkyl, or C₁-C₄-halogenoalkyl,
R¹³ represents hydrogen, and
R¹⁴ represents methyl, or
(ii) a radical of formula (A2) 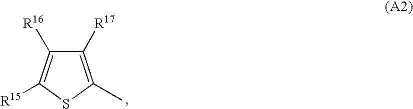
wherein
R¹⁵ and R¹⁶ independently of one another represent hydrogen or C₁-C₄-alkyl, and
R¹⁷ represents halogen, C₁-C₄-alkyl, or C₁-C₄-halogenoalkyl, or
(iii) a radical of formula (A4) 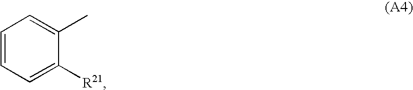
wherein R²¹ represents halogen, C₁-C₄-alkyl, or C₁-C₄-halogenoalkyl, or
(iv) a radical of formula (A5) 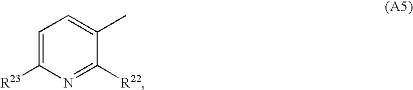
wherein
R²² represents halogen, and
R²³ represents hydrogen, or
(v) a radical of formula (A6) 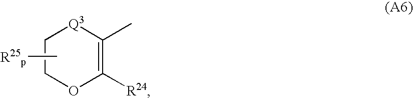
wherein
R²⁴ represents methyl,
Q³ represents a sulphur atom or CH₂, and
p represents 0, or
(vi) a radical of formula (A9) 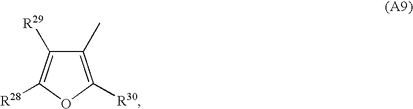
wherein
R²⁸ and R²⁹ independently of one another each represent hydrogen or C₁-C₄-alkyl, and
R³⁰ represents methyl, or
(vii) a radical of formula (A11) 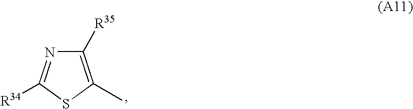
wherein
R³⁴ represents hydrogen or C₁-C₄-alkyl, and
R³⁵ represents halogen, C₁-C₄-alkyl, or C₁-C₄-halogenoalkyl, or
(viii) a radical of formula (A16) 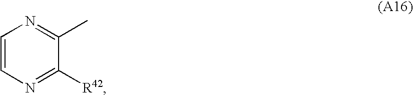
wherein R⁴² represents halogen.

32. A process for preparing pyridinylanilides of formula (I) according to claim 22 comprising
(a) reacting a carboxylic acid derivative of formula (II) 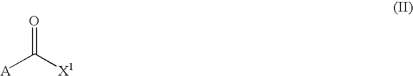
in which
X¹ represents halogen or hydroxyl, and
A is as defined for formula (I) in claim 22,
with an amine of formula (III) 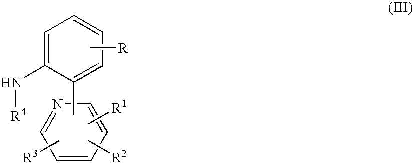
in which R, R¹, R², R³, and R⁴ are as defined for formula (I) in claim 22, optionally in the presence of a catalyst, optionally in the presence of a condensing agent, optionally in the presence of an acid binder, and optionally in the presence of a diluent,
or
(b) reacting a halogeno-carboxamide of formula (IV) 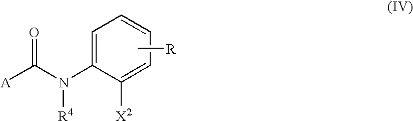
in which
R, R⁴, and A are as defined for formula (I) in claim 22, and
X² represents bromine or iodine,
with a boronic acid derivative of formula (V) 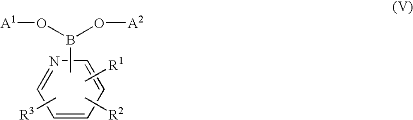
in which
R¹, R², and R³ are as defined for formula (I) in claim 22, and
A¹ and A² each represent hydrogen or A¹ and A² together represent tetramethylethylene,
in the presence of a catalyst, optionally in the presence of an acid binder, and optionally in the presence of a diluent,
or
(c) reacting a carboxamide boronic acid derivative of formula (VI) 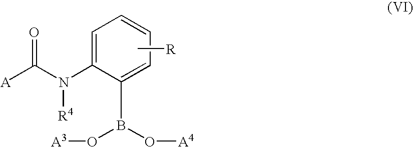
in which
R, R⁴, and A are as defined for formula (I) in claim 22, and
A³ and A⁴ each represent hydrogen or A³ and A⁴ together represent tetramethylethylene,
with a pyridinyl derivative of formula (VII) 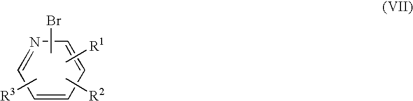
in which R¹, R², and R³ are as defined for formula (I) in claim 22, in the presence of a catalyst, optionally in the presence of an acid binder, and optionally in the presence of a diluent,
or
(d) reacting a halogeno-carboxamide of formula (IV) 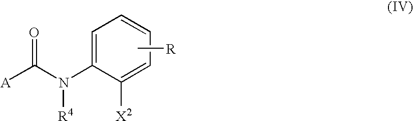
in which
R, R⁴, and A are as defined for formula (I) in claim 22, and
X² represents bromine or iodine,
with a pyridinyl derivative of formula (VII) 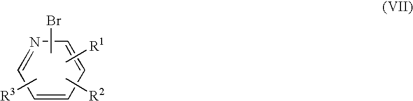
in which R¹, R², and R³ are as defined for formula (I) in claim 22, in the presence of a palladium or platinum catalyst and in the presence of 4,4,4′,4′,5,5,5′,5′-octamethyl-2,2′-bis-1,3,2-dioxaborolane[bis(pinacolato)-diboron], optionally in the presence of an acid binder, and optionally in the presence of a diluent,
or
(e) reacting a pyridinylanilide of formula (I-1) 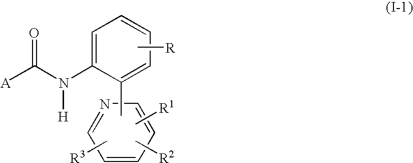
in which R, R¹, R², R³, and A are as defined for formula (I) in claim 22, with a halogenide of formula (VIII)
R^{4a}—X³ (VIII)
in which
X³ represents chlorine, bromine, or iodine,
R^{4a} represents C₁-C₈-alkyl, C₁-C₆-alkylsulfinyl, C₁-C₆-alkylsulfonyl, C₁-C₄-alkoxy-C₁-C₄-alkyl, or C₃-C₈-cycloalkyl; represents C₁-C₆-halogenoalkyl, C₁-C₄-halogenoalkylthio, C₁-C₄-halogenoalkylsulfinyl, C₁-C₄-halogenoalkylsulfonyl, halogeno-C₁-C₄-alkoxy-C₁-C₄-alkyl, or C₃-C₈-halogenocycloalkyl having in each case 1 to 9 fluorine-, chlorine- and/or bromine atoms; represents formyl-C₁-C₃-alkyl, (C₁-C₃-alkyl )carbonyl-C₁-C₃-alkyl, or (C₁-C₃-alkoxy)-carbonyl-C₁-C₃-alkyl; represents (C₁-C₃-halogenoalkyl)carbonyl-C₁-C₃-alkyl or (C₁-C₃-halogenoalkoxy)carbonyl-C₁-C₃-alkyl having in each case 1 to 7 fluorine-, chlorine-, and/or bromine atoms; represents (C₁-C₃-alkyl)carbonyl-C₁-C₃-halogenoalkyl or (C₁-C₃-alkoxy)carbonyl-C₁-C₃-halogenoalkyl having in each case 1 to 6 fluorine-, chlorine-, and/or bromine atoms; represents (C₁-C₃-halogenoalkyl)carbonyl-C₁-C₃-halogenoalkyl or (C₁-C₃-halogenoalkoxy)carbonyl-C₁-C₃-halogenoalkyl having in each case 1 to 13 fluorine-, chlorine-, and/or bromine atoms; or represents —COR⁵, —CONR⁶R⁷, or —CH₂NR⁸R⁹; and
R⁵, R⁶, R⁷, R⁸, and R⁹ are as defined for formula (I) in claim 22, in the presence of a base and in the presence of a diluent.

33. A composition for controlling unwanted microorganisms comprising one or more pyridinylanilides of formula (I) according to claim 22 and one or more extenders and/or surfactants.

34. A method for controlling unwanted microorganisms comprising applying an effective amount of one or more pyridinylanilides of formula (I) according to claim 22 to the microorganisms and/or their habitats.

35. A process for preparing compositions for controlling unwanted microorganisms comprising mixing one or more pyridinylanilides of formula (I) according to claim 22 with one or more extenders and/or surfactants.

36. An amine of formula (III) 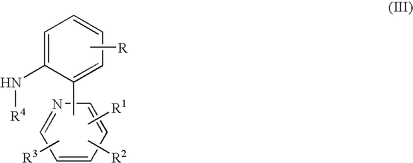
in which
R represents hydrogen, fluorine, chlorine, methyl, or trifluoromethyl;
R¹, R², and R³ independently of one another represent hydrogen, halogen, cyano, nitro, amino, hydroxyl, formyl, carboxyl, carbamoyl, or thiocarbamoyl; represent straight-chain or branched alkyl, hydroxyalkyl, oxoalkyl, alkoxy, alkoxyalkyl, alkylthioalkyl, dialkoxyalkyl, alkylthio, alkylsulfinyl, or alkylsulfonyl having in each case 1 to 8 carbon atoms in the respective alkyl moiety; represent straight-chain or branched alkenyl or alkenyloxy having in each case 2 to 6 carbon atoms; represent straight-chain or branched halogenoalkyl, halogenoalkoxy, halogenoalkylthio, halogenoalkylsulfinyl, or halogenoalkylsulfonyl having in each case 1 to 6 carbon atoms and 1 to 13 identical or different halogen atoms; represent straight-chain or branched halogenoalkenyl or halogenoalkenyloxy having in each case 2 to 6 carbon atoms and 1 to 11 identical or different halogen atoms; represent straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, or dialkylaminocarbonyloxy having 1 to 6 carbon atoms in the respective hydrocarbon chain; represent alkenylcarbonyl or alkynylcarbonyl having 2 to 6 carbon atoms in the respective hydrocarbon chain; represent cycloalkyl or cycloalkyloxy having in each case 3 to 6 carbon atoms; represent the group —C(Q¹)=N-Q², wherein
Q¹ represents hydrogen, hydroxyl, C₁-C₄-alkyl, C₁-C₄-halogenoalkyl having 1 to 9 identical or different halogen atoms, or C₃-C₆-cycloalkyl and
Q² represents hydroxyl, amino, methylamino, phenyl, or benzyl; represents C₁-C₄-alkyl or C₁-C₄-alkoxy, each of which is optionally substituted by halogen, cyano, hydroxyl, C₁-C₄-alkoxy, C₁-C₄-alkylthio, C₁-C₄-alkylamino, di(C₁-C₄-alkyl)amino, or phenyl; or represents C₂-C₄-alkenyloxy or C₂-C₄-alkynyloxy;
represents phenyl, phenoxy, phenylthio, benzoyl, benzoylethenyl, cinnamoyl, or heterocyclyl, each of which is optionally mono- to tri-substituted, identically or differently, in the ring moiety by halogen or straight-chain or branched C₁-C₄-alkyl and C₁-C₄-alkoxy; or represents phenylalkyl, phenylalkyloxy, phenylalkylthio, or heterocyclylalkyl having in each case 1 to 3 carbon atoms in the respective alkyl moieties, each of which is optionally mono- to tri-substituted, identically or differently, in the ring moiety by halogen or straight-chain or branched C₁-C₄-alkyl and C₁-C₄-alkoxy;
or when R² and R³ are attached to the pyridinyl moiety in an ortho position to each other, then R¹ is defined as above and R² and R³ together further represent C₃-C₄-alkylene, C₃-C₄-alkenylene, C₂-C₃-oxyalkylene, or C₁-C₂-dioxyalkylene, each of which is optionally mono- to tetra-substituted, identically or differently, by fluorine, chlorine, oxo, methyl, ethyl, or trifluoromethyl;
R⁴ represents hydrogen, C₁-C₈-alkyl, C₁-C₆-alkylsulfinyl, C₁-C₆-alkylsulfonyl, C₁-C₄-alkoxy-C₁-C₄-alkyl, or C₃-C₈-cycloalkyl; represents C₁-C₆-halogenoalkyl, C₁-C₄-halogenoalkylthio, C₁-C₄-halogenoalkylsulfinyl, C₁-C₄-halogenoalkylsulfonyl, halogeno-C₁-C₄-alkoxy-C₁-C₄-alkyl, or C₃-C₈-halogenocycloalkyl having in each case 1 to 9 fluorine-, chlorine-, and/or bromine atoms; represents formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C₃-halogenoalkyl)carbonyl-C₁-C₃-alkyl, or (C₁-C₃-halogenoalkoxy)carbonyl-C₁-C₃-alkyl having in each case 1 to 7 fluorine-, chlorine-, and/or bromine atoms; represents (C₁-C₃-alkyl)carbonyl-C₁-C₃-halogenoalkyl or (C₁-C₃-alkoxy)carbonyl-C₁-C₃-halogenoalkyl having in each case 1 to 6 fluorine-, chlorine- ,and/or bromine atoms represents (C₁-C₃-halogenoalkyl)carbonyl-C₁-C₃-halogenoalkyl, (C₁-C₃-halogenoalkoxy)-carbonyl-C₁-C₃-halogenoalkyl having in each case 1 to 13 fluorine-, chlorine-, and/or bromine atoms; or represents —COR⁵, —CONR⁶R⁷, or —CH₂NR⁸R⁹,
R⁵ represents hydrogen, C₁-C₈-alkyl, C₁-C₈-alkoxy, C₁-C₄-alkoxy-C₁-C₄-alkyl, or C₃-C₈-cycloalkyl; represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms; or represents —COR¹⁰,
R⁶ and R⁷ independently of one another represent hydrogen, C₁-C₈-alkyl, C₁-C₄-alkoxy-C₁-C₄-alkyl, or C₃-C₈-cycloalkyl; or represent C₁-C₈-halogenoalkyl, halogeno-C₁-C₄-alkoxy-C₁-C₄-alkyl, or C₃-C₈-halogenocycloalkyl having in each case 1 bis 9 fluorine-, chlorine-, and/or bromine atoms; or R⁶ and R⁷ together with the nitrogen atom to which they are attached represent a saturated 5- to 8-membered heterocycle, wherein the heterocycle optionally has 1 or 2 additional non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R⁸ and R⁹ independently of one another represent hydrogen, C₁-C₈-alkyl, or C₃-C₈-cycloalkyl; or represent C₁-C₈-halogenoalkyl, C₃-C₈-halogenocycloalkyl having in each case 1 to 9 fluorine-, chlorine- and/or bromine atoms; or R⁸ and R⁹ together with the nitrogen atom to which they are attached, represent a saturated 5- to 8-membered heterocycle, wherein the heterocycle optionally has 1 or 2 additional, non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R¹⁰ represents hydrogen, C₁-C₈-alkyl, C₁-C₈-alkoxy, C₁-C₄-alkoxy-C₁-C₄-alkyl, or C₃-C₈-cycloalkyl; or represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms, and
R¹¹ represents hydrogen or C₁-C₆-alkyl,
with the exception of amines of formula (III) in which
R represents hydrogen, and
R¹, R², and R³ independently of one another represent hydrogen, halogen, straight-chain or branched alkyl having 1 to 4 carbon atoms, or straight-chain or branched halogenoalkyl having 1 to 4 carbon atoms; and
R⁴ represents hydrogen.

37. An amine of the formula 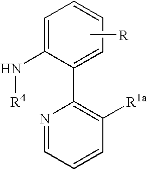
in which
R represents hydrogen, fluorine, chlorine, methyl, or trifluoromethyl;
R^{1a} represents halogen, cyano, nitro, amino, hydroxyl, formyl, carboxyl, carbamoyl, or thiocarbamoyl; represents straight-chain or branched alkyl, hydroxyalkyl, oxoalkyl, alkoxy, alkoxyalkyl, alkylthioalkyl, dialkoxyalkyl, alkylthio, alkylsulfinyl, or alkylsulfonyl having in each case 1 to 8 carbon atoms in the respective alkyl moiety; represents straight-chain or branched alkenyl or alkenyloxy having in each case 2 to 6 carbon atoms; represents straight-chain or branched halogenoalkyl, halogenoalkoxy, halogenoalkylthio, halogenoalkylsulfinyl, or halogenoalkylsulfonyl having in each case 1 to 6 carbon atoms and 1 to 13 identical or different halogen atoms; represents straight-chain or branched halogenoalkenyl or halogenoalkenyloxy having in each case 2 to 6 carbon atoms and 1 to 11 identical or different halogen atoms; represents straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, or dialkylaminocarbonyloxy having 1 to 6 carbon atoms in the respective hydrocarbon chain; represents alkenylcarbonyl or alkynylcarbonyl having 2 to 6 carbon atoms in the respective hydrocarbon chain; represents cycloalkyl or cycloalkyloxy having in each case 3 to 6 carbon atoms; represents the group —C(Q¹)=N-Q², wherein
Q¹ represents hydrogen, hydroxyl, C₁-C₄-alkyl, C₁-C₄-halogenoalkyl having 1 to 9 identical or different halogen atoms, or C₃-C₆-cycloalkyl and
Q² represents hydroxyl, amino, methylamino, phenyl, or benzyl; represents C₁-C₄-alkyl or C₁-C₄-alkoxy, each of which is optionally substituted by halogen, cyano, hydroxyl, C₁-C₄-alkoxy, C₁-C₄-alkylthio, C₁-C₄-alkylamino, di(C₁-C₄-alkyl)amino, or phenyl; or represents C₂-C₄-alkenyloxy or C₂-C₄-alkynyloxy;
represents phenyl, phenoxy, phenylthio, benzoyl, benzoylethenyl, cinnamoyl, or heterocyclyl; or represents phenylalkyl, phenylalkyloxy, phenylalkylthio, or heterocyclylalkyl having in each case 1 to 3 carbon atoms in the respective alkyl moieties, each of which is optionally mono- to tri-substituted, identically or differently, in the ring moiety by halogen or straight-chain or branched C₁-C₄-alkyl or C₁-C₄-alkoxy;
R⁴ represents hydrogen, C₁-C₈-alkyl, C₁-C₆-alkylsulfinyl, C₁-C₆-alkylsulfonyl, C₁-C₄-alkoxy-C₁-C₄-alkyl, or C₃-C₈-cycloalkyl; represents C₁-C₆-halogenoalkyl, C₁-C₄-halogenoalkylthio, C₁-C₄-halogenoalkylsulfinyl, C₁-C₄-halogenoalkylsulfonyl, halogeno-C₁-C₄-alkoxy-C₁-C₄-alkyl, or C₃-C₈-halogenocycloalkyl having in each case 1 to 9 fluorine-, chlorine-, and/or bromine atoms; represents formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C₃-halogenoalkyl)carbonyl-C₁-C₃-alkyl, or (C₁-C₃-halogenoalkoxy)carbonyl-C₁-C₃-alkyl having in each case 1 to 7 fluorine-, chlorine-, and/or bromine atoms; represents (C₁-C₃-alkyl)carbonyl-C₁-C₃-halogenoalkyl or (C₁-C₃-alkoxy)carbonyl-C₁-C₃-halogenoalkyl having in each case 1 to 6 fluorine-, chlorine- ,and/or bromine atoms represents (C₁-C₃-halogenoalkyl)carbonyl-C₁-C₃-halogenoalkyl, (C₁-C₃-halogenoalkoxy)-carbonyl-C₁-C₃-halogenoalkyl having in each case 1 to 13 fluorine-, chlorine-, and/or bromine atoms; or represents —COR⁵, —CONR⁶R⁷, or —CH₂NR⁸R⁹,
R⁵ represents hydrogen, C₁-C₈-alkyl, C₁-C₈-alkoxy, C₁-C₄-alkoxy-C₁-C₄-alkyl, or C₃-C₈-cycloalkyl; represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms; or represents —COR¹⁰,
R⁶ and R⁷ independently of one another represent hydrogen, C₁-C₈-alkyl, C₁-C₄-alkoxy-C₁-C₄-alkyl, or C₃-C₈-cycloalkyl; or represent C₁-C₈-halogenoalkyl, halogeno-C₁-C₄-alkoxy-C₁-C₄-alkyl, or C3-C₈-halogenocycloalkyl having in each case 1 bis 9 fluorine-, chlorine-, and/or bromine atoms; or R⁶ and R⁷ together with the nitrogen atom to which they are attached represent a saturated 5- to 8-membered heterocycle, wherein the heterocycle optionally has 1 or 2 additional non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R⁸ and R⁹ independently of one another represent hydrogen, C₁-C₈-alkyl, or C₃-C₈-cycloalkyl; or represent C₁-C₈-halogenoalkyl, C₃-C₈-halogenocycloalkyl having in each case 1 to 9 fluorine-, chlorine- and/or bromine atoms; or R⁸ and R⁹ together with the nitrogen atom to which they are attached, represent a saturated 5- to 8-membered heterocycle, wherein the heterocycle optionally has 1 or 2 additional, non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R¹⁰ represents hydrogen, C₁-C₈-alkyl, C₁-C₈-alkoxy, C₁-C₄-alkoxy-C₁-C₄-alkyl, or C₃-C₈-cycloalkyl; or represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms, and
R¹¹ represents hydrogen or C₁-C₆-alkyl.

38. An amine of the formula 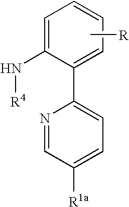
in which
R represents hydrogen, fluorine, chlorine, methyl, or trifluoromethyl;
R^{1a} represents halogen, cyano, nitro, amino, hydroxyl, formyl, carboxyl, carbamoyl, or thiocarbamoyl; represents straight-chain or branched alkyl, hydroxyalkyl, oxoalkyl, alkoxy, alkoxyalkyl, alkylthioalkyl, dialkoxyalkyl, alkylthio, alkylsulfinyl, or alkylsulfonyl having in each case 1 to 8 carbon atoms in the respective alkyl moiety; represents straight-chain or branched alkenyl or alkenyloxy having in each case 2 to 6 carbon atoms; represents straight-chain or branched halogenoalkyl, halogenoalkoxy, halogenoalkylthio, halogenoalkylsulfinyl, or halogenoalkylsulfonyl having in each case 1 to 6 carbon atoms and 1 to 13 identical or different halogen atoms; represents straight-chain or branched halogenoalkenyl or halogenoalkenyloxy having in each case 2 to 6 carbon atoms and 1 to 11 identical or different halogen atoms; represents straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, or dialkylaminocarbonyloxy having 1 to 6 carbon atoms in the respective hydrocarbon chain; represents alkenylcarbonyl or alkynylcarbonyl having 2 to 6 carbon atoms in the respective hydrocarbon chain; represents cycloalkyl or cycloalkyloxy having in each case 3 to 6 carbon atoms; represents the group —C(Q¹)=N-Q², wherein
Q¹ represents hydrogen, hydroxyl, C₁-C₄-alkyl, C₁-C₄-halogenoalkyl having 1 to 9 identical or different halogen atoms, or C₃-C₆-cycloalkyl, and
Q² represents hydroxyl, amino, methylamino, phenyl, or benzyl; or represents C₁-C₄-alkyl or C₁-C₄-alkoxy, each of which is optionally substituted by halogen, cyano, hydroxyl, C₁-C₄-alkoxy, C₁-C₄-alkylthio, C₁-C₄-alkylamino, di(C₁-C₄-alkyl)amino, or phenyl; or represents C₂-C₄-alkenyloxy or C₂-C₄-alkynyloxy;
represents phenyl, phenoxy, phenylthio, benzoyl, benzoylethenyl, cinnamoyl, or heterocyclyl; or represents phenylalkyl, phenylalkyloxy, phenylalkylthio, or heterocyclylalkyl having in each case 1 to 3 carbon atoms in the respective alkyl moieties, each of which is optionally mono- to tri-substituted, identically or differently, in the ring moiety by halogen or straight-chain or branched C₁-C₄-alkyl or C₁-C₄-alkoxy;
R⁴ represents hydrogen, C₁-C₈-alkyl, C₁-C₆-alkylsulfinyl, C₁-C₆-alkylsulfonyl, C₁-C₄-alkoxy-C₁-C₄-alkyl, or C₃-C₈-cycloalkyl; represents C₁-C₆-halogenoalkyl, C₁-C₄-halogenoalkylthio, C₁-C₄-halogenoalkylsulfinyl, C₁-C₄-halogenoalkylsulfonyl, halogeno-C₁-C₄-alkoxy-C₁-C₄-alkyl, or C₃-C₈-halogenocycloalkyl having in each case 1 to 9 fluorine-, chlorine-, and/or bromine atoms; represents formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C₃-halogenoalkyl)carbonyl-C₁-C₃-alkyl, or (C₁-C₃-halogenoalkoxy)carbonyl-C₁-C₃-alkyl having in each case 1 to 7 fluorine-, chlorine-, and/or bromine atoms; represents (C₁-C₃-alkyl)carbonyl-C₁-C₃-halogenoalkyl or (C₁-C₃-alkoxy)carbonyl-C₁-C₃-halogenoalkyl having in each case 1 to 6 fluorine-, chlorine- ,and/or bromine atoms represents (C₁-C₃-halogenoalkyl)carbonyl-C₁-C₃-halogenoalkyl, (C₁-C₃-halogenoalkoxy)-carbonyl-C₁-C₃-halogenoalkyl having in each case 1 to 13 fluorine-, chlorine-, and/or bromine atoms; or represents —COR⁵, —CONR⁶R⁷, or —CH₂NR⁸R⁹,
R⁵ represents hydrogen, C₁-C₈-alkyl, C₁-C₈-alkoxy, C₁-C₄-alkoxy-C₁-C₄-alkyl, or C₃-C₈-cycloalkyl; represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms; or represents —COR¹⁰,
R⁶ and R⁷ independently of one another represent hydrogen, C₁-C₈-alkyl, C₁-C₄-alkoxy-C₁-C₄-alkyl, or C₃-C₈-cycloalkyl; or represent C₁-C₈-halogenoalkyl, halogeno-C₁-C₄-alkoxy-C₁-C₄-alkyl, or C₃-C₈-halogenocycloalkyl having in each case 1 bis 9 fluorine-, chlorine-, and/or bromine atoms; or R⁶ and R⁷ together with the nitrogen atom to which they are attached represent a saturated 5- to 8-membered heterocycle, wherein the heterocycle optionally has 1 or 2 additional non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R⁸ and R⁹ independently of one another represent hydrogen, C₁-C₈-alkyl, or C₃-C₈-cycloalkyl; or represent C₁-C₈-halogenoalkyl, C₃-C₈-halogenocycloalkyl having in each case 1 to 9 fluorine-, chlorine- and/or bromine atoms; or R⁸ and R⁹ together with the nitrogen atom to which they are attached, represent a saturated 5- to 8-membered heterocycle, wherein the heterocycle optionally has 1 or 2 additional, non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R¹⁰ represents hydrogen, C₁-C₈-alkyl, C₁-C₈-alkoxy, C₁-C₄-alkoxy-C₁-C₄-alkyl, or C₃-C₈-cycloalkyl; or represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms, and
R¹¹ represents hydrogen or C₁-C₆-alkyl.

39. An amine of the formula 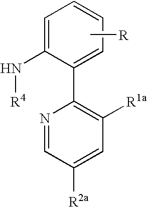
in which
R represents hydrogen, fluorine, chlorine, methyl, or trifluoromethyl;
R^{1a} and R^{2a} independently of one another represent halogen, cyano, nitro, amino, hydroxyl, formyl, carboxyl, carbamoyl, or thiocarbamoyl; represent straight-chain or branched alkyl, hydroxyalkyl, oxoalkyl, alkoxy, alkoxyalkyl, alkylthioalkyl, dialkoxyalkyl, alkylthio, alkylsulfinyl, or alkylsulfonyl having in each case 1 to 8 carbon atoms in the respective alkyl moiety; represent straight-chain or branched alkenyl or alkenyloxy having in each case 2 to 6 carbon atoms; represent straight-chain or branched halogenoalkyl, halogenoalkoxy, halogenoalkylthio, halogenoalkylsulfinyl, or halogenoalkylsulfonyl having in each case 1 to 6 carbon atoms and 1 to 13 identical or different halogen atoms; represent straight-chain or branched halogenoalkenyl or halogenoalkenyloxy having in each case 2 to 6 carbon atoms and 1 to 11 identical or different halogen atoms; represent straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, or dialkylaminocarbonyloxy having 1 to 6 carbon atoms in the respective hydrocarbon chain; represent alkenylcarbonyl or alkynylcarbonyl having 2 to 6 carbon atoms in the respective hydrocarbon chain; represent cycloalkyl or cycloalkyloxy having in each case 3 to 6 carbon atoms; represents the group —C(Q¹)=N-Q², wherein
Q¹ represents hydrogen, hydroxyl, C₁-C₄-alkyl, C₁-C₄-halogenoalkyl having 1 to 9 identical or different halogen atoms, or C₃-C₆-cycloalkyl, and
Q² represents hydroxyl, amino, methylamino, phenyl, or benzyl; or represents C₁-C₄-alkyl or C₁-C₄-alkoxy, each of which is optionally substituted by halogen, cyano, hydroxyl, C₁-C₄-alkoxy, C₁-C₄-alkylthio, C₁-C₄-alkylamino, di(C₁-C₄-alkyl)amino, or phenyl; or represents C₂-C₄-alkenyloxy or C₂-C₄-alkynyloxy,
represents phenyl, phenoxy, phenylthio, benzoyl, benzoylethenyl, cinnamoyl, or heterocyclyl; or represents phenylalkyl, phenylalkyloxy, phenylalkylthio, or heterocyclylalkyl having in each case 1 to 3 carbon atoms in the respective alkyl moieties, each of which is optionally mono- to tri-substituted, identically or differently, in the ring moiety by halogen or straight-chain or branched C₁-C₄-alkyl or C₁-C₄-alkoxy;
R⁴ represents hydrogen, C₁-C₈-alkyl, C₁-C₆-alkylsulfinyl, C₁-C₆-alkylsulfonyl, C₁-C₄-alkoxy-C₁-C₄-alkyl, or C₃-C₈-cycloalkyl; represents C₁-C₆-halogenoalkyl, C₁-C₄-halogenoalkylthio, C₁-C₄-halogenoalkylsulfinyl, C₁-C₄-halogenoalkylsulfonyl, halogeno-C₁-C₄-alkoxy-C₁-C₄-alkyl, or C₃-C₈-halogenocycloalkyl having in each case 1 to 9 fluorine-, chlorine-, and/or bromine atoms; represents formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C₃-halogenoalkyl)carbonyl-C₁-C₃-alkyl, or (C₁-C₃-halogenoalkoxy)carbonyl-C₁-C₃-alkyl having in each case 1 to 7 fluorine-, chlorine-, and/or bromine atoms; represents (C₁-C₃-alkyl)carbonyl-C₁-C₃-halogenoalkyl or (C₁-C₃-alkoxy)carbonyl-C₁-C₃-halogenoalkyl having in each case 1 to 6 fluorine-, chlorine- ,and/or bromine atoms represents (C₁-C₃-halogenoalkyl)carbonyl-C₁-C₃-halogenoalkyl, (C₁-C₃-halogenoalkoxy)-carbonyl-C₁-C₃-halogenoalkyl having in each case 1 to 13 fluorine-, chlorine-, and/or bromine atoms; or represents —COR⁵, —CONR⁶R⁷, or —CH₂NR⁸R⁹,
R⁵ represents hydrogen, C₁-C₈-alkyl, C₁-C₈-alkoxy, C₁-C₄-alkoxy-C₁-C₄-alkyl, or C₃-C₈-cycloalkyl; represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms; or represents —COR¹⁰,
R⁶ and R⁷ independently of one another represent hydrogen, C₁-C₈-alkyl, C₁-C₄-alkoxy-C₁-C₄-alkyl, or C₃-C₈-cycloalkyl; or represent C₁-C₈-halogenoalkyl, halogeno-C₁-C₄-alkoxy-C₁-C₄-alkyl, or C₃-C₈-halogenocycloalkyl having in each case 1 bis 9 fluorine-, chlorine-, and/or bromine atoms; or R⁶ and R⁷ together with the nitrogen atom to which they are attached represent a saturated 5- to 8-membered heterocycle, wherein the heterocycle optionally has 1 or 2 additional non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R⁸ and R⁹ independently of one another represent hydrogen, C₁-C₈-alkyl, or C₃-C₈-cycloalkyl; or represent C₁-C₈-halogenoalkyl, C₃-C₈-halogenocycloalkyl having in each case 1 to 9 fluorine-, chlorine- and/or bromine atoms; or R⁸ and R⁹ together with the nitrogen atom to which they are attached, represent a saturated 5- to 8-membered heterocycle, wherein the heterocycle optionally has 1 or 2 additional, non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R¹⁰ represents hydrogen, C₁-C₈-alkyl, C₁-C₈-alkoxy, C₁-C₄-alkoxy-C₁-C₄-alkyl, or C₃-C₈-cycloalkyl; or represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms, and
R¹¹ represents hydrogen or C₁-C₆-alkyl.

40. an amine of the formula 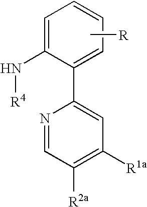
in which
R represents hydrogen, fluorine, chlorine, methyl, or trifluoromethyl;
R^{1a} and R^{2a} independently of one another represent halogen, cyano, nitro, amino, hydroxyl, formyl, carboxyl, carbamoyl, or thiocarbamoyl; represent straight-chain or branched alkyl, hydroxyalkyl, oxoalkyl, alkoxy, alkoxyalkyl, alkylthioalkyl, dialkoxyalkyl, alkylthio, alkylsulfinyl,or alkylsulfonyl having in each case 1 to 8 carbon atoms in the respective alkyl moiety; represent straight-chain or branched alkenyl or alkenyloxy having in each case 2 to 6 carbon atoms; represent straight-chain or branched halogenoalkyl, halogenoalkoxy, halogenoalkylthio, halogenoalkylsulfinyl, or halogenoalkylsulfonyl having in each case 1 to 6 carbon atoms and 1 to 13 identical or different halogen atoms; represent straight-chain or branched halogenoalkenyl or halogenoalkenyloxy having in each case 2 to 6 carbon atoms and 1 to 11 identical or different halogen atoms; represent straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, or dialkylaminocarbonyloxy having 1 to 6 carbon atoms in the respective hydrocarbon chain; represent alkenylcarbonyl or alkynylcarbonyl having 2 to 6 carbon atoms in the respective hydrocarbon chain; represent cycloalkyl or cycloalkyloxy having in each case 3 to 6 carbon atoms; or represent the group —C(Q¹)=N-Q², wherein
Q¹ represents hydrogen, hydroxyl, C₁-C₄-alkyl, C₁-C₄-halogenoalkyl having 1 to 9 identical or different halogen atoms, or C₃-C₆-cycloalkyl, and
Q² represents hydroxyl, amino, methylamino, phenyl, or benzyl; or represents C₁-C₄-alkyl or C₁-C₄-alkoxy, each of which is optionally substituted by halogen, cyano, hydroxyl, C₁-C₄-alkoxy, C₁-C₄-alkylthio, C₁-C₄-alkylamino, di(C₁-C₄-alkyl)amino, or phenyl; or represents C₂-C₄-alkenyloxy or C₂-C₄-alkynyloxy;
represent phenyl, phenoxy, phenylthio, benzoyl, benzoylethenyl, cinnamoyl, or heterocyclyl; or represent phenylalkyl, phenylalkyloxy, phenylalkylthio, or heterocyclylalkyl having in each case 1 to 3 carbon atoms in the respective alkyl moieties, each of which is optionally mono- to tri-substituted, identically or differently, in the ring moiety by halogen or straight-chain or branched C₁-C₄-alkyl or C₁-C₄-alkoxy;
R⁴ represents hydrogen, C₁-C₈-alkyl, C₁-C₆-alkylsulfinyl, C₁-C₆-alkylsulfonyl, C₁-C₄-alkoxy-C₁-C₄-alkyl, or C₃-C₈-cycloalkyl; represents C₁-C₆-halogenoalkyl, C₁-C₄-halogenoalkylthio, C₁-C₄-halogenoalkylsulfinyl, C₁-C₄-halogenoalkylsulfonyl, halogeno-C₁-C₄-alkoxy-C₁-C₄-alkyl, or C₃-C₈-halogenocycloalkyl having in each case 1 to 9 fluorine-, chlorine-, and/or bromine atoms; represents formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C₃-halogenoalkyl)carbonyl-C₁-C₃-alkyl, or (C₁-C₃-halogenoalkoxy)carbonyl-C₁-C₃-alkyl having in each case 1 to 7 fluorine-, chlorine-, and/or bromine atoms; represents (C₁-C₃-alkyl)carbonyl-C₁-C₃-halogenoalkyl or (C₁-C₃-alkoxy)carbonyl-C₁-C₃-halogenoalkyl having in each case 1 to 6 fluorine-, chlorine- ,and/or bromine atoms represents (C₁-C₃-halogenoalkyl)carbonyl-C₁-C₃-halogenoalkyl, (C₁-C₃-halogenoalkoxy)-carbonyl-C₁-C₃-halogenoalkyl having in each case 1 to 13 fluorine-, chlorine-, and/or bromine atoms; or represents —COR⁵, —CONR⁶R⁷, or —CH₂NR⁸R⁹,
R⁵ represents hydrogen, C₁-C₈-alkyl, C₁-C₈-alkoxy, C₁-C₄-alkoxy-C₁-C₄-alkyl, or C₃-C₈-cycloalkyl; represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms; or represents —COR¹⁰,
R⁶ and R⁷ independently of one another represent hydrogen, C₁-C₈-alkyl, C₁-C₄-alkoxy-C₁-C₄-alkyl, or C₃-C₈-cycloalkyl; or represent C₁-C₈-halogenoalkyl, halogeno-C₁-C₄-alkoxy-C₁-C₄-alkyl, or C₃-C₈-halogenocycloalkyl having in each case 1 bis 9 fluorine-, chlorine-, and/or bromine atoms; or R⁶ and R⁷ together with the nitrogen atom to which they are attached represent a saturated 5- to 8-membered heterocycle, wherein the heterocycle optionally has 1 or 2 additional non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R⁸ and R⁹ independently of one another represent hydrogen, C₁-C₈-alkyl, or C₃-C₈-cycloalkyl; or represent C₁-C₈-halogenoalkyl, C₃-C₈-halogenocycloalkyl having in each case 1 to 9 fluorine-, chlorine- and/or bromine atoms; or R⁸ and R⁹ together with the nitrogen atom to which they are attached, represent a saturated 5- to 8-membered heterocycle, wherein the heterocycle optionally has 1 or 2 additional, non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R¹⁰ represents hydrogen, C₁-C₈-alkyl, C₁-C₈-alkoxy, C₁-C₄-alkoxy-C₁-C₄-alkyl, or C₃-C₈-cycloalkyl; or represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms, and
R¹¹ represents hydrogen or C₁-C₆-alkyl.

41. an amine of the formula 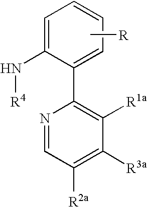
in which
R represents hydrogen, fluorine, chlorine, methyl, or trifluoromethyl;
R^{1a}, R^{2a}, and R^{3a} independently of one another represent halogen, cyano, nitro, amino, hydroxyl, formyl, carboxyl, carbamoyl, or thiocarbamoyl; represent straight-chain or branched alkyl, hydroxyalkyl, oxoalkyl, alkoxy, alkoxyalkyl, alkylthioalkyl, dialkoxyalkyl, alkylthio, alkylsulfinyl, or alkylsulfonyl having in each case 1 to 8 carbon atoms in the respective alkyl moiety; represent straight-chain or branched alkenyl or alkenyloxy having in each case 2 to 6 carbon atoms; represent straight-chain or branched halogenoalkyl, halogenoalkoxy, halogenoalkylthio, halogenoalkylsulfinyl, or halogenoalkylsulfonyl having in each case 1 to 6 carbon atoms and 1 to 13 identical or different halogen atoms; represent straight-chain or branched halogenoalkenyl or halogenoalkenyloxy having in each case 2 to 6 carbon atoms and 1 to 11 identical or different halogen atoms; represent straight-chain or branched alkylamino, dialkylamino, alkylcarbonyl, alkylcarbonyloxy, alkoxycarbonyl, alkylaminocarbonyl, dialkylaminocarbonyl, arylalkylaminocarbonyl, or dialkylaminocarbonyloxy having 1 to 6 carbon atoms in the respective hydrocarbon chain; represent alkenylcarbonyl or alkynylcarbonyl having 2 to 6 carbon atoms in the respective hydrocarbon chain; represent cycloalkyl or cycloalkyloxy having in each case 3 to 6 carbon atoms; represents the group —C(Q¹)=N-Q², wherein
Q¹ represents hydrogen, hydroxyl, C₁-C₄-alkyl, C₁-C₄-halogenoalkyl having 1 to 9 identical or different halogen atoms, or C₃-C₆-cycloalkyl, and
Q² represents hydroxyl, amino, methylamino, phenyl, or benzyl; or represents C₁-C₄-alkyl or C₁-C₄-alkoxy, each of which is optionally substituted by halogen, cyano, hydroxyl, C₁-C₄-alkoxy, C₁-C₄-alkylthio, C₁-C₄-alkylamino, di(C₁-C₄-alkyl)amino, or phenyl; or represents C₂-C₄-alkenyloxy or C₂-C₄-alkynyloxy;
represent phenyl, phenoxy, phenylthio, benzoyl, benzoylethenyl, cinnamoyl, or heterocyclyl; or represent phenylalkyl, phenylalkyloxy, phenylalkylthio, or heterocyclylalkyl having in each case 1 to 3 carbon atoms in the respective alkyl moieties, each of which is optionally mono- to tri-substituted, identically or differently, in the ring moiety by halogen or straight-chain or branched C₁-C₄-alkyl or C₁-C₄-alkoxy;
R⁴ represents hydrogen, C₁-C₈-alkyl, C₁-C₆-alkylsulfinyl, C₁-C₆-alkylsulfonyl, C₁-C₄-alkoxy-C₁-C₄-alkyl, or C₃-C₈-cycloalkyl; represents C₁-C₆-halogenoalkyl, C₁-C₄-halogenoalkylthio, C₁-C₄-halogenoalkylsulfinyl, C₁-C₄-halogenoalkylsulfonyl, halogeno-C₁-C₄-alkoxy-C₁-C₄-alkyl, or C₃-C₈-halogenocycloalkyl having in each case 1 to 9 fluorine-, chlorine-, and/or bromine atoms; represents formyl-C₁-C₃-alkyl, (C₁-C₃-alkyl)carbonyl-C₁-C₃-alkyl, (C₁-C₃-alkoxy)carbonyl-C₁-C₃-alkyl; (C₁-C₃-halogenoalkyl)carbonyl-C₁-C₃-alkyl, or (C₁-C₃-halogenoalkoxy)carbonyl-C₁-C₃-alkyl having in each case 1 to 7 fluorine-, chlorine-, and/or bromine atoms; represents (C₁-C₃-alkyl)carbonyl-C₁-C₃-halogenoalkyl or (C₁-C₃-alkoxy)carbonyl-C₁-C₃-halogenoalkyl having in each case 1 to 6 fluorine-, chlorine- ,and/or bromine atoms represents (C₁-C₃-halogenoalkyl)carbonyl-C₁-C₃-halogenoalkyl, (C₁-C₃-halogenoalkoxy)-carbonyl-C₁-C₃-halogenoalkyl having in each case 1 to 13 fluorine-, chlorine-, and/or bromine atoms; or represents —COR⁵, —CONR⁶R⁷, or —CH₂NR⁸R⁹,
R⁵ represents hydrogen, C₁-C₈-alkyl, C₁-C₈-alkoxy, C₁-C₄-alkoxy-C₁-C₄-alkyl, or C₃-C₈-cycloalkyl; represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms; or represents —COR¹⁰,
R⁶ and R⁷ independently of one another represent hydrogen, C₁-C₈-alkyl, C₁-C₄-alkoxy-C₁-C₄-alkyl, or C₃-C₈-cycloalkyl; or represent C₁-C₈-halogenoalkyl, halogeno-C₁-C₄-alkoxy-C₁-C₄-alkyl, or C₃-C₈-halogenocycloalkyl having in each case 1 bis 9 fluorine-, chlorine-, and/or bromine atoms; or R⁶ and R⁷ together with the nitrogen atom to which they are attached represent a saturated 5- to 8-membered heterocycle, wherein the heterocycle optionally has 1 or 2 additional non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R⁸ and R⁹ independently of one another represent hydrogen, C₁-C₈-alkyl, or C₃-C₈-cycloalkyl; or represent C₁-C₈-halogenoalkyl, C₃-C₈-halogenocycloalkyl having in each case 1 to 9 fluorine-, chlorine- and/or bromine atoms; or R⁸ and R⁹ together with the nitrogen atom to which they are attached, represent a saturated 5- to 8-membered heterocycle, wherein the heterocycle optionally has 1 or 2 additional, non-adjacent heteroatoms selected from the group consisting of oxygen, sulphur, and NR¹¹, and wherein the heterocycle is optionally mono- to poly-substituted, identically or differently, by halogen or C₁-C₄-alkyl,
R¹⁰ represents hydrogen, C₁-C₈-alkyl, C₁-C₈-alkoxy, C₁-C₄-alkoxy-C₁-C₄-alkyl, or C₃-C₈-cycloalkyl; or represents C₁-C₆-halogenoalkyl, C₁-C₆-halogenoalkoxy, halogeno-C₁-C₄-alkoxy-C₁-C₄-alkyl, or C₃-C₈-halogenocycloalkyl having in each case 1 to 9 fluorine-, chlorine-, and/or bromine atoms, and
R¹¹ represents hydrogen or C₁-C₆-alkyl.
